(19) United States
(12) Patent Application Publication
(10) Pub. No.: US 2024/0397815 A1
(43) Pub. Date: Nov. 28, 2024

(54) PHOTOELECTRIC CONVERSION ELEMENT, IMAGING ELEMENT, OPTICAL SENSOR, AND COMPOUND

(71) Applicant: FUJIFILM Corporation, Tokyo (JP)

(72) Inventors: Yosuke YAMAMOTO, Ashigarakami-gun (JP); Ryo FUJIWARA, Ashigarakami-gun (JP); Hiroki SUGIURA, Ashigarakami-gun (JP)

(73) Assignee: FUJIFILM Corporation, Tokyo (JP)

(21) Appl. No.: 18/794,871

(22) Filed: Aug. 5, 2024

Related U.S. Application Data
(63) Continuation of application No. PCT/JP2023/009241, filed on Mar. 10, 2023.

(30) Foreign Application Priority Data
Mar. 10, 2022 (JP) 2022-037090

(51) Int. Cl. H10K 85/60; C07D 333/22; C07D 409/14; H10K 30/30

(52) U.S. Cl. CPC H10K 85/655; C07D 333/22; C07D 409/14; H10K 85/626; H10K 85/654; H10K 30/30

(57) ABSTRACT

An object of the present invention is to provide a photoelectric conversion element which has excellent manufacturing suitability and also has excellent photoelectric conversion efficiency, an imaging element, an optical sensor, and a compound. A photoelectric conversion element according to the present invention including in the following order, a conductive film, a photoelectric conversion film, and a transparent conductive film, in which the photoelectric conversion film contains a compound represented by Formula (1).

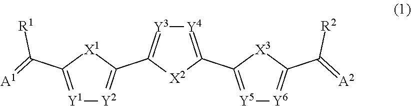

### CROSS-REFERENCE TO RELATED APPLICATIONS

This application is a Continuation of PCT International Application No. PCT/JP2023/009241 filed on Mar. 10, 2023, which claims priority under 35 U.S.C. § 119 (a) to Japanese Patent Application No. 2022-037090 filed on Mar. 10, 2022. The above applications are hereby expressly incorporated by reference, in their entirety, into the present application.

### BACKGROUND OF THE INVENTION

### 1. Field of the Invention

The present invention relates to a photoelectric conversion element, an imaging element, an optical sensor, and a compound.

### 2. Description of the Related Art

In recent years, development of an element having a photoelectric conversion film has progressed. For example, it is disclosed in WO2021/221108A that a merocyanine coloring agent is used as a material applied to a photoelectric conversion element.

### SUMMARY OF THE INVENTION

In recent years, along with the demand for improving the performance of imaging elements, optical sensors, and the like, further improvements are required for various characteristics required for photoelectric conversion elements used therein.

For example, the photoelectric conversion element is required to have excellent manufacturing suitability such that the photoelectric conversion efficiency do not deteriorate even in a case where the vapor deposition rate during the formation of the photoelectric conversion film is increased, in order to meet the requirements for manufacturing a product. In addition, a high photoelectric conversion efficiency is also required.

As a result of studying the photoelectric conversion element using the compound disclosed in WO2021/221108A and the like, the present inventor has found that it is difficult to achieve both manufacturing suitability and photoelectric conversion efficiency.

Therefore, an object of the present invention is to provide a photoelectric conversion element which has excellent manufacturing suitability and also has excellent photoelectric conversion efficiency. In addition, another object of the present invention is to provide an imaging element, an optical sensor, and a compound.

The inventors of the present invention have conducted extensive studies on the above-described problems. As a result, the inventors have found that it is possible to solve the above-described problems by applying the compound having a predetermined structure to the photoelectric conversion film, and have completed the present invention.

[1]

A photoelectric conversion element comprising in the following order, a conductive film, a photoelectric conversion film, and a transparent conductive film, in which the photoelectric conversion film contains a compound represented by Formula (1).

[2]

The photoelectric conversion element according to [1], in which R^{Y1} represents a hydrogen atom, a methyl group, an ethyl group, a linear propyl group, an aliphatic hydrocarbon group having a branched structure, an aliphatic hydrocarbon group having a cyclic structure, an aromatic ring group, an aliphatic heterocyclic group, a halogen atom, or —Si(R)₃, and the methyl group, the ethyl group, the linear propyl group, the aliphatic hydrocarbon group having a branched structure, and the aliphatic hydrocarbon group having a cyclic structure, which are represented by R^{Y1}, may have an ethereal oxygen atom.

[3]

The photoelectric conversion element according to [1] or [2], in which R^{Y2} represents a methyl group which may have a halogen atom, an ethyl group which may have a halogen atom, a linear propyl group which may have a halogen atom, an aliphatic hydrocarbon group having 3 to 6 carbon atoms, which has a branched structure and may have a halogen atom, an aliphatic hydrocarbon group having 3 to 10 carbon atoms which have a cyclic structure and may have a halogen atom, an acyl group which may have a halogen atom, —C≡C—Si(R)₃, an aromatic ring group having 10 or less carbon atoms which may have a substituent Y, an aliphatic heterocyclic group having 10 or less carbon atoms which may have a substituent Y, or —Si(R)₃, and the methyl group which may have a halogen atom, the ethyl group which may have a halogen atom, the linear propyl group which may have a halogen atom, the aliphatic hydrocarbon group having 3 to 6 carbon atoms which has a branched structure and may have a halogen atom, and the aliphatic hydrocarbon group having 3 to 10 carbon atoms which has a cyclic structure and may have a halogen atom, which are represented by R^{Y2}, may have an ethereal oxygen atom.

[4]

The photoelectric conversion element according to any one of [1] to [3], in which R^{Y2} represents a methyl group, an ethyl group, a propyl group, an isopropyl group, a tert-butyl group, a cyclopropyl group, a cyclobutyl group, a cyclopentyl group, a cyclohexyl group, an acetyl group, a trimethylsilylacetylene group, an aromatic hydrocarbon group having 10 or less carbon atoms which may have the substituent Y, or a trimethylsilyl group, and the methyl group, the ethyl group, the propyl group, the isopropyl group, the tert-butyl group, which are represented by R^{Y2}, may have an ethereal oxygen atom.

[5]

The photoelectric conversion element according to any one of [1] to [4], in which R^{Y2} represents a methyl group, an ethyl group, a propyl group, an isopropyl group, a tert-butyl group, a cyclopropyl group, a cyclobutyl group, a cyclopentyl group, a cyclohexyl group, an acetyl group, a trimethylsilylacetylene group, a phenyl group having 10 or less carbon atoms which may have the substituent Y, or a trimethylsilyl group, and the methyl group, the ethyl group, the propyl group, the isopropyl group, the tert-butyl group, which are represented by R^{Y2}, may have an ethereal oxygen atom.

[6]

The photoelectric conversion element according to any one of [1] to [5], in which X¹ to X³ represent a sulfur atom.

[7]

The photoelectric conversion element according to [1] or [2], in which the compound represented by Formula (1) includes a compound represented by any of Formula (2) to Formula (8).

[8]

The photoelectric conversion element according to any one of [1] to [7], in which A¹ and A² represent a group represented by Formula (A1) or a group represented by Formula (A2).

[9]

The photoelectric conversion element according to any one of [1] to [8], in which the photoelectric conversion film further contains an n-type organic semiconductor, and the photoelectric conversion film has a bulk hetero structure formed in a state where the compound represented by Formula (1) and the n-type organic semiconductor are mixed.

[10]

The photoelectric conversion element according to [9], in which the n-type organic semiconductor includes fullerenes selected from the group consisting of a fullerene and a derivative of the fullerene.

[11]

The photoelectric conversion element according to any one of [1] to [10], in which the photoelectric conversion film further contains a p-type organic semiconductor.

[12]

The photoelectric conversion element according to any one of [1] to [11], in which the photoelectric conversion film further contains a coloring agent.

[13]

The photoelectric conversion element according to any one of [1] to [12], further comprising one or more interlayers between the conductive film and the transparent conductive film, in addition to the photoelectric conversion film.

[14]

An imaging element comprising the photoelectric conversion element according to any one of [1] to [13].

[15]

An optical sensor comprising the photoelectric conversion element according to any one of [1] to [13].

[16]

A compound represented by Formula (1).

[17]

The compound according to [16], in which R^{Y1} represents a hydrogen atom, a methyl group, an ethyl group, a linear propyl group, an aliphatic hydrocarbon group having a branched structure, an aliphatic hydrocarbon group having a cyclic structure, an aromatic ring group, an aliphatic heterocyclic group, a halogen atom, or the —Si(R)₃, and the methyl group, the ethyl group, the linear propyl group, the aliphatic hydrocarbon group having a branched structure, and the aliphatic hydrocarbon group having a cyclic structure, which are represented by R^{Y1}, may have an ethereal oxygen atom.

[18]

The compound according to or [17], in which R^{Y2} represents a methyl group which may have a halogen atom, an ethyl group which may have a halogen atom, a linear propyl group which may have a halogen atom, an aliphatic hydrocarbon group having 3 to 6 carbon atoms which has a branched structure and may have a halogen atom, an aliphatic hydrocarbon group having 3 to 10 carbon atoms which have a cyclic structure and may have a halogen atom, an acyl group which may have a halogen atom, the —C≡C—Si(R)₃, an aromatic ring group having 10 or less carbon atoms which may have the substituent Y, an aliphatic heterocyclic group having 10 or less carbon atoms which may have the substituent Y, or the —Si(R)₃, and the methyl group which may have a halogen atom, the ethyl group which may have a halogen atom, the linear propyl group which may have a halogen atom, the aliphatic hydrocarbon group having 3 to 6 carbon atoms which has a branched structure and may have a halogen atom, and the aliphatic hydrocarbon group having 3 to 10 carbon atoms which has a cyclic structure and may have a halogen atom, which are represented by R^{Y2}, may have an ethereal oxygen atom.

[19]

The compound according to any one of to [18], in which R^{Y2} represents a methyl group, an ethyl group, a propyl group, an isopropyl group, a tert-butyl group, a cyclopropyl group, a cyclobutyl group, a cyclopentyl group, a cyclohexyl group, an acetyl group, a trimethylsilylacetylene group, an aromatic hydrocarbon group having 10 or less carbon atoms which may have the substituent Y, or a trimethylsilyl group, and the methyl group, the ethyl group, the propyl group, the isopropyl group, the tert-butyl group, which are represented by R^{Y2}, may have an ethereal oxygen atom.

[20]

The compound according to any one of to [19], in which R^{Y2} represents a methyl group, an ethyl group, a propyl group, an isopropyl group, a tert-butyl group, a cyclopropyl group, a cyclobutyl group, a cyclopentyl group, a cyclohexyl group, an acetyl group, a trimethylsilylacetylene group, a phenyl group having 10 or less carbon atoms which may have the substituent Y, or a trimethylsilyl group, and the methyl group, the ethyl group, the propyl group, the isopropyl group, the tert-butyl group, which are represented by R^{Y2}, may have an ethereal oxygen atom.

[21]

The compound according to any one of to [20], in which X¹ to X³ represent a sulfur atom.

[22]

The compound according to or [17], in which the compound represented by Formula (1) includes a compound represented by any of Formula (2) to Formula (8).

[23]

The compound according to any one of to [22], in which A¹ and A² represent a group represented by Formula (A1) or a group represented by Formula (A2).

According to the present invention, a photoelectric conversion element which has excellent manufacturing suitability and also has excellent photoelectric conversion efficiency, can be provided. In addition, according to the present invention, an imaging element, an optical sensor, and a compound can be provided.

### BRIEF DESCRIPTION OF THE DRAWINGS

FIG. 1 is a schematic cross-sectional view illustrating a configuration example of a photoelectric conversion element.

FIG. 2 is a schematic cross-sectional view illustrating a configuration example of the photoelectric conversion element.

### DESCRIPTION OF THE PREFERRED EMBODIMENTS

Hereinafter, embodiments of the photoelectric conversion element according to the present invention will be described in detail.

In the present specification, the numerical range represented by “to” means a range including numerical values denoted before and after “to” as a lower limit value and an upper limit value.

In the present specification, a hydrogen atom may be a light hydrogen atom (an ordinary hydrogen atom) or a deuterium atom (for example, a double hydrogen atom and the like).

In the present specification, in a case where there are plural substituents, linking groups, and the like (hereinafter, referred to as “substituents and the like”) represented by specific symbols, or a case where a plurality of substituents and the like are specified all together, each of the substituents and the like may be the same or may be different from each other. This also applies to a case of specifying the number of substituents and the like.

In the present specification, a “substituent” includes a group exemplified by a substituent W described later, unless otherwise specified.

### Substituent W

A substituent W in the present specification will be described below.

Examples of the substituent W include a halogen atom (for example, a fluorine atom, a chlorine atom, a bromine atom, an iodine atom, and the like), an alkyl group (including a cycloalkyl group, a bicycloalkyl group, and a tricycloalkyl group), an alkenyl group (including a cycloalkenyl group and a bicycloalkenyl group), an alkynyl group, an aryl group, a heteroaryl group (a heterocyclic group), a cyano group, a nitro group, an alkoxy group, an aryloxy group, a silyloxy group, a heterocyclic oxy group, an acyloxy group, a carbamoyloxy group, an alkoxycarbonyloxy group, an aryloxycarbonyloxy group, a secondary or tertiary amino group (including an anilino group), an alkylthio group, an arylthio group, a heterocyclic thio group, an alkyl or an arylsulfinyl group, an alkyl or an arylsulfonyl group, an acyl group, an aryloxycarbonyl group, an alkoxycarbonyl group, an aryl or a heterocyclic azo group, an imide group, a phosphino group, a phosphinyl group, a phosphinyloxy group, a phosphinylamino group, a phosphono group, a silyl group, a carboxy group, a phosphoric acid group, a sulfonic acid group, a hydroxy group, a thiol group, an acylamino group, a carbamoyl group, a ureido group, a boronic acid group, and a primary amino group. Each of the above-described groups may further have a substituent (for example, one or more groups of each of the above-described groups, and the like), as possible. For example, an alkyl group which may have a substituent is also included as a form of the substituent W.

In addition, in a case where the substituent W has a carbon atom, the number of carbon atoms of the substituent W is, for example, 1 to 20.

The number of atoms other than a hydrogen atom included in the substituent W is, for example, 1 to 30.

In addition, the specific compound described later preferably does not contain, as a substituent, a carboxy group, a salt of a carboxy group, a salt of a phosphoric acid group, a sulfonic acid group, a salt of a sulfonic acid group, a hydroxy group, a thiol group, an acylamino group, a carbamoyl group, a ureido group, or a boronic acid group (—B(OH)₂) and/or a primary amino group.

In the present specification, examples of the halogen atom include a fluorine atom, a chlorine atom, a bromine atom, and an iodine atom.

In the present specification, unless otherwise specified, the number of carbon atoms of the alkyl group is preferably 1 to 20, more preferably 1 to 10, and still more preferably 1 to 6.The alkyl group may be any of linear, branched, or cyclic.

Examples of the alkyl group include a methyl group, an ethyl group, a n-propyl group, an i-propyl group, a n-butyl group, a t-butyl group, a n-hexyl group, a cyclopentyl group, and the like.

In addition, the alkyl group may be any of a cycloalkyl group, a bicycloalkyl group, or a tricycloalkyl group, and may have a cyclic structure thereof as a partial structure.

In the alkyl group which may have a substituent, examples of a substituent which may be contained in the alkyl group include the substituent W, and an aryl group (preferably having 6 to 18 carbon atoms, and more preferably having 6 carbon atoms), a heteroaryl group (preferably having 5 to 18 carbon atoms, and more preferably having 5 and 6 carbon atoms), or a halogen atom (preferably a fluorine atom or a chlorine atom) is preferable.

In the present specification, unless otherwise specified, the above-described alkyl group is preferable as an alkyl group moiety in the alkoxy group. The alkyl group moiety in the alkylthio group is preferably the above-described alkyl group.

In the alkoxy group which may have a substituent, the substituent which may be contained in the alkoxy group includes the same examples as the substituent in the alkyl group which may have a substituent. In the alkylthio group which may have a substituent, the substituent which may be contained in the alkylthio group includes the same examples as the substituent in the alkyl group which may have a substituent.

In the present specification, the alkenyl group may be any of linear, branched, or cyclic, unless otherwise specified. The alkenyl group preferably has 2 to 20 carbon atoms. In the alkenyl group which may have a substituent, the substituent which may be contained in the alkenyl group includes the same examples as the substituent in the alkyl group which may have a substituent.

In the present specification, an alkynyl group may be any of linear, branched, or cyclic, unless otherwise specified. The alkynyl group preferably has 2 to 20 carbon atoms. In the alkynyl group which may have a substituent, the substituent which may be contained in the alkynyl group includes the same examples as the substituent in the alkyl group which may have a substituent.

In the present specification, an aromatic ring constituting the aromatic ring structure or the aromatic ring group may be any of a monocyclic ring or a polycyclic ring (for example, 2 to 6 rings or the like), unless otherwise specified. The monocyclic aromatic ring is an aromatic ring having only one aromatic ring structure as a ring structure. The polycyclic (for example, 2 to 6 rings or the like) aromatic ring is an aromatic ring formed by a plurality of (for example, 2 to 6 or the like) aromatic ring structures being fused, as a ring structure.

The number of ring member atoms of the aromatic ring is preferably 5 to 15.

The aromatic ring may be an aromatic hydrocarbon ring or an aromatic heterocyclic ring.

In a case where the aromatic ring is an aromatic heterocyclic ring, the number of heteroatoms contained as ring member atoms is, for example, 1 to 10. Examples of the heteroatoms include a nitrogen atom, a sulfur atom, an oxygen atom, a selenium atom, a tellurium atom, a phosphorus atom, a silicon atom, and a boron atom.

Examples of the aromatic hydrocarbon ring include a benzene ring, a naphthalene ring, an anthracene ring, and a phenanthrene ring.

Examples of the aromatic heterocyclic ring include a pyridine ring, a pyrimidine ring, a pyridazine ring, a pyrazine ring, a triazine ring (for example, 1,2,3-triazine ring, 1,2,4-triazine ring, 1,3,5-triazine ring, and the like), and a tetrazine ring (for example, 1,2,4,5-tetrazine ring and the like), a quinoxaline ring, a pyrrole ring, a furan ring, a thiophene ring, an imidazole ring, an oxazole ring, a thiazole ring, a benzopyrrole ring, a benzofuran ring, a benzothiophene ring, a benzimidazole ring, a benzoxazole ring, a benzothiazole ring, a naphthopyrrole ring, a naphthofuran ring, a naphthothiophene ring, a naphthimidazole ring, a naphthoxazole ring, a 3H-pyrrolidine ring, a pyrroloimidazole ring (for example, a 5H-pyrrolo [1,2-a] imidazole ring and the like), an imidazooxazole ring (for example, an imidazo [2, 1-b] oxazole ring and the like), a thienothiazole ring (for example, a thieno [2,3-d] thiazole ring and the like), a benzothiadiazole ring, a benzodithiophene ring (for example, benzo [1,2-b: 4,5-b′] dithiophene ring and the like), a thienothiophene ring (for example, thieno [3,2-b] thiophene ring and the like), a thiazolothiazole ring (for example, thiazolo [5,4-d] thiazole ring and the like), a naphthodithiophene ring (for example, a naphtho [2,3-b: 6,7-b′] dithiophene ring, a naphtho [2,1-b: 6,5-b′] dithiophene ring, a naphtho [1,2-b: 5,6-b′] dithiophene ring, a 1,8-dithiadicyclopenta [b,g] naphthalene ring, and the like), a benzothienobenzothiophene ring, a dithieno [3,2-b: 2′,3′-d] thiophene ring, and a 3,4,7,8-tetrathiadicyclopenta [a,e] pentalene ring.

In the aromatic ring which may have a substituent, examples of a type of the substituent that the aromatic ring may have include a group exemplified by the substituent W. In a case where the aromatic ring has a substituent, the number of substituents may be 1 or more (for example, 1 to 4 or the like).

In the present specification, the term “aromatic ring group” includes, for example, a group obtained by removing one or more hydrogen atoms (for example, 1 to 5 or the like) from the aromatic ring.

In the present specification, the term “aryl group” includes, for example, a group obtained by removing one hydrogen atom from a ring corresponding to an aromatic hydrocarbon ring among the above aromatic rings.

In the present specification, the term “heteroaryl group” includes, for example, a group obtained by removing one hydrogen atom from a ring corresponding to an aromatic heterocyclic ring among the above aromatic rings.

In the present specification, the term “arylene group” includes, for example, a group obtained by removing two hydrogen atoms from a ring corresponding to an aromatic hydrocarbon ring among the above aromatic rings.

In the present specification, the term “heteroarylene group” includes, for example, a group obtained by removing two hydrogen atoms from a ring corresponding to an aromatic heterocyclic ring among the above aromatic rings.

In an aromatic ring group which may have a substituent, an aryl group which may have a substituent, a heteroaryl group which may have a substituent, an arylene group which may have a substituent, and a heteroarylene group which may have a substituent, examples of a type of the substituents that these groups may have include a substituent W. In a case where these groups each of which may have a substituent have substituents, the number of substituents may be 1 or more (for example, 1 to 4 or the like).

In the present specification, in a case where a plurality of identical symbols indicating a type or the number of groups are present in Formula, which indicates a chemical structure, contents of these plurality of identical symbols indicating a type or the number of groups are independent of each other, and the contents of the identical symbols may be the same or different from each other unless otherwise specified.

In the present specification, in a case where a plurality of identical groups (for example, alkyl groups and the like) are present in one Formula, which indicates a chemical structure, specific contents between these plurality of identical groups are independent of each other, and the specific contents between the plurality of identical groups may be the same or different from each other, unless otherwise specified.

The bonding direction of the divalent group (for example, —CO—O— and the like) denoted in the present specification, is not limited unless otherwise specified. For example, in a case where Y in a compound represented by a formula “X-Y-Z” is —CO—O—, the compound may be any of “X—O—CO—Z” or “X—CO—O—Z”.

### Photoelectric Conversion Element

The photoelectric conversion element according to an embodiment of the present invention is a photoelectric conversion element including in the following order, a conductive film, a photoelectric conversion film, and a transparent conductive film, in which the photoelectric conversion film contains a compound represented by Formula (1) (hereinafter, referred to as a “specific compound”).

Examples of the feature points of the present invention include the point that the specific compound is contained, and it is presumed that, by the specific chemical structure of the specific compound, the manufacturing suitability of the photoelectric conversion film containing the specific compound is excellent, and the photoelectric conversion efficiency of the photoelectric conversion element is also excellent. In particular, it is presumed that the specific compound has a group represented by R^{Y2} in Formula (1) to exhibit the above-described effect.

Hereinafter, obtaining at least one effect of being further excellent in manufacturing suitability or being further excellent in photoelectric conversion efficiency is referred to as “the effect of the present invention is more excellent”.

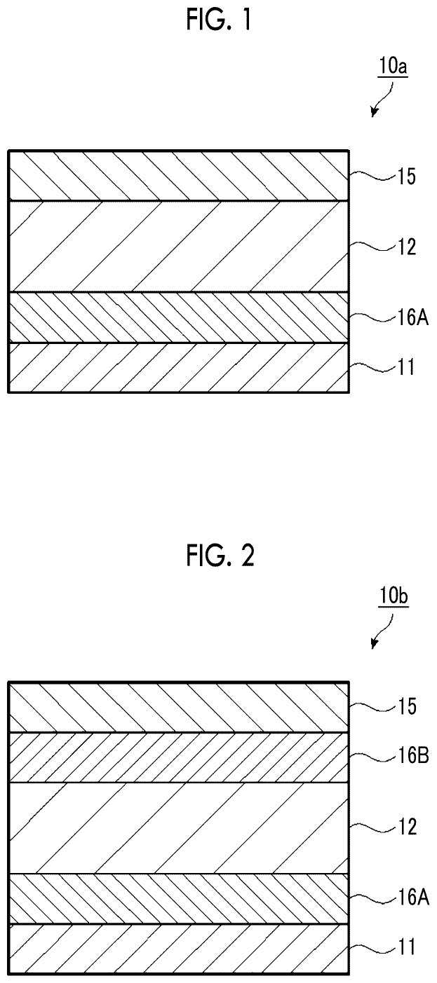

FIG. 1 is a schematic cross-sectional view of one embodiment of a photoelectric conversion element according to the embodiment of the present invention.

A photoelectric conversion element 10a illustrated in FIG. 1 has a configuration in which a conductive film (hereinafter, also referred to as a “lower electrode”) 11 functioning as a lower electrode, an electron blocking film 16A, a photoelectric conversion film 12 containing the specific compound, and a transparent conductive film (hereinafter, also referred to as an “upper electrode”) 15 functioning as an upper electrode are laminated in this order.

FIG. 2 illustrates a configuration example of another photoelectric conversion element. A photoelectric conversion element 10b illustrated in FIG. 2 has a configuration in which the electron blocking film 16A, the photoelectric conversion film 12, a positive hole blocking film 16B, and the upper electrode 15 are laminated on the lower electrode 11 in this order. The lamination order of the electron blocking film 16A, the photoelectric conversion film 12, and the positive hole blocking film 16B in FIGS. 1 and 2 may be appropriately changed according to the application and the characteristics.

In the photoelectric conversion element 10a (or 10b), it is preferable that light is incident on the photoelectric conversion film 12 through the upper electrode 15.

In a case where the photoelectric conversion element 10a (or 10b) is used, a voltage can be applied. In this case, it is preferable that the lower electrode 11 and the upper electrode 15 form a pair of electrodes, and a voltage of 1×10⁻⁵ to 1×10⁷ V/cm is applied between the pair of electrodes. From the viewpoint of the performance and power consumption, the applied voltage is more preferably 1×10⁻⁴ to 1×10⁷ V/cm, and still more preferably 1×10⁻³ to 5×10⁶ V/cm.

Regarding a voltage application method, in FIGS. 1 and 2, it is preferable that the voltage is applied such that the electron blocking film 16A side is a cathode and the photoelectric conversion film 12 side is an anode. In a case where the photoelectric conversion element 10a (or 10b) is used as an optical sensor, or also in a case where the photoelectric conversion element 10a (or 10b) is incorporated in an imaging element, the voltage can be applied by the same method.

As described in detail below, the photoelectric conversion element 10a (or 10b) can be suitably applied to applications of the imaging element.

Hereinafter, the form of each layer constituting the photoelectric conversion element according to the embodiment of the present invention will be described in detail.

### [Photoelectric Conversion Film]

The photoelectric conversion element has a photoelectric conversion film.

### Specific Compound

The photoelectric conversion film contains a specific compound.

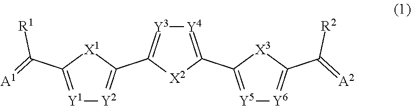

In Formula (1), Y¹ to Y⁶ each independently represent —CR^{Y1}═— or —N═. R^{Y1} represents a hydrogen atom or a substituent. At least one of Y¹, Y², Y³, Y⁴, Y⁵, or Y⁶ represents —CR^{Y2}═. R^{Y2} represents a methyl group which may have a halogen atom, an ethyl group which may have a halogen atom, a linear propyl group which may have a halogen atom, an aliphatic hydrocarbon group having a branched structure, which may have a halogen atom, an aliphatic hydrocarbon group having a cyclic structure, which may have a halogen atom, an acyl group which may have a halogen atom, —C≡C—Si(R)₃, an aromatic ring group which may have a substituent Y, an aliphatic heterocyclic group which may have a substituent Y, or —Si(R)₃.

The methyl group which may have a halogen atom, the ethyl group which may have a halogen atom, the linear propyl group which may have a halogen atom, the aliphatic hydrocarbon group which has a branched structure and may have a halogen atom, and the aliphatic hydrocarbon group which has a cyclic structure and may have a halogen atom, which are represented by R^{Y2}, may have an ethereal oxygen atom.

The substituent Y represents a methyl group, an ethyl group, a linear propyl group, an aliphatic hydrocarbon group having a branched structure, an aliphatic hydrocarbon group having a cyclic structure, an aromatic ring group, an aliphatic heterocyclic group, a halogen atom, or —Si(R)₃. The methyl group, the ethyl group, the linear propyl group, the aliphatic hydrocarbon group having a branched structure, and the aliphatic hydrocarbon group having a cyclic structure, which are represented by the substituent Y, may have an ethereal oxygen atom.

R's each independently represent a methyl group, an ethyl group, or an aromatic ring group.

X¹ to X³ each independently represent a sulfur atom, an oxygen atom, a selenium atom, or a tellurium atom.

R¹ and R² each independently represent a hydrogen atom or a substituent.

A¹ and A² each independently represent the group represented by Formula (A).

In Formula (1), Y¹ to Y⁶ each independently represent —CR^{Y1}═ or —N═. R^{Y1} represents a hydrogen atom or a substituent.

It is preferable that at least one of Y¹, Y², Y³, Y⁴, Y⁵, or Y⁶ represents —CR^{Y1}═, and it is more preferable that at least three of Y¹ to Y⁶ represent —CR^{Y1}═.

Examples of the substituent represented by R^{Y1} include a substituent exemplified by the substituent W, and a halogen atom or the substituent represented by R^{Y2} is preferable, a halogen atom is more preferable, and a fluorine atom or a chlorine atom is still more preferable.

As R^{Y1}, a hydrogen atom, a methyl group, an ethyl group, a linear propyl group, an aliphatic hydrocarbon group having a branched structure, an aliphatic hydrocarbon group having a cyclic structure, an aromatic ring group, an aliphatic heterocyclic group, a halogen atom, or —Si(R)₃ are preferable.

The methyl group, the ethyl group, the linear propyl group, the aliphatic hydrocarbon group having a branched structure, and the aliphatic hydrocarbon group having a cyclic structure, which are represented by R^{Y1}, may have an ethereal oxygen atom. As a case of having an ethereal oxygen atom, the group represented by R^{Y1} may have an ethereal oxygen atom by replacing a part of the structure of the group, or the group represented by R^{Y1} may further have an ethereal oxygen atom as another structure. Specifically, in a case where the group represented by R^{Y1} is an ethyl group, the group represented by R^{Y1} may be any of an ethoxy group or a methoxy group.

Each group represented by R^{Y1} has the same meaning as each group in the substituent Y, and suitable aspects thereof are also the same.

In a case where there is a plurality of R^{Y1}'s, R^{Y1}'s may be the same or different from each other.

In Formula (1), at least one of Y¹, Y², Y³, Y⁴, Y⁵, or Y⁶ represents —CR^{Y2}═.

At least one of Y¹, Y², Y³, Y⁴, Y⁵, or Y⁶ is —CR^{Y2}═, it is preferable that one to three of Y¹ to Y⁶ are —CR^{Y2}═, and it is more preferable that one or two of Y¹ to Y⁶ are —CR^{Y2}═.

In a case where there is a plurality of R^{Y2}'s, R^{Y2}'s may be the same or different from each other.

In Formula (1), R^{Y2} represents a methyl group which may have a halogen atom, an ethyl group which may have a halogen atom, a linear propyl group which may have a halogen atom, an aliphatic hydrocarbon group having a branched structure, which may have a halogen atom, an aliphatic hydrocarbon group having a cyclic structure, which may have a halogen atom, an acyl group which may have a halogen atom, —C≡C—Si(R)₃, an aromatic ring group which may have a substituent Y, an aliphatic heterocyclic group which may have a substituent Y, or —Si(R)₃.

The ethyl group which may have a halogen atom, the linear propyl group which may have a halogen atom, the aliphatic hydrocarbon group which has a branched structure and may have a halogen atom, and the aliphatic hydrocarbon group which has a cyclic structure and may have a halogen atom, which are represented by R^{Y2}, may have an ethereal oxygen atom.

The definition of having an ethereal oxygen atom is as described in R^{Y1}.

R^{Y2} is preferably a methyl group which may have a halogen atom, an ethyl group which may have a halogen atom, a linear propyl group which may have a halogen atom, an aliphatic hydrocarbon group having 3 to 6 carbon atoms which has a branched structure and may have a halogen atom, an aliphatic hydrocarbon group having 3 to 10 carbon atoms which have a cyclic structure and may have a halogen atom, an acyl group which may have a halogen atom, —C≡C—Si(R)₃, an aromatic ring group having 10 or less carbon atoms which may have a substituent Y, an aliphatic heterocyclic group having 10 or less carbon atoms which may have a substituent Y, or —Si(R)₃, more preferably a methyl group, an ethyl group, a propyl group, an isopropyl group, a tert-butyl group, a cyclopropyl group, a cyclobutyl group, a cyclopentyl group, a cyclohexyl group, an acetyl group, a trimethylsilylacetylene group, an aromatic hydrocarbon group having 10 or less carbon atoms which may have the substituent Y, or a trimethylsilyl group, still more preferably a methyl group, an ethyl group, a propyl group, an isopropyl group, a tert-butyl group, a cyclopropyl group, a cyclobutyl group, a cyclopentyl group, a cyclohexyl group, an acetyl group, a trimethylsilylacetylene group, a phenyl group having 10 or less carbon atoms which may have a substituent Y, or a trimethylsilyl group, particularly preferably a methyl group, an ethyl group, an isopropyl group, a tert-butyl group, a cyclopropyl group, a cyclobutyl group, a cyclopentyl group, a cyclohexyl group, an acetyl group, a trimethylsilylacetylene group, a phenyl group having 10 or less carbon atoms which may have a substituent Y, or a trimethylsilyl group, and most preferably a methyl group, an ethyl group, an isopropyl group, or a cyclopropyl group.

In addition, as another suitable aspect of R^{Y2}, a methyl group which may have a halogen atom, an ethyl group which may have a halogen atom, an aliphatic hydrocarbon group which has a cyclic structure and may have a halogen atom, or an aromatic ring group which may have a substituent Y is also preferable.

Examples of the halogen atom which can be contained in the methyl group which may have a halogen atom, the ethyl group which may have a halogen atom, the linear propyl group which may have a halogen atom, and the acyl group which may have a halogen atom include a fluorine atom, a chlorine atom, a bromine atom, and an iodine atom, and a fluorine atom or a chlorine atom is preferable.

The linear propyl group which may have a halogen atom is an n-propyl group (normal propyl group).

The aliphatic hydrocarbon group which has a branched structure and may have a halogen atom is not particularly limited as long as the aliphatic hydrocarbon group is an aliphatic hydrocarbon group having a branched structure.

The aliphatic hydrocarbon group having the branched structure preferably has 3 to 10 carbon atoms, more preferably 3 to 6 carbon atoms, and still more preferably 3 to 5 carbon atoms.

Examples of the halogen atom which can be contained in the aliphatic hydrocarbon group having a branched structure include a fluorine atom, a chlorine atom, a bromine atom, and an iodine atom, and a fluorine atom or a chlorine atom is preferable.

Examples of the aliphatic hydrocarbon group having a branched structure include an isopropyl group, a sec-butyl group, an iso-butyl group, a tert-butyl group, and a neopentyl group, and an isopropyl group or a tert-butyl group is preferable and a tert-butyl group is more preferable.

The aliphatic hydrocarbon group which has a cyclic structure and may have a halogen atom may be an aliphatic hydrocarbon group having a cyclic structure, and may further have a linear or branched aliphatic hydrocarbon group. In the present specification, the aliphatic hydrocarbon group having branched structure and cyclic structure is classified into the aliphatic hydrocarbon group which has a cyclic structure and may have a halogen atom.

The cyclic structure included in the aliphatic hydrocarbon group having the cyclic structure may be any of monocyclic or polycyclic.

The number of ring members in the cyclic structure is preferably 3 to 10 and more preferably 3 to 6.

The aliphatic hydrocarbon group having the cyclic structure preferably has 3 to 10 carbon atoms and more preferably has 3 to 6 carbon atoms.

Examples of the halogen atom which can be contained in the aliphatic hydrocarbon group having a cyclic structure include a fluorine atom, a chlorine atom, a bromine atom, and an iodine atom, and a fluorine atom or a chlorine atom is preferable.

Examples of the aliphatic hydrocarbon group having the cyclic structure include a cyclopropyl group, a cyclobutyl group, a cyclopentyl group, a cyclohexyl group, and a cycloheptyl group, and a cyclopropyl group, a cyclobutyl group, a cyclopentyl group, or a cyclohexyl group is preferable.

The acyl group which may have a halogen atom is not particularly limited as long as the acyl group is a group represented by —CO—R. R represents a substituent. Examples of the substituent include an aliphatic hydrocarbon group.

The aliphatic hydrocarbon group may be any of a linear, branched, or cyclic aliphatic hydrocarbon group.

The aliphatic hydrocarbon group preferably has 1 to 30 carbon atoms and more preferably has 1 to 10 carbon atoms.

The aliphatic hydrocarbon group is preferably a methyl group, an ethyl group, a linear propyl group, an aliphatic hydrocarbon group having a branched structure, and an aliphatic hydrocarbon group having a cyclic structure. Examples of the aliphatic hydrocarbon group having a branched structure and the aliphatic hydrocarbon group having a cyclic structure include an aliphatic hydrocarbon group having a branched structure and an aliphatic hydrocarbon group having a cyclic structure, which are represented by R^{Y2}.

As the aliphatic hydrocarbon group, a methyl group, an ethyl group, or a linear propyl group is preferable, a methyl group or an ethyl group is more preferable, and a methyl group is still more preferable.

—C≡C—Si(R)₃ is a silylacetylene group. R represents a methyl group, an ethyl group, or an aromatic ring group.
Examples of the aromatic ring group include an aromatic ring group not having the substituent Y, among the groups represented by R^{Y2}.

A plurality of R's may be the same or different from each other.

As the —C≡C—Si(R)₃, a trimethylsilylacetylene group, a triethylsilylacetylene group, a dimethylphenylsilylacetylene group, or a triphenylsilylacetylene group is preferable, and a trimethylsilylacetylene group is more preferable.

The aromatic ring group which may have the substituent Y may be any of monocyclic or polycyclic.

The aromatic ring group may be any of an aromatic hydrocarbon ring group or an aromatic heterocyclic group, and an aromatic hydrocarbon ring group is preferable.

The number of ring members in the aromatic ring group is preferably 6 to 20, more preferably 6 to 12, and still more preferably 6 to 8.

The aromatic ring group preferably has 30 or less carbon atoms, more preferably has 20 or less carbon atoms, and still more preferably has 10 or less carbon atoms. The lower limit is preferably 1 or more, more preferably 3 or more, and still more preferably 6 or more.

Examples of the heteroatom which is contained in the aromatic heterocyclic group include a sulfur atom, an oxygen atom, a nitrogen atom, a selenium atom, a tellurium atom, a phosphorus atom, a silicon atom, and a boron atom, and a sulfur atom, an oxygen atom, or a nitrogen atom is preferable.

Examples of the aromatic ring group include aromatic hydrocarbon ring groups such as a phenyl group, a naphthyl group, an anthryl group, a pyrenyl group, a phenanthryl group, a methylphenyl group, a dimethylphenyl group, a biphenyl group, and a fluorenyl group; and aromatic heterocyclic groups such as a pyridine ring group, a pyrimidine ring group, a pyridazine ring group, a pyrazine ring group, a triazine ring group, a tetrazine ring group, a quinoxaline ring group, a pyrrole ring group, a furan ring group, a thiophene ring group, an imidazole ring group, an oxazole ring group, a thiazole ring group, a benzopyrrole ring group, a benzofuran ring group, a benzothiophene ring group, a benzimidazole ring group, a benzoxazole ring group, and a benzothiazole ring group, and a phenyl group, a furan ring group, or a thiophene ring group is preferable and a phenyl group is more preferable.

As the aromatic ring group which may have the substituent Y, an aromatic hydrocarbon group having 10 or less carbon atoms which may have the substituent Y is preferable, and a phenyl group having 10 or less carbon atoms which may have the substituent Y is more preferable.

The substituent Y will be described later.

The aliphatic heterocyclic group which may have a substituent Y may be any of monocyclic or polycyclic.

The number of ring members in the aliphatic heterocyclic group is preferably 6 to 20, more preferably 6 to 12, and still more preferably 6 to 8.

The aliphatic heterocyclic group preferably has 30 or less carbon atoms, more preferably has 20 or less carbon atoms, and still more preferably has 10 or less carbon atoms.

The lower limit is preferably 1 or more, more preferably 3 or more, and still more preferably 6or more.

Examples of the heteroatom which is contained in the aliphatic heterocyclic group include a sulfur atom, an oxygen atom, a nitrogen atom, a selenium atom, a tellurium atom, a phosphorus atom, a silicon atom, and a boron atom, and a sulfur atom, an oxygen atom, or a nitrogen atom is preferable.

Examples of the aliphatic heterocyclic group include a pyrrolidine ring, an oxolane ring, a thiolane ring, a piperidine ring, a tetrahydrofuran ring, a tetrahydropyran ring, a thiane ring, a piperazine ring, a morpholine ring, a quinuclidine ring, a pyrrolidine ring, an azetidine ring, an oxetane ring, an aziridine ring, a dioxane ring, a pentamethylene sulfide ring, and γ-butyrolactone.

—Si(R)₃ is a silyl group. R represents a methyl group, an ethyl group, or an aromatic ring group.

Examples of the aromatic ring group include an aromatic ring group not having the substituent Y, among the groups represented by R^{Y2}.

A plurality of R's may be the same or different from each other.

As —Si(R)₃, a trimethylsilyl group, a triethylsilyl group, a dimethylphenylsilyl group, or a triphenylsilyl group is preferable, and a trimethylsilyl group is more preferable.

In Formula (1), the substituent Y represents a methyl group, an ethyl group, a linear propyl group, an aliphatic hydrocarbon group having a branched structure, an aliphatic hydrocarbon group having a cyclic structure, an aromatic ring group, an aliphatic heterocyclic group, a halogen atom, or —Si(R)₃. R represents a methyl group, an ethyl group, or an aromatic ring group.

Examples of the linear propyl group, the aliphatic hydrocarbon group having a branched structure, the aliphatic hydrocarbon group having a cyclic structure, the aromatic ring group, the aliphatic heterocyclic group, and —Si (R)₃, which are represented by the substituent Y, include a linear propyl group, an aliphatic hydrocarbon group which has a branched structure and has no halogen atom, an aliphatic hydrocarbon group which has a cyclic structure and has no halogen atom, an aromatic ring group not having the substituent Y, an aliphatic heterocyclic group not having the substituent Y, and —Si(R)₃, each of which is represented by R^{Y2}.

Examples of the halogen atom include a fluorine atom, a chlorine atom, a bromine atom, and an iodine atom.

As the substituent Y, a methyl group, an ethyl group, an isopropyl group, a cyclopropyl group, a fluorine atom, or a chlorine atom is preferable, a methyl group, an ethyl group, a cyclopropyl group, a fluorine atom, or a chlorine atom is more preferable, and a methyl group, a fluorine atom, or a chlorine atom is still more preferable.

In Formula (1), it is preferable that the total number of the substituents represented by R^{Y1} and the groups represented by R^{Y2} among Y¹ to Y⁶ is 3 to 6, the substituent represented by R^{Y1} represents at least a halogen atom, and the halogen atom is a halogen atom other than chlorine atom.

In addition, in Formula (1), it is also preferable that the total number of halogen atoms represented by R^{Y1} and groups represented by R^{Y2} among Y¹ to Y⁶ is 3 to 6, and the halogen atom represented by R^{Y1} is only a fluorine atom.

In Formula (1), it is also preferable that the total number of the substituents represented by R^{Y1} and the groups represented by R^{Y2} among Y¹ to Y⁶ is 2 to 6, and the substituent represented by R^{Y1} represents at least a halogen atom.

In Formula (1), it is also preferable that the total number of the substituents represented by R^{Y1} and the groups represented by R^{Y2} among Y¹ to Y⁶ is 2 to 5, and at least one of Y¹ to Y⁶ is —N═.

In Formula (1), X¹ to X³ each independently represent a sulfur atom, an oxygen atom, a selenium atom, or a tellurium atom.

X¹ to X³ are preferably a sulfur atom or an oxygen atom, and more preferably a sulfur atom.

It is also preferable that at least one of X¹, X², or X³ is a sulfur atom, and it is more preferable that at least two of X¹ to X³ are sulfur atoms.

In Formula (A), R¹ and R² each independently represent a hydrogen atom or a substituent.

Examples of the substituent include a substituent exemplified by the substituent W.

It is preferable that R¹ and R² represent a hydrogen atom.

In Formula (A), A¹ and A² each independently represent the group represented by Formula (A).

A¹ and A² are preferably a group represented by Formula (A1) or a group represented by Formula (A2).

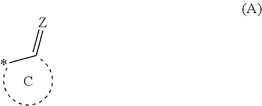

In Formula (A), * represents a bonding position. Z represents an oxygen atom, a sulfur atom, ═NR^{Z1}, or ═CR^{Z2}R^{Z3}. R^{Z1} represents a hydrogen atom or a substituent. R^{Z2} and R^{Z3} each independently represent a cyano group, —SO₂R^{Z4}, —COOR^{Z5}, or —COR^{Z6}. R^{Z4} to R^{Z6} each independently represent an aliphatic hydrocarbon group which may have a substituent, an aromatic ring group which may have a substituent, or an aliphatic heterocyclic group which may have a substituent. C represents a ring which contains 2 or more carbon atoms and may have a substituent.

In Formula (A), Z represents an oxygen atom, a sulfur atom, =NR^{Z1}, or ═CR^{Z2}R^{Z3}. R^{Z1} represents a hydrogen atom or a substituent. R^{Z2} and R23 each independently represent a cyano group, —SO₂R^{Z4}, —COOR_{Z5}, or —COR^{Z6}.

Z is preferably an oxygen atom.

Examples of the substituent represented by R^{Z1} include a substituent exemplified by the substituent W.

In Formula (A), R^{Z4} to R^{Z6} each independently represent an aliphatic hydrocarbon group which may have a substituent, an aromatic ring group which may have a substituent, or an aliphatic heterocyclic group which may have a substituent.

The aliphatic hydrocarbon group may be any of a linear, branched, or cyclic aliphatic hydrocarbon group.

As the aliphatic hydrocarbon group, an alkyl group is preferable.

The alkyl group preferably has 1 to 30 carbon atoms, more preferably has 1 to 5 carbon atoms, and particularly preferably has 1 to 3 carbon atoms.

The aromatic ring group and the aliphatic heterocyclic group may be any of monocyclic or polycyclic.

The number of ring member atoms in the aromatic ring and the aliphatic heterocyclic group is preferably 5 to 15.

Examples of the substituent which can be contained in the alkyl group, the aromatic ring group, and the aliphatic heterocyclic group include a substituent exemplified by the substituent W.

In Formula (A), C represents a ring which contains 2 or more carbon atoms and may have a substituent.

The ring preferably has 3 to 30 carbon atoms, more preferably has 3 to 20 carbon atoms, and still more preferably has 3 to 10 carbon atoms. The number of the carbon atoms is a number containing two carbon atoms specified in the formula.

The ring may be any of aromatic or non-aromatic.

The ring may be any of a monocyclic ring or a polycyclic ring, and is preferably a 5-membered ring, a 6-membered ring, or a fused ring including at least one of a 5-membered ring or a 6-membered ring. The number of rings forming the fused ring is preferably 1 to 4, and more preferably 1 to 3.

The ring may have a heteroatom. Examples of the heteroatom include a nitrogen atom, a sulfur atom, an oxygen atom, a selenium atom, a tellurium atom, a phosphorus atom, a silicon atom, and a boron atom, and a sulfur atom, a nitrogen atom, or an oxygen atom is preferable.

The number of heteroatoms in the ring is preferably 0 to 10 and more preferably 0 to 5.

In the above ring, carbon atoms constituting the ring may be substituted with another carbonyl carbon (>C═O) and/or another thiocarbonyl carbon (>C═S). The other carbonyl carbon (>C═O) and the other thiocarbonyl carbon (>C═S) mean a carbonyl carbon and a thiocarbonyl carbon each of which has a carbon atom other than the carbon atom at * part and the carbon atom bonded to Z among the carbon atoms constituting the ring, as a constituent.

Examples of the substituent which can be contained in the ring include a substituent exemplified by the substituent W, and a halogen atom, an alkyl group, an aromatic ring group, or a silyl group is preferable and a halogen atom or an alkyl group is more preferable.

The alkyl group may be linear, branched, or cyclic, and is preferably linear.

The alkyl group preferably has 1 to 10 carbon atoms and more preferably has 1 to 3 carbon atoms.

As the ring, for example, a ring which is used as an acidic nucleus (for example, an acidic nucleus of a merocyanine coloring agent) is preferable, and specific examples thereof include the following nuclei.
(a) 1,3-dicarbonyl nuclei: for example, a 1,3-indandione nucleus, 1,3-cyclohexanedione, 5,5-dimethyl-1,3-cyclohexanedione, 1,3-dioxane-4,6-dione, and the like;
(b) pyrazolinone nuclei: for example, 1-phenyl-2-pyrazolin-5-one, 3-methyl-1-phenyl-2-pyrazolin-5-one, 1-(2-benzothiazolyl)-3-methyl-2-pyrazolin-5-one, and the like;
(c) isoxazolinone nuclei: for example, 3-phenyl-2-isoxazolin-5-one, 3-methyl-2-isoxazolin-5-one, and the like;
(d) oxindole nuclei: for example, 1-alkyl-2,3-dihydro-2-oxindole and the like;
(e) 2,4,6-trioxohexahydropyrimidine nuclei: for example, barbituric acid, 2-thibarbituric acid and derivatives thereof, and the like, and examples of the derivatives include 1-alkyl compounds such as 1-methyl and 1-ethyl, 1,3-dialkyl compounds such as 1,3-dimethyl, 1,3-diethyl, and 1,3-dibutyl, 1,3-diaryl compounds such as 1,3-diphenyl, 1,3-di (p-chlorophenyl), and 1,3-di (p-ethoxycarbonylphenyl), 1-alkyl-1-aryl compounds such as 1-ethyl-3-phenyl, and 1,3-diheteroaryl compounds such as 1,3-di (2-pyridyl);
(f) 2-thio-2,4-thiazolidinedione nuclei: for example, rhodanine and derivatives thereof, and the like, and examples of the derivatives include 3-aklylrhodanine such as 3-methylrhodanine, 3-ethylrhodanine, and 3-allylrhodanine, 3-arylrhodanine such as 3-phenylrhodanine, 3-heteroarylrhodanine such as 3-(2-pyridyl) rhodanine, and the like;
(g) 2-thio-2,4-oxazolidinedione nuclei (2-thio-2,4-(3H,5H)-oxazoledione nuclei): for example, 3-ethyl-2-thio-2,4-oxazolidinedione, and the like;
(h) thianaphthenone nuclei: for example, 3 (2H)-thianaphthenone-1,1-dioxide, and the like;
(i) 2-thio-2,5-thiazolidinedione nuclei: for example, 3-ethyl-2-thio-2,5-thiazolidinedione, and the like;
(j) 2,4-thiazolidinedione nuclei: for example, 2,4-thiazolidinedione, 3-ethyl-2,4-thiazolidinedione, 3-phenyl-2,4-thiazolidinedione, and the like;
(k) thiazoliin-4-one nuclei: for example, 4-thiazolinone, 2-ethyl-4-thiazolinone, and the like;
(l) 2,4-imidazolidinedione (hydantoin) nuclei: for example, 2,4-imidazolidinedione, 3-ethyl-2,4-imidazolidinedione, and the like;
(m) 2-thio-2,4-imidazolidinedione (2-thiohydantoin) nuclei: for example, 2-thio-2,4-imidazolidinedione, 3-ethyl-2-thio-2,4-imidazolidinedione, and the like;
(n) imidazolin-5-one nuclei: for example, 2-propylmercapto-2-imidazolin-5-one, and the like;
(o) 3,5-pyrazolidinedione nuclei: for example, 1,2-diphenyl-3,5-pyrazolidinedione, 1,2-dimethyl-3,5-pyrazolidinedione, and the like;
(p) benzothiophen-3 (2H)-one nuclei: for example, benzothiophen-3 (2H)-one, oxobenzothiophen-3 (2H)-one, dioxobenzothiophen-3 (2H)-one, and the like;
(q) indanone nuclei: for example, 1-indanone, 3-phenyl-1-indanone, 3-methyl-1-indanone, 3,3-diphenyl-1-indanone, 3,3-dimethyl-1-indanone, and the like;
(r) benzofuran-3-(2H)-one nucleus: for example, benzofuran-3-(2H)-one, and the like; and(s)
2,2-dihydrophenalene-1,3-dione nucleus, and the like.

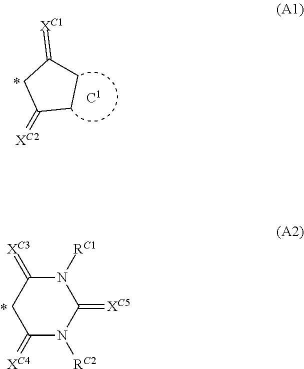

In Formula (A1), * represents a bonding position. X^{C1} and X^{C2} each independently represent an oxygen atom, a sulfur atom, ═NR^{Z1}, or ═CR^{Z2}R^{Z23}. R^{Z1} represents a hydrogen atom or a substituent. R^{Z2} and R^{Z3} each independently represent a cyano group, —SO₂R_{Z4}, —COOR^{Z5}, or —COR²⁶. R^{Z4} to R^{Z6} each independently represent an aliphatic hydrocarbon group which may have a substituent, an aromatic ring group which may have a substituent, or an aliphatic heterocyclic group which may have a substituent. C¹ represents an aromatic ring group which contains 2 or more carbon atoms and may have a substituent, or an aliphatic heterocyclic group which contains 2 or more carbon atoms and may have a substituent.

In Formula (A2), * represents a bonding position. X^{C3} to X^{C5} each independently represent an oxygen atom, a sulfur atom, ═NR^{Z1}, or —CR^{Z2}R^{Z3}. R^{Z1} represents a hydrogen atom or a substituent. R^{Z2} and R^{Z3} each independently represent a cyano group, —SO₂R^{Z4}, —COOR^{Z5}, or —COR^{Z6}. R^{Z4} to R^{Z6} each independently represent an aliphatic hydrocarbon group which may have a substituent, an aromatic ring group which may have a substituent, or an aliphatic heterocyclic group which may have a substituent. R^{C1} and R^{C2} each independently represent a hydrogen atom or a substituent.

In Formula (A1), X^{C1} and X^{C2} each independently represent an oxygen atom, a sulfur atom, ═NR^{Z1}, or ═CR^{Z2}R^{Z3}.

X^{C1} and X^{C2} are each preferably an oxygen atom.

In Formula (A1), examples of ═NR^{Z1} and ═CR^{Z2}R^{Z3} include ═NR^{Z1} and ═CR^{Z2}R^{Z3} in Formula (A).

In Formula (A1), C¹ represents an aromatic ring group which contains 2 or more carbon atoms and may have a substituent, or an aliphatic heterocyclic group which contains 2 or more carbon atoms and may have a substituent.

The aromatic ring group may be any of monocyclic or polycyclic.

The aromatic ring group preferably has 6 to 30 carbon atoms, more preferably has 6 to 12 carbon atoms, and still more preferably has 6 to 8 carbon atoms. The number of the carbon atoms is a number containing two carbon atoms specified in the formula.

Examples of the aromatic ring group include an aromatic hydrocarbon ring group and an aromatic heterocyclic group, and an aromatic hydrocarbon ring group such as a benzene ring group, a naphthalene ring group, an anthracene ring group, and a pyrene ring group is preferable, and a benzene ring group is more preferable. In addition, examples of the aromatic ring group also include an aromatic ring group which may have a substituent Y represented by R^{Y2}.

Examples of the substituent which can be contained in the aromatic ring include a substituent exemplified by the substituent W.

The aliphatic heterocyclic group may be any of monocyclic or polycyclic.

The aliphatic heterocyclic group preferably has 6 to 30 carbon atoms, more preferably has 6 to 12 carbon atoms, and still more preferably has 6 to 8 carbon atoms. The number of the carbon atoms is a number containing two carbon atoms specified in the formula.

Examples of the aliphatic heterocyclic group include an aliphatic heterocyclic group which may have a substituent Y represented by R^{Y2}.

Examples of the substituent which can be contained in the aliphatic heterocyclic group include a substituent exemplified by the substituent W.

In Formula (A2), X^{C3} to X^{C5} each independently represent an oxygen atom, a sulfur atom, ═NR^{Z1}, or ═CR^{Z2}R^{Z3}.

X^{C3} to X^{C5} are preferably an oxygen atom.

In Formula (A2), examples of ═NR^{Z1} and ═CR^{Z2}R^{Z3} include ═NR^{Z1} and ═CR^{Z2}R^{Z3} in Formula (A).

In Formula (A2), R^{C1} and R^{C2} each independently represent a hydrogen atom or a substituent.

Examples of the substituent include a substituent exemplified by the substituent W, and an alkyl group is preferable.

The alkyl group may be linear, branched, or cyclic, and is preferably linear.

The alkyl group preferably has 1 to 10 carbon atoms, more preferably has 1 to 5 carbon atoms, and still more preferably has 1 to 3 carbon atoms.

Hereinafter, A¹ and A² will be described in detail. For example, in Formula (1), in a case where A¹ and A² are each a group represented by Formula (A), a group represented by Formula (A1), or a group represented by Formula (A2), A¹ and A² each means a compound represented by Formula (A-S), a compound represented by Formula (A1-S), or a compound represented by Formula (A2-S).

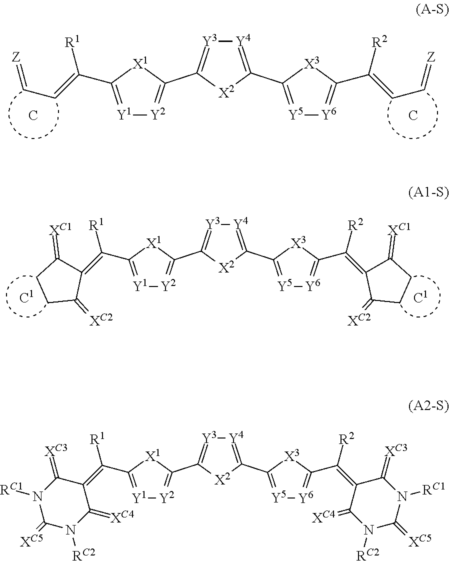

The compound represented by Formula (1) preferably includes a compound represented by any of Formula (2) to Formula (8), and more preferably includes a compound represented by any of Formula (2) to Formula (5), and Formula (8).

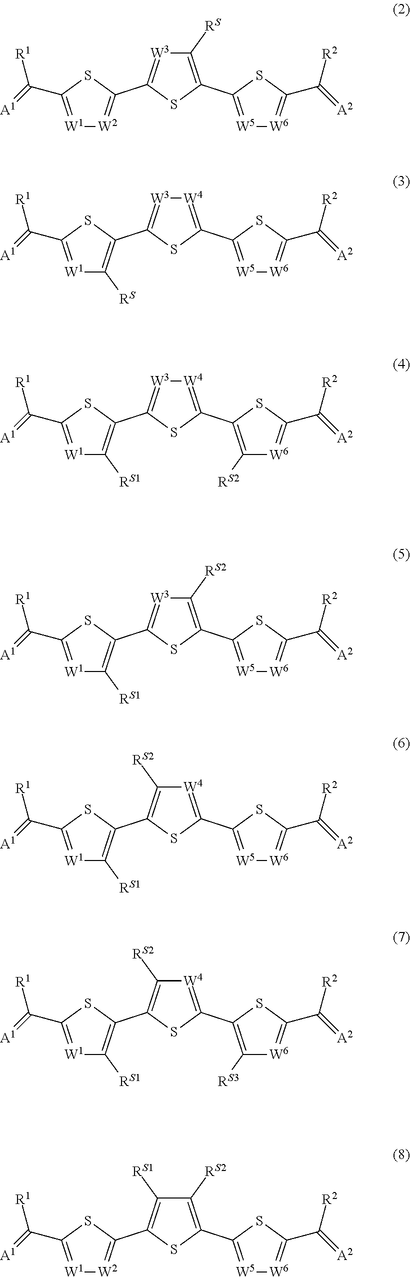

In Formula (2), W¹ to W³, W⁵, and W⁶ each independently represent —CR^{Y3}═ or a nitrogen atom. R^{Y3} represents a hydrogen atom, a fluorine atom, or a chlorine atom. R^{S} represents a methyl group which may have a halogen atom, an ethyl group which may have a halogen atom, a linear propyl group which may have a halogen atom, an aliphatic hydrocarbon group which has a branched structure and may have a halogen atom, an aliphatic hydrocarbon group which has a cyclic structure and may have a halogen atom, an acyl group which may have a halogen atom, —C≡C—Si(R)₃, an aromatic ring group which may have a substituent Y, an aliphatic heterocyclic group which may have a substituent Y, or —Si(R)₃,

The methyl group which may have a halogen atom, the ethyl group which may have a halogen atom, the linear propyl group which may have a halogen atom, the aliphatic hydrocarbon group which has a branched structure and may have a halogen atom, and the aliphatic hydrocarbon group which has a cyclic structure and may have a halogen atom, which are represented by R^{S}, may have an ethereal oxygen atom.

R¹ and R² each independently represent a hydrogen atom or a substituent.

A¹ and A² each independently represent the group represented by Formula (A).

In Formula (3), W¹ and W³ to W⁶ each independently represent —CR^{Y3}═ or a nitrogen atom. R^{Y3} represents a hydrogen atom, a fluorine atom, or a chlorine atom. R$ represents a methyl group which may have a halogen atom, an ethyl group which may have a halogen atom, a linear propyl group which may have a halogen atom, an aliphatic hydrocarbon group which has a branched structure and may have a halogen atom, an aliphatic hydrocarbon group which has a cyclic structure and may have a halogen atom, an acyl group which may have a halogen atom, —C≡C—Si(R)₃, an aromatic ring group which may have a substituent Y, an aliphatic heterocyclic group which may have a substituent Y, or —Si(R)₃,

The methyl group which may have a halogen atom, the ethyl group which may have a halogen atom, the linear propyl group which may have a halogen atom, the aliphatic hydrocarbon group which has a branched structure and may have a halogen atom, and the aliphatic hydrocarbon group which has a cyclic structure and may have a halogen atom, which are represented by R^{S}, may have an ethereal oxygen atom.

R¹ and R² each independently represent a hydrogen atom or a substituent.

A¹ and A² each independently represent the group represented by Formula (A).

In Formula (4), W¹, W³, W⁴, and W⁶ each independently represent —CR^{Y3}═ or a nitrogen atom. R^{Y3} represents a hydrogen atom, a fluorine atom, or a chlorine atom. R^{S1} and R^{S2} each independently represent a methyl group which may have a halogen atom, an ethyl group which may have a halogen atom, a linear propyl group which may have a halogen atom, an aliphatic hydrocarbon group which has a branched structure and may have a halogen atom, an aliphatic hydrocarbon group which has a cyclic structure and may have a halogen atom, an acyl group which may have a halogen atom, —C≡C—Si(R)₃, an aromatic ring group which may have a substituent Y, an aliphatic heterocyclic group which may have a substituent Y, or —Si(R)₃,

The methyl group which may have a halogen atom, the ethyl group which may have a halogen atom, the linear propyl group which may have a halogen atom, the aliphatic hydrocarbon group which has a branched structure and may have a halogen atom, and the aliphatic hydrocarbon group which has a cyclic structure and may have a halogen atom, which are represented by any of R^{S1} or R^{S2}, may have an ethereal oxygen atom.

R¹ and R² each independently represent a hydrogen atom or a substituent.

A¹ and A² each independently represent the group represented by Formula (A).

In Formula (5), W¹, W³, W⁵, and W⁶ each independently represent —CR^{Y3}═ or a nitrogen atom. R^{Y3} represents a hydrogen atom, a fluorine atom, or a chlorine atom. R^{S1} and R^{S2} each independently represent a methyl group which may have a halogen atom, an ethyl group which may have a halogen atom, a linear propyl group which may have a halogen atom, an aliphatic hydrocarbon group which has a branched structure and may have a halogen atom, an aliphatic hydrocarbon group which has a cyclic structure and may have a halogen atom, an acyl group which may have a halogen atom, —C≡C—Si(R)₃, an aromatic ring group which may have a substituent Y, an aliphatic heterocyclic group which may have a substituent Y, or —Si(R)₃,

The methyl group which may have a halogen atom, the ethyl group which may have a halogen atom, the linear propyl group which may have a halogen atom, the aliphatic hydrocarbon group which has a branched structure and may have a halogen atom, and the aliphatic hydrocarbon group which has a cyclic structure and may have a halogen atom, which are represented by any of R^{S1} or R^{S2}, may have an ethereal oxygen atom.

R¹ and R² each independently represent a hydrogen atom or a substituent.

A¹ and A² each independently represent the group represented by Formula (A).

In Formula (6), W¹ and W⁴ to W⁶ each independently represent —CR^{Y3}═ or a nitrogen atom. R^{Y3} represents a hydrogen atom, a fluorine atom, or a chlorine atom. R^{S1} and R^{S2} each independently represent a methyl group which may have a halogen atom, an ethyl group which may have a halogen atom, a linear propyl group which may have a halogen atom, an aliphatic hydrocarbon group which has a branched structure and may have a halogen atom, an aliphatic hydrocarbon group which has a cyclic structure and may have a halogen atom, an acyl group which may have a halogen atom, —C≡C—Si(R)₃, an aromatic ring group which may have a substituent Y, an aliphatic heterocyclic group which may have a substituent Y, or —Si(R)₃,

The methyl group which may have a halogen atom, the ethyl group which may have a halogen atom, the linear propyl group which may have a halogen atom, the aliphatic hydrocarbon group which has a branched structure and may have a halogen atom, and the aliphatic hydrocarbon group which has a cyclic structure and may have a halogen atom, which are represented by any of R^{S1} or R^{S2}, may have an ethereal oxygen atom.

R¹ and R² each independently represent a hydrogen atom or a substituent.

A¹ and A² each independently represent the group represented by Formula (A).

In Formula (7), W¹, W⁴, and W⁶ each independently represent —CR^{Y3}═ or a nitrogen atom. R^{Y3} represents a hydrogen atom, a fluorine atom, or a chlorine atom. R^{S1} to R^{S3} each independently represent a methyl group which may have a halogen atom, an ethyl group which may have a halogen atom, a linear propyl group which may have a halogen atom, an aliphatic hydrocarbon group which has a branched structure and may have a halogen atom, an aliphatic hydrocarbon group which has a cyclic structure and may have a halogen atom, an acyl group which may have a halogen atom, —C≡C—Si(R)₃, an aromatic ring group which may have a substituent Y, an aliphatic heterocyclic group which may have a substituent Y, or —Si(R)₃,

The methyl group which may have a halogen atom, the ethyl group which may have a halogen atom, the linear propyl group which may have a halogen atom, the aliphatic hydrocarbon group which has a branched structure and may have a halogen atom, and the aliphatic hydrocarbon group which has a cyclic structure and may have a halogen atom, which are represented by any of R^{S1} to R^{S3}, may have an ethereal oxygen atom.

R¹ and R² each independently represent a hydrogen atom or a substituent.

A¹ and A² each independently represent the group represented by Formula (A).

In Formula (8), W¹, W², W⁵, and W⁶ each independently represent —CR^{Y3}═ or a nitrogen atom. R^{Y3} represents a hydrogen atom, a fluorine atom, or a chlorine atom. R^{S1} and R^{S2} each independently represent a methyl group which may have a halogen atom, an ethyl group which may have a halogen atom, a linear propyl group which may have a halogen atom, an aliphatic hydrocarbon group which has a branched structure and may have a halogen atom, an aliphatic hydrocarbon group which has a cyclic structure and may have a halogen atom, an acyl group which may have a halogen atom, —C≡C—Si(R)₃, an aromatic ring group which may have a substituent Y, an aliphatic heterocyclic group which may have a substituent Y, or —Si(R)₃,

The methyl group which may have a halogen atom, the ethyl group which may have a halogen atom, the linear propyl group which may have a halogen atom, the aliphatic hydrocarbon group which has a branched structure and may have a halogen atom, and the aliphatic hydrocarbon group which has a cyclic structure and may have a halogen atom, which are represented by any of R^{S1} or R^{S2}, may have an ethereal oxygen atom.

R¹ and R² each independently represent a hydrogen atom or a substituent.

A¹ and A² each independently represent the group represented by Formula (A).

In Formula (2), W¹ to W³, W⁵, and W⁶ each independently represent —CR^{Y3}═ or a nitrogen atom. R^{Y3} represents a hydrogen atom, a fluorine atom, or a chlorine atom.

It is preferable that at least one of W¹, W², W³, W⁵, or W⁶ represents —CH═, and it is more preferable that at least two of W¹, W², W³, W⁵, or W⁶ represent —CH═.

A plurality of R^{Y3}'s may be the same or different from each other.

In Formula (2), examples of each group represented by R^{S}, R¹, R², A¹, and A² each include each group represented by R^{Y2}, R¹, R², A¹, and A² in Formula (1).

In Formula (3), W¹ and W³ to W⁶ each independently represent —CR^{Y3}═ or a nitrogen atom. R^{Y3} represents a hydrogen atom, a fluorine atom, or a chlorine atom.

It is preferable that at least one of W¹, W³, W⁴, W⁵, or W⁶ represents —CH═, and it is more preferable that at least two of W¹, W³, W⁴, W⁵, W⁶ represent —CH═.

A plurality of R^{Y3}'s may be the same or different from each other.

In Formula (3), examples of each group represented by R^{S}, R¹, R², A¹, and A² each include each group represented by R^{Y2}, R¹, R², A¹, and A² in Formula (1).

In Formula (4), W¹, W³, W⁴, and W⁶ each independently represent —CR^{Y3}═ or a nitrogen atom. R^{Y3} represents a hydrogen atom, a fluorine atom, or a chlorine atom.

It is preferable that at least one of W¹, W³, W⁴, or W⁶ represents —CH═, and it is more preferable that at least two of W¹, W³, W⁴, or W⁶ represent —CH═.

A plurality of R^{Y3}'s may be the same or different from each other.

In Formula (4), examples of each group represented by any of R^{S1} or R^{S2}, R¹, R², A¹, and A² each include each group represented by R^{Y2}, R¹, R², A¹, and A² in Formula (1).

In Formula (5), W¹, W³, W⁵, and W⁶ each independently represent —vCR^{Y3}═ or a nitrogen atom. R^{Y3} represents a hydrogen atom, a fluorine atom, or a chlorine atom.

It is preferable that at least one of W¹, W³, W⁵, or W⁶ represents —CH═, and it is more preferable that at least two of W¹, W³, W⁵, or W⁶ represent —CH═.

A plurality of R^{Y3}'s may be the same or different from each other.

In Formula (5), examples of each group represented by any of R^{S1} or R^{S2}, R¹, R², A¹, and A² each include each group represented by R^{Y2}, R¹, R², A¹, and A² in Formula (1).

In Formula (6), W¹ and W⁴ to W⁶ each independently represent —CR^{Y3}═ or a nitrogen atom. R^{Y3} represents a hydrogen atom, a fluorine atom, or a chlorine atom.

It is preferable that at least one of W¹, W⁴, W⁵, or W⁶ represents —CH═, and it is more preferable that at least two of W¹, W⁴, W⁵, or W⁶ represent —CH═.

A plurality of R^{Y3}'s may be the same or different from each other.

In Formula (6), examples of each group represented by any of R^{S1} or R^{S2}, R¹, R², A¹, and A² each include each group represented by R^{Y2}, R¹, R², A¹, and A² in Formula (1).

In Formula (7), W¹, W⁴, and W⁶ each independently represent —CR^{Y3}═ or a nitrogen atom.
R^{Y3} represents a hydrogen atom, a fluorine atom, or a chlorine atom.

It is preferable that at least one of W¹, W⁴, or W⁶ represents —CH═, and it is more preferable that at least two of W¹, W⁴, or W⁶ represent —CH═.

A plurality of R^{Y3}'s may be the same or different from each other.

In Formula (7), examples of each group represented by any of R^{S1} to R^{S3}, R¹, R², A¹, and A² each include each group represented by R^{Y2}, R¹, R², A¹, and A² in Formula (1).

In Formula (8), W¹, W², W³, and W⁶ each independently represent —CR^{Y3}═ or a nitrogen atom. R^{Y3} represents a hydrogen atom, a fluorine atom, or a chlorine atom.

It is preferable that at least one of W¹, W², W⁵, or W⁶ represents —CH═, and it is more preferable that at least two of W¹, W², W⁵, or W⁶ represent —CH═.

A plurality of R^{Y3}'s may be the same or different from each other.

In Formula (8), examples of each group represented by any of R^{S1} or R^{S2}, R¹, R², A¹, and A² each include each group represented by R^{Y2}, R¹, R², A¹, and A² in Formula (1).

Examples of the specific compound include the following compounds.

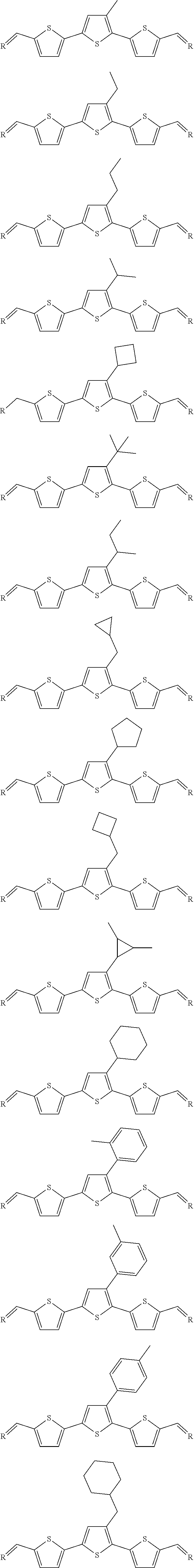

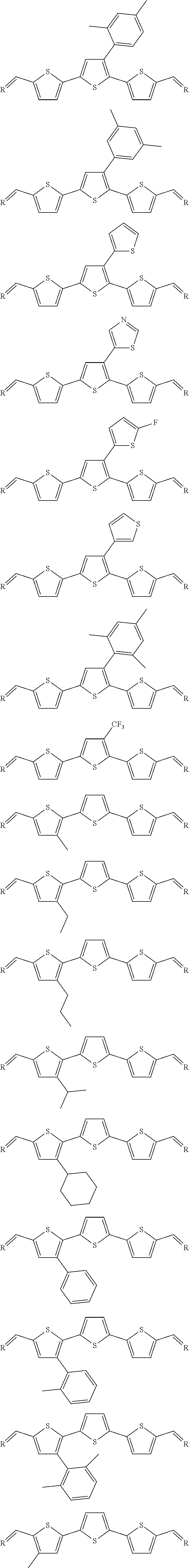

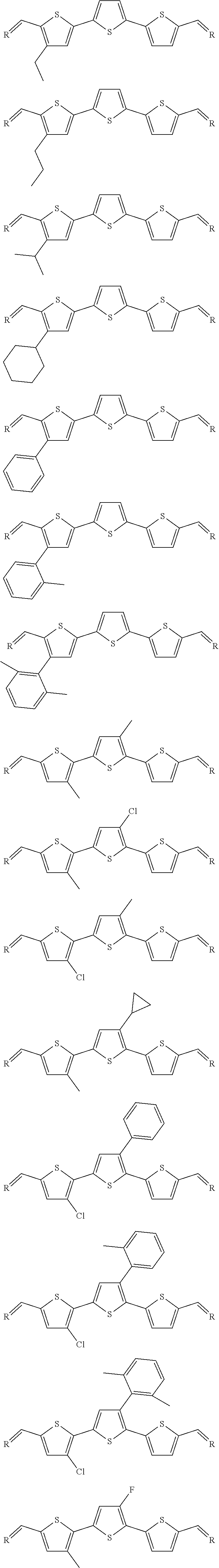

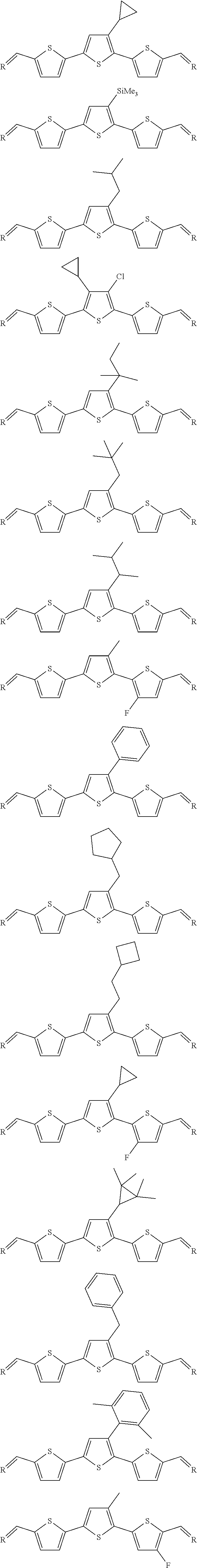

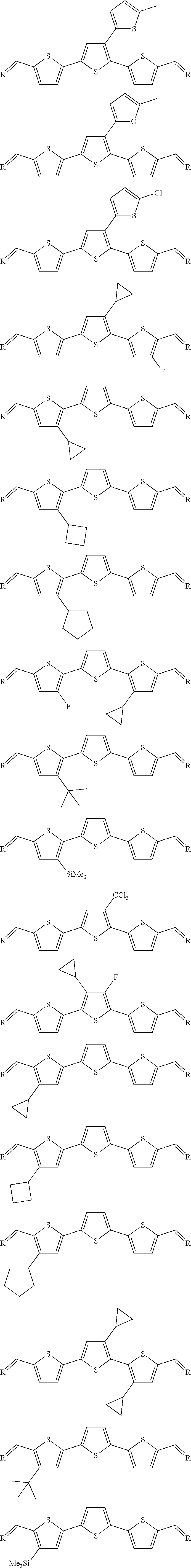

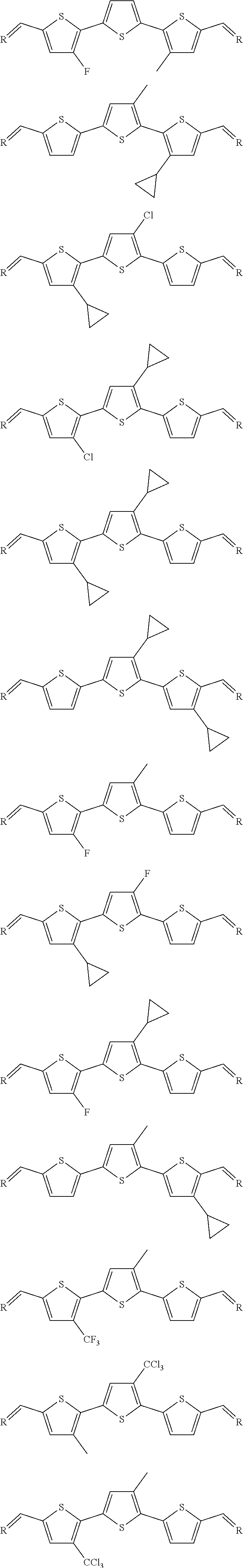

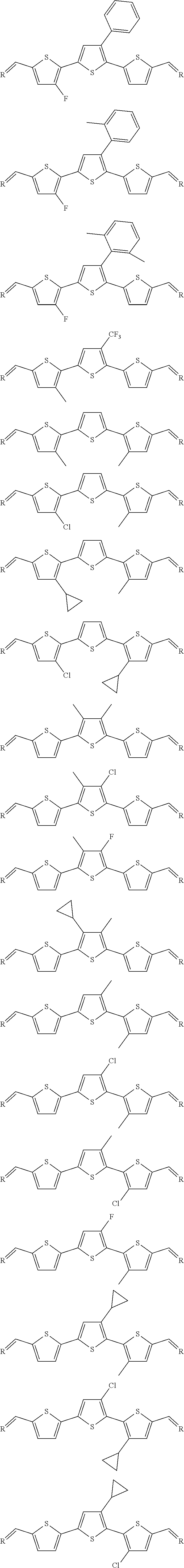

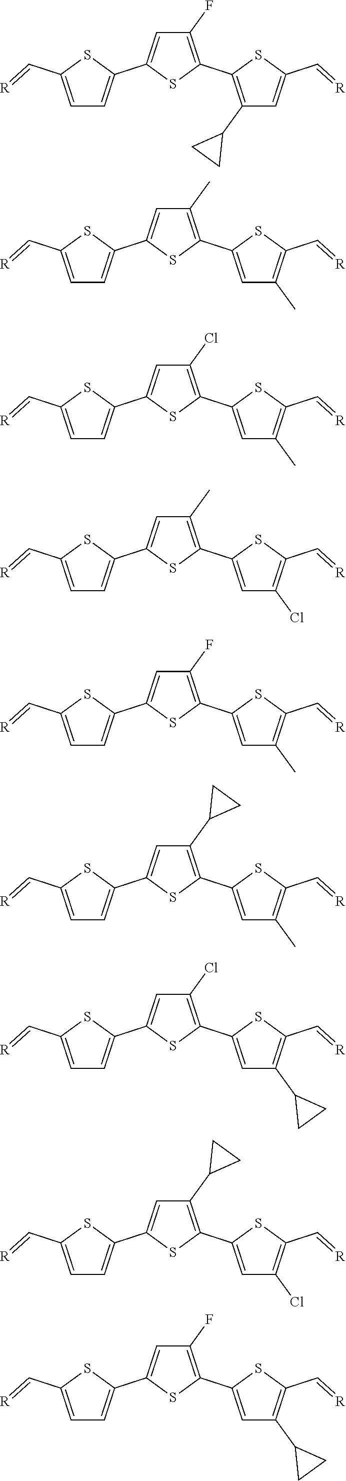

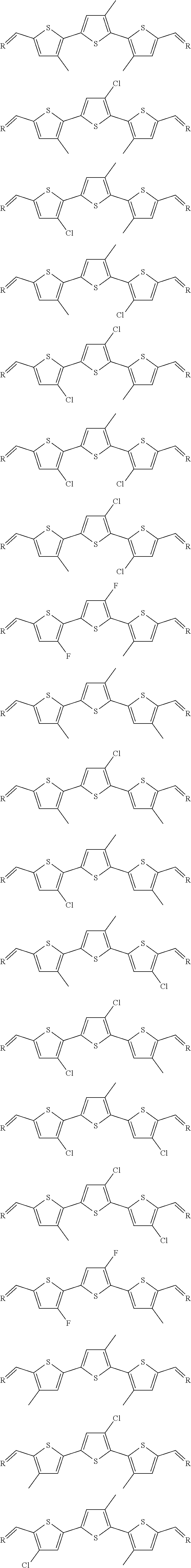

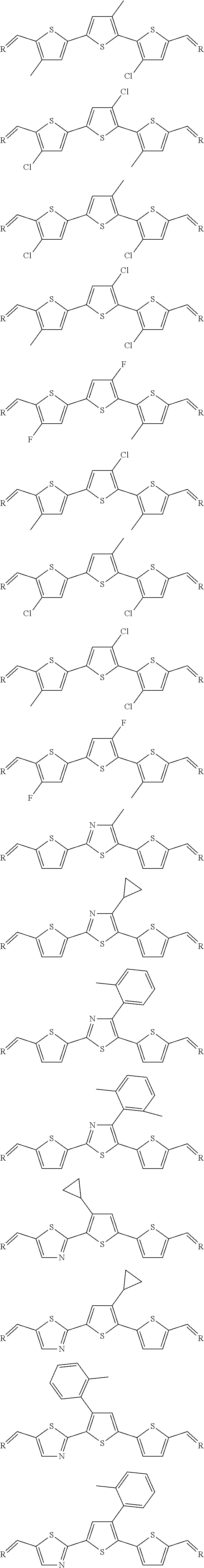

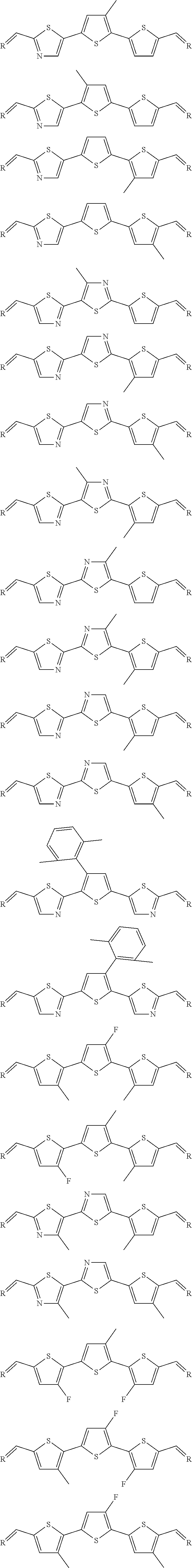

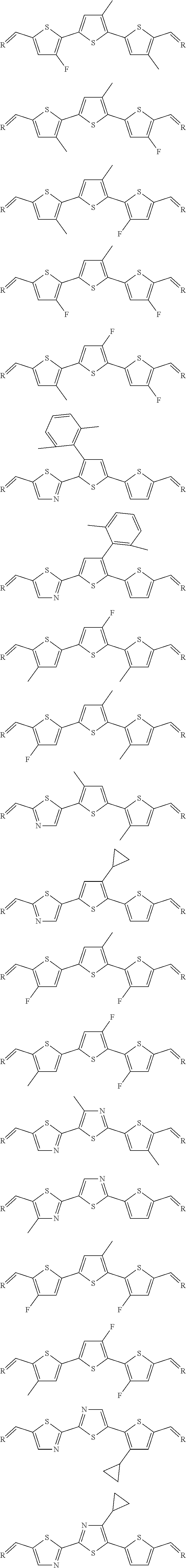

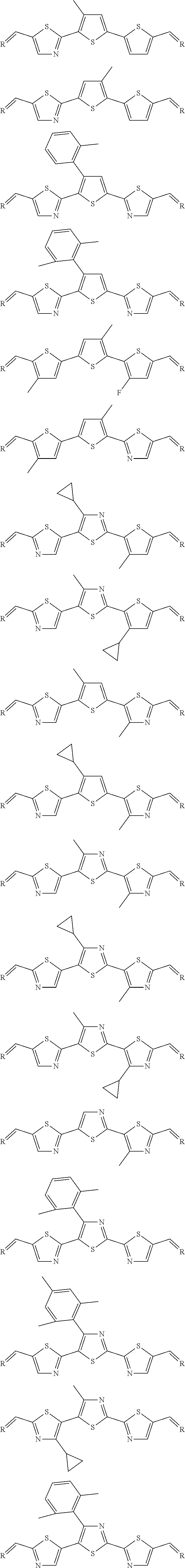

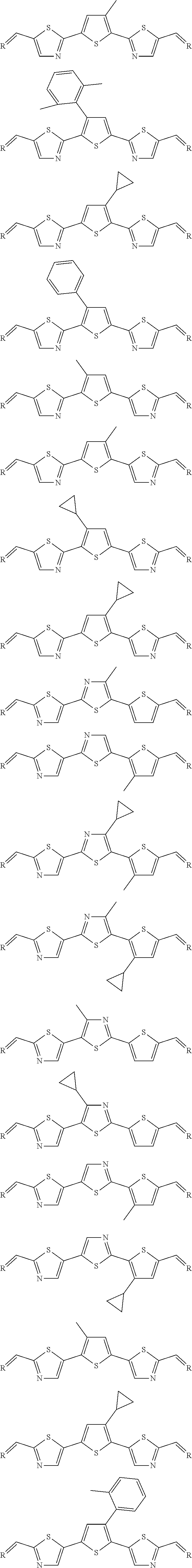

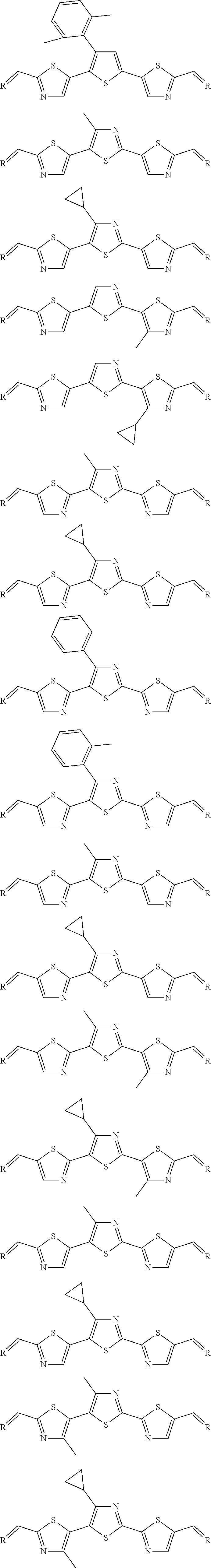

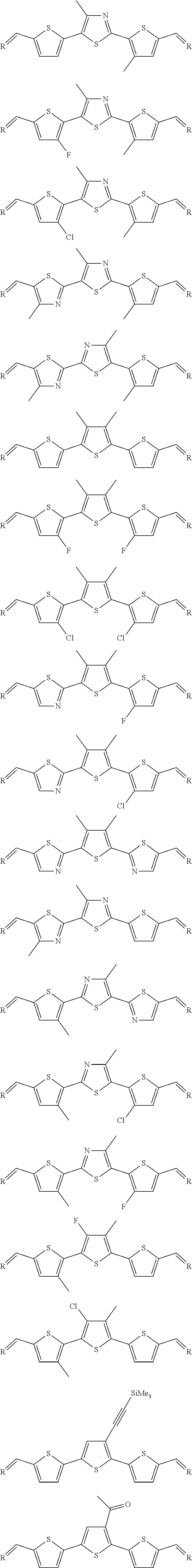

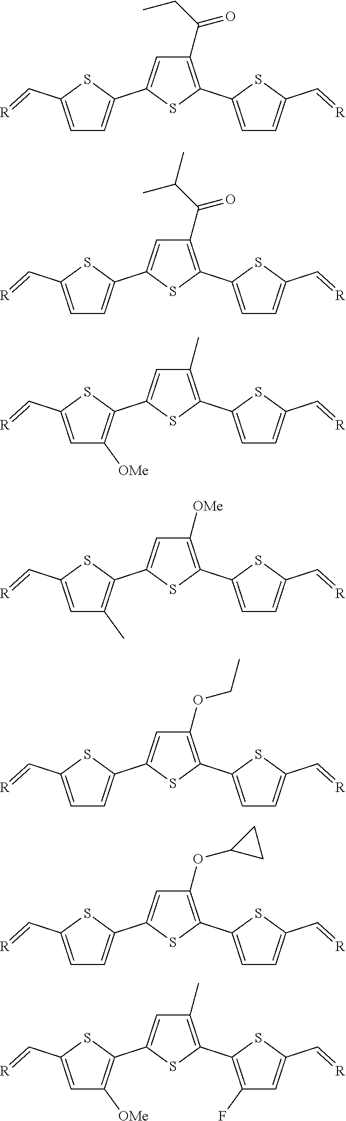

R in the Exemplary compound represents any of the following groups. * represents a bonding position.

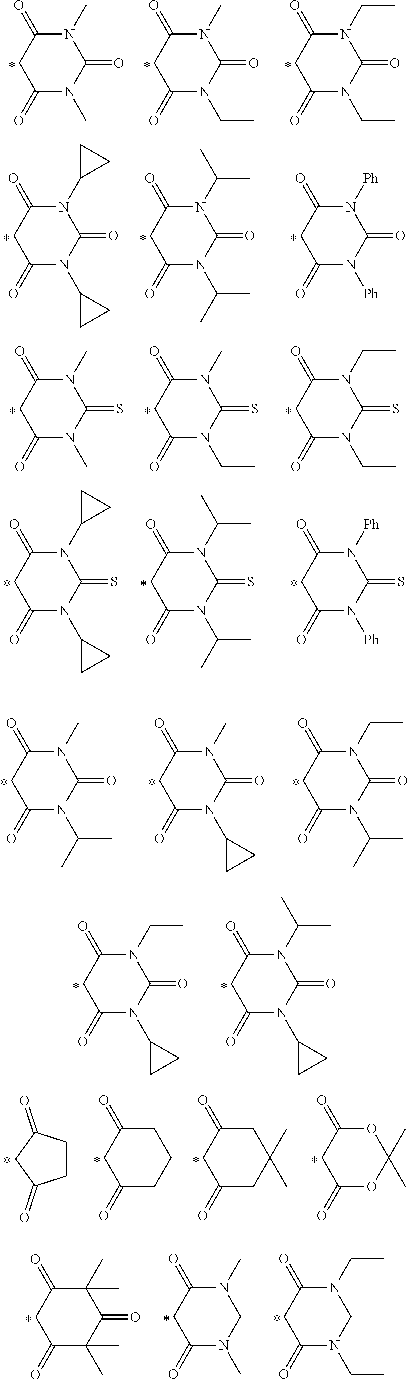

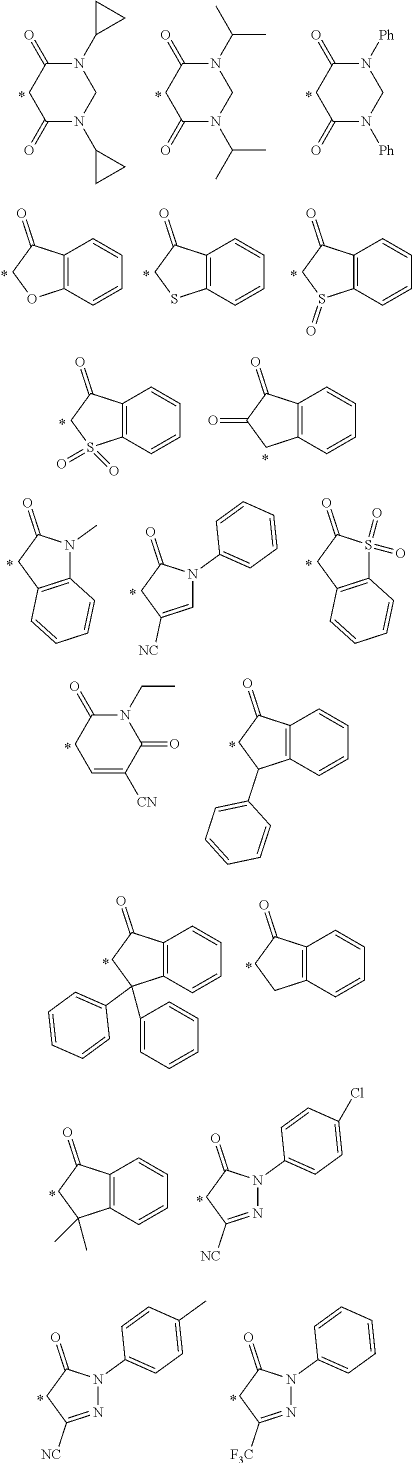

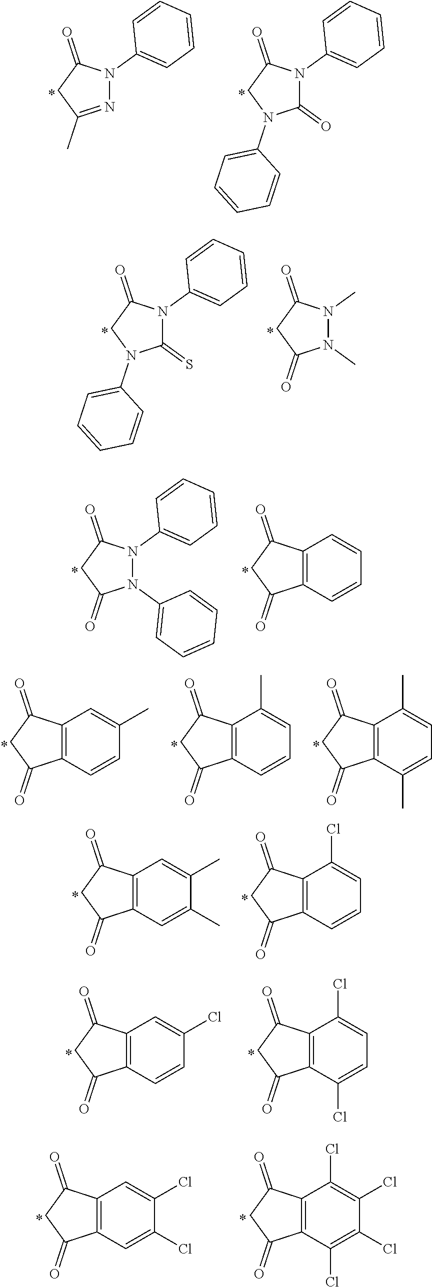

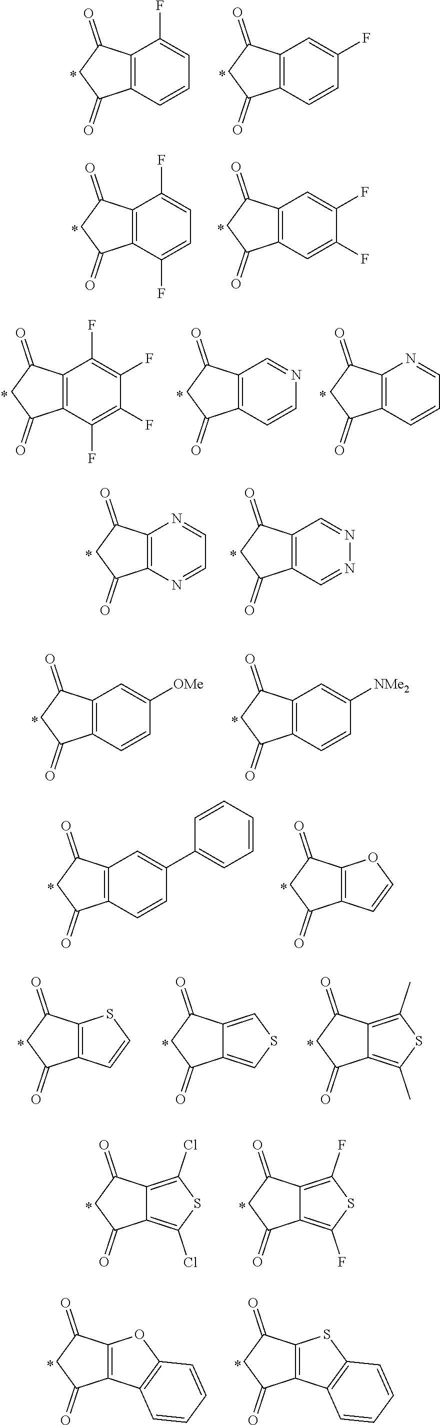

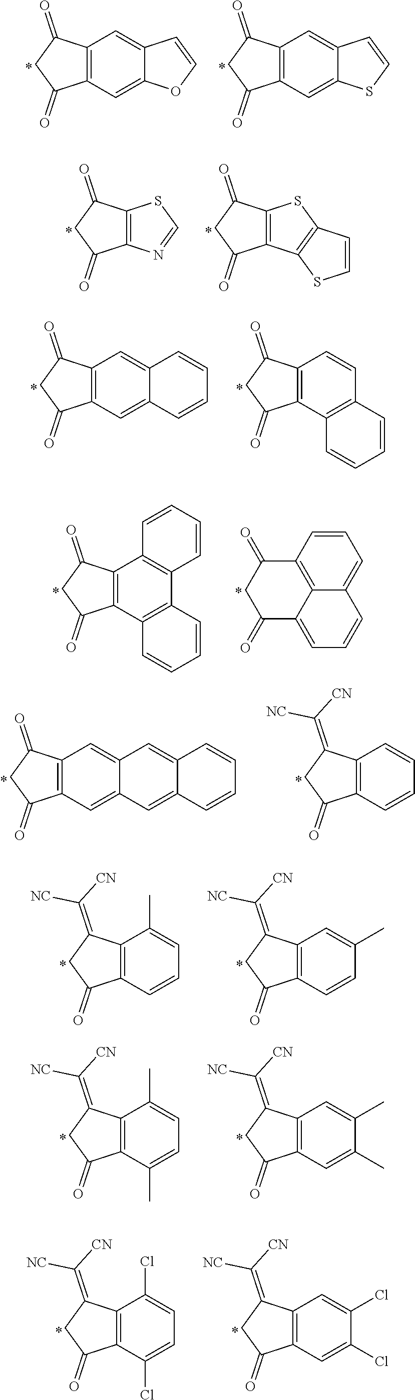

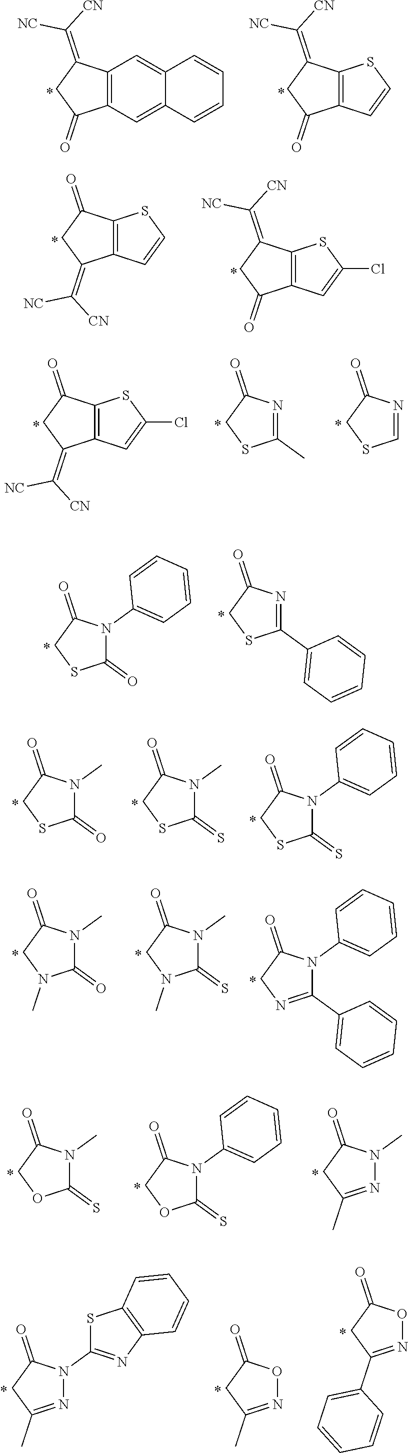

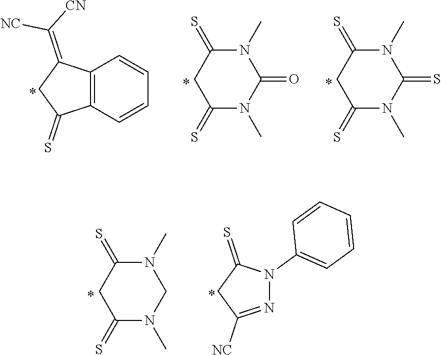

A molecular weight of the specific compound is preferably 400 to 1,200, more preferably 400 to 1,000, and still more preferably 400 to 800.

In a case where the molecular weight is in the above-described range, it is presumed that the sublimation temperature of the specific compound becomes low, and the photoelectric conversion efficiency is excellent also in a case where a photoelectric conversion film is formed at a high speed.

The specific compound is particularly useful as a material of the photoelectric conversion film used for the imaging element, the optical sensor, or a photoelectric cell. The specific compound often functions as a coloring agent in the photoelectric conversion film. The specific compound can also be used as a coloring material, a liquid crystal material, an organic semiconductor material, a charge transport material, a pharmaceutical material, and a fluorescent diagnostic material.

In the specific compound, an ionization potential in a single film is preferably −6.0 to −5.0 eV from the viewpoints of stability in a case of using the compound as the p-type organic semiconductor and matching of energy levels between the compound and the n-type organic semiconductor.

The maximal absorption wavelength of the specific compound is preferably in a wavelength range of 400 to 600 nm, and more preferably in a wavelength range of 450 to 580 nm.

The maximal absorption wavelength is a value measured in a solution state (solvent: chloroform) by an absorption spectrum of the specific compound being adjusted to a concentration having an absorbance of about 0.5 to 1.0. Provided that in a case where the specific compound is not soluble in chloroform, a value measured by using the specific compound in which the specific compound is vapor-deposited and formed into a film state is defined as a maximal absorption wavelength of the specific compound.

The specific compound may be purified as necessary.

Examples of a purification method of the specific compound include sublimation purification, purification using silica gel column chromatography, purification using gel permeation chromatography, reslurry washing, repurification by reprecipitation, purification using an adsorbent such as activated carbon, and recrystallization purification.

The specific compound may be used alone or in combination of two or more types thereof.

A content of the specific compound in the photoelectric conversion film (=film thickness of specific compound in terms of single layer/film thickness of photoelectric conversion film×100) is preferably 15% to 75% by volume, more preferably 20% to 60% by volume, and still more preferably 25% to 50% by volume.

<n-Type Organic Semiconductor>

The photoelectric conversion film preferably contains the n-type organic semiconductor in addition to the specific compound.

The n-type organic semiconductor is a compound different from the specific compound.

The n-type organic semiconductor is an acceptor-property organic semiconductor material (a compound), and refers to an organic compound having a property of easily accepting an electron. That is, the n-type organic semiconductor refers to an organic compound having a large electron affinity of two organic compounds used in contact with each other. That is, any organic compound having an electron accepting property can be used as the acceptor type organic semiconductor.

Examples of the n-type organic semiconductor include fullerenes selected from the group consisting of a fullerene and derivatives thereof, fused aromatic carbocyclic compounds (for example, a naphthalene derivative, an anthracene derivative, a phenanthrene derivative, a tetracene derivative, a pyrene derivative, a perylene derivative, and a fluoranthene derivative); a heterocyclic compound having a 5- to 7-membered ring having at least one selected from the group consisting of a nitrogen atom, an oxygen atom, and a sulfur atom (for example, pyridine, pyrazine, pyrimidine, pyridazine, triazine, quinoline, quinoxaline, quinazoline, phthalazine, cinnoline, isoquinoline, pteridine, acridine, phenazine, phenanthroline, tetrazole, pyrazole, imidazole, and thiazole); polyarylene compounds; fluorene compounds; cyclopentadiene compounds; silyl compounds; 1,4,5,8-naphthalenetetracarboxylic acid anhydride; 1,4,5,8-naphthalenetetracarboxylic acid anhydride imide derivative and oxadiazole derivatives; anthraquinodimethane derivatives; diphenylquinone derivatives; bathocuproine, bathophenanthroline, and derivatives thereof; triazole compounds; a distyrylarylene derivative; a metal complex having a nitrogen-containing heterocyclic compound as a ligand; a silole compound; and compounds disclosed in paragraphs [0056] to [0057] of JP2006-100767A.

The n-type organic semiconductor (compound) is preferably fullerenes selected from the group consisting of a fullerene and derivatives thereof.

Examples of the fullerenes include a fullerene C₆₀, a fullerene C₇₀, a fullerene C₇₆, a fullerene C₇₈, a fullerene C₈₀, a fullerene C₈₂, a fullerene C₈₄, a fullerene C₉₀, a fullerene C₉₆, a fullerene C₂₄₀, a fullerene C₅₄₀, and a mixed fullerene.

Examples of the fullerene derivatives include compounds in which a substituent is added to the above fullerenes. The substituent is preferably an alkyl group, an aryl group, or a heterocyclic group. As the fullerene derivative, the compounds described in JP2007-123707A are preferable.

The n-type organic semiconductor may be an organic coloring agent.

Examples of the organic coloring agent include a cyanine coloring agent, a styryl coloring agent, a hemicyanine coloring agent, a merocyanine coloring agent (including zeromethine merocyanine (simple merocyanine)), a rhodacyanine coloring agent, an allopolar coloring agent, an oxonol coloring agent, a hemioxonol coloring agent, a squarylium coloring agent, a croconium coloring agent, an azamethine coloring agent, a coumarin coloring agent, an arylidene coloring agent, an anthraquinone coloring agent, a triphenylmethane coloring agent, an azo coloring agent, an azomethine coloring agent, a metallocene coloring agent, a fluorenone coloring agent, a flugide coloring agent, a perylene coloring agent, a phenazine coloring agent, a phenothiazine coloring agent, a quinone coloring agent, a diphenylmethane coloring agent, a polyene coloring agent, an acridine coloring agent, an acridinone coloring agent, a diphenylamine coloring agent, a quinophthalone coloring agent, a phenoxazine coloring agent, a phthaloperylene coloring agent, a dioxane coloring agent, a porphyrin coloring agent, a chlorophyll coloring agent, a phthalocyanine coloring agent, a subphthalocyanine coloring agent, and a metal complex coloring agent.

The molecular weight of the n-type organic semiconductor is preferably 200 to 1,200, and more preferably 200 to 900.

The maximal absorption wavelength of the n-type organic semiconductor is preferably in a wavelength of 400 nm or less or in a wavelength range of 500 to 600 nm.

It is preferable that the photoelectric conversion film has a bulk hetero structure formed in a state in which the specific compound and the n-type organic semiconductor are mixed. The bulk hetero structure refers to a layer in which the specific compound and the n-type organic semiconductor are mixed and dispersed in the photoelectric conversion film. The photoelectric conversion film having the bulk hetero structure can be formed by either a wet method or a dry method. The bulk hetero structure is described in detail in, for example, paragraphs [0013] and [0014] of JP2005-303266A.

The difference in electron affinity between the specific compound and the n-type organic semiconductor is preferably 0.1 eV or more.

The n-type organic semiconductor may be used alone, or two or more types thereof may be used in combination.

In a case where the photoelectric conversion film contains the n-type organic semiconductor, a content of the n-type organic semiconductor in the photoelectric conversion film (film thickness of n-type organic semiconductor in terms of single layer/film thickness of photoelectric conversion film×100) is preferably 15% to 75% by volume, more preferably 20% to 60% by volume, and still more preferably 20% to 50% by volume.

In a case where the n-type organic semiconductor material includes fullerenes, a content of the fullerenes to a total content of the n-type organic semiconductor material (film thickness of fullerenes in terms of single layer/total film thickness of n-type organic semiconductor materials in terms of single layer×100) is preferably 50% to 100% by volume, and more preferably 80% to 100% by volume. The fullerenes may be used alone, or two or more types thereof may be used in combination.

From the viewpoint of response speed of the photoelectric conversion element, the content of the specific compound to the total content of the specific compound and the n-type organic semiconductor (film thickness in terms of single layer of specific compound/(film thickness in terms of single layer of specific compound+film thickness in terms of single layer of n-type organic semiconductor)×100) is preferably 20% to 80% by volume, and more preferably 40% to 80% by volume.

In a case where the photoelectric conversion film contains an n-type organic semiconductor and a p-type organic semiconductor, the content of the specific compound (=film thickness in terms of single layer of specific compound/(film thickness in terms of single layer of specific compound+film thickness in terms of single layer of n-type organic semiconductor+film thickness in terms of single layer of p-type organic semiconductor)×100) is preferably 15% to 75% by volume, and more preferably 30% to 75% by volume.

It is preferable that the photoelectric conversion film is substantially formed of the specific compound, the n-type organic semiconductor, and the p-type organic semiconductor included as desired. The term “substantially” indicates that the total content of the specific compound, the n-type organic semiconductor, and the p-type organic semiconductor is 90% to 100% by volume, preferably 95% to 100% by volume, and more preferably 99% to 100% by volume, with respect to the total mass of the photoelectric conversion film.

<p-Type Organic Semiconductor>

The photoelectric conversion film preferably contains the p-type organic semiconductor in addition to the specific compound.

The p-type organic semiconductor is a compound different from the specific compound.

The p-type organic semiconductor is a donor organic semiconductor material (a compound), and refers to an organic compound having a property of easily donating an electron. That is, the p-type organic semiconductor means an organic compound having a smaller ionization potential in a case where two organic compounds are used in contact with each other.

The p-type organic semiconductor may be used alone, or two or more types thereof may be used in combination.

Examples of the p-type organic semiconductor include triarylamine compounds (for example, N, N′-bis (3-methylphenyl)-(1,1′-biphenyl)-4,4′-diamine (TPD), 4,4′-bis [N-(naphthyl)-N-Phenyl-amino]biphenyl (α-NPD), compounds disclosed in paragraphs to [0128] to [0148] of JP2011-228614A, compounds disclosed in paragraphs [0052] to [0063] of JP2011-176259A, compounds disclosed in paragraphs [0119] to [0158] of JP2011-225544A, compounds disclosed in paragraphs [0044] to [0051] of JP2015-153910A, and compounds disclosed in paragraphs [0086] to [0090] of JP2012-094660A), pyrazoline compounds, styrylamine compounds, hydrazone compounds, polysilane compounds, thiophene compounds (for example, a thienothiophene derivative, a dibenzothiophene derivative, a benzodithiophene derivative, a dithienothiophene derivative, a [1]benzothieno[3,2-b]thiophene (BTBT) derivative, a thieno [3,2-f: 4,5-f′] bis [1] benzothiophene (TBBT) derivative, compounds disclosed in paragraphs [0031] to [0036] of JP2018-014474A, compounds disclosed in paragraphs [0043] to [0045] of WO2016-194630A, compounds disclosed in paragraphs [0025] to [0037], and [0099] to [0109] of WO2017-159684A, compounds disclosed in paragraphs [0029] to [0034] of JP2017-076766A, compounds disclosed in paragraphs [0015] to [0025] of WO2018-207722A, compounds disclosed in paragraphs [0045] to [0053] of JP2019-054228A, compounds disclosed in paragraphs [0045] to [0055] of WO2019-058995A, compounds disclosed in paragraphs [0063] to [0089] of WO2019-081416A, compounds disclosed in paragraphs [0033] to [0036] of JP2019-80052A, compounds disclosed in paragraphs [0044] to [0054] of WO2019-054125A, compounds disclosed in paragraphs [0041] to [0046] of WO2019-093188A, compounds in paragraphs [0034] to [0037] of JP2019-050398A, compounds in paragraphs [0033] to [0036] of JP2018-206878A, compounds in paragraph [0038] of JP2018-190755A, compounds in paragraphs [0019] to [0021] of JP2018-026559A, compounds in paragraphs [0031] to [0056] of JP2018-170487A, compounds in paragraphs [0036] to [0041] of JP2018-078270A, compounds in paragraphs [0055] to [0082] of JP2018-166200A, compounds in paragraphs [0041] to [0050] of JP2018-113425A, compounds in paragraphs [0044] to [0048] of JP2018-085430A, compounds in paragraphs [0041] to [0045] of JP2018-056546A, compounds in paragraphs [0042] to [0049] of JP2018-046267A, compounds in paragraphs [0031] to [0036] of JP2018-014474A, compounds disclosed in paragraphs [0036] to [0046] of WO2018-016465A, compounds disclosed in paragraphs [0045] to [0048] of JP2020-010024A, and the like), a cyanine compound, an oxonol compound, a polyamine compound, an indole compound, a pyrrole compound, a pyrazole compound, a polyarylene compound, a fused aromatic carbocyclic compound (for example, a naphthalene derivative, an anthracene derivative, a phenanthrene derivative, a tetracene derivative, a pentacene derivative, a pyrene derivative, a perylene derivative, a fluoranthene derivative, and the like), a porphyrin compound, a phthalocyanine compound, a triazole compound, an oxadiazole compound, an imidazole compound, a polyarylalkane compound, a pyrazolone compound, an amino-substituted chalcone compound, an oxazole compound, a fluorenone compound, a silazane compound, and a metal complex having nitrogen-containing heterocyclic compounds as ligands.

Examples of the p-type organic semiconductor also include compounds having an ionization potential smaller than that of the n-type organic semiconductor, and in a case where this condition is satisfied, the organic coloring agents exemplified as the n-type organic semiconductor can be used.

The compounds that can be used as the p-type organic semiconductor compound are exemplified below.

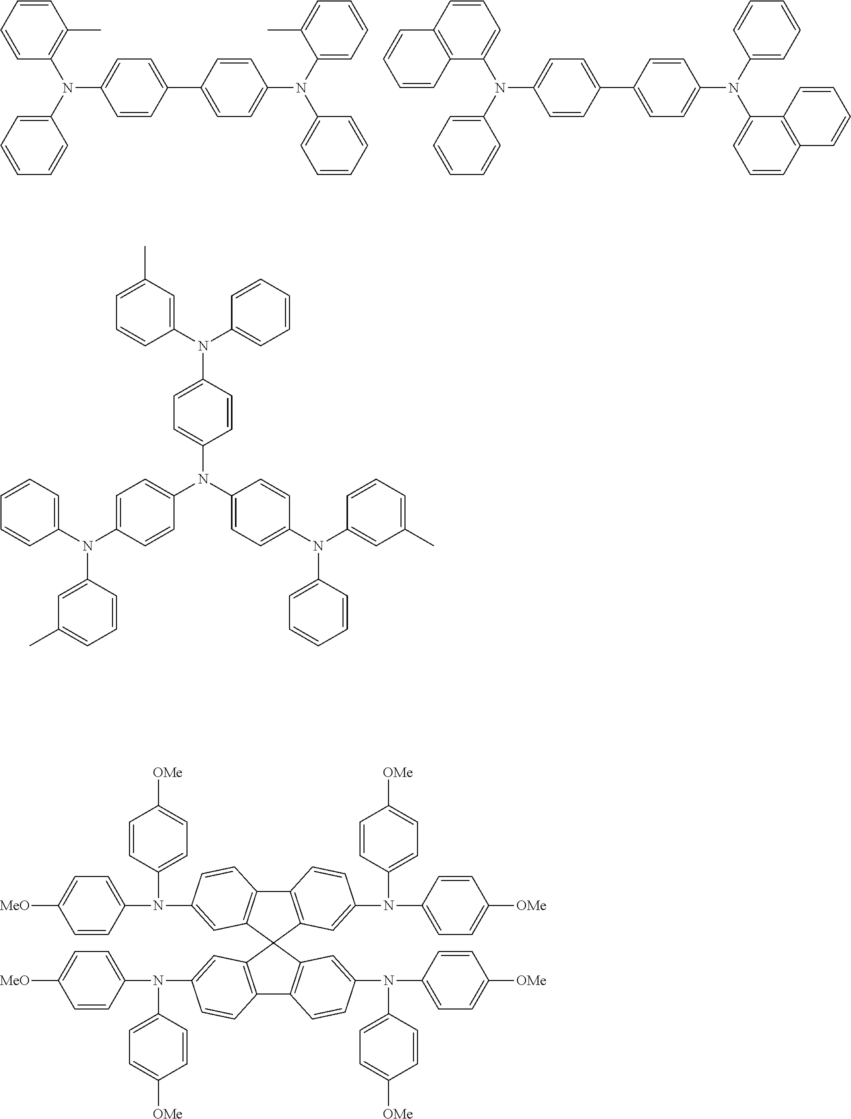

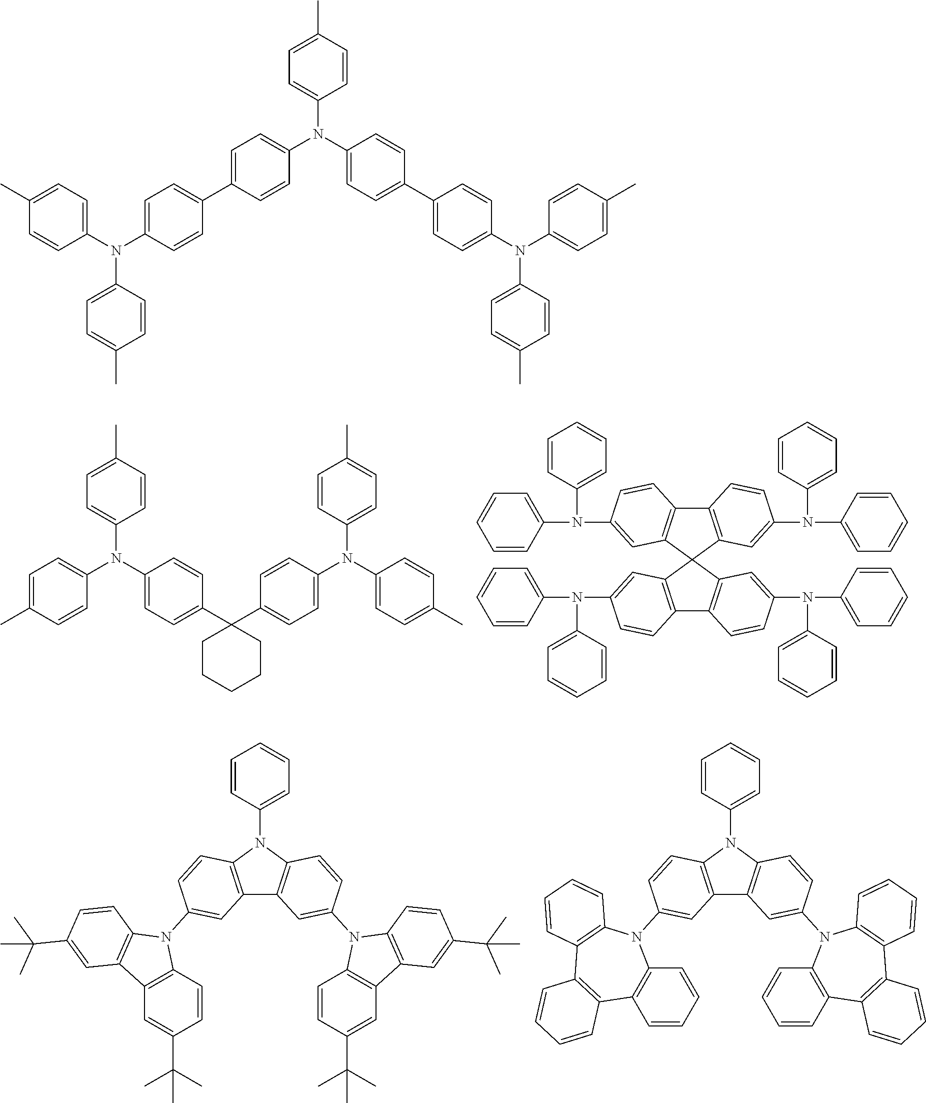

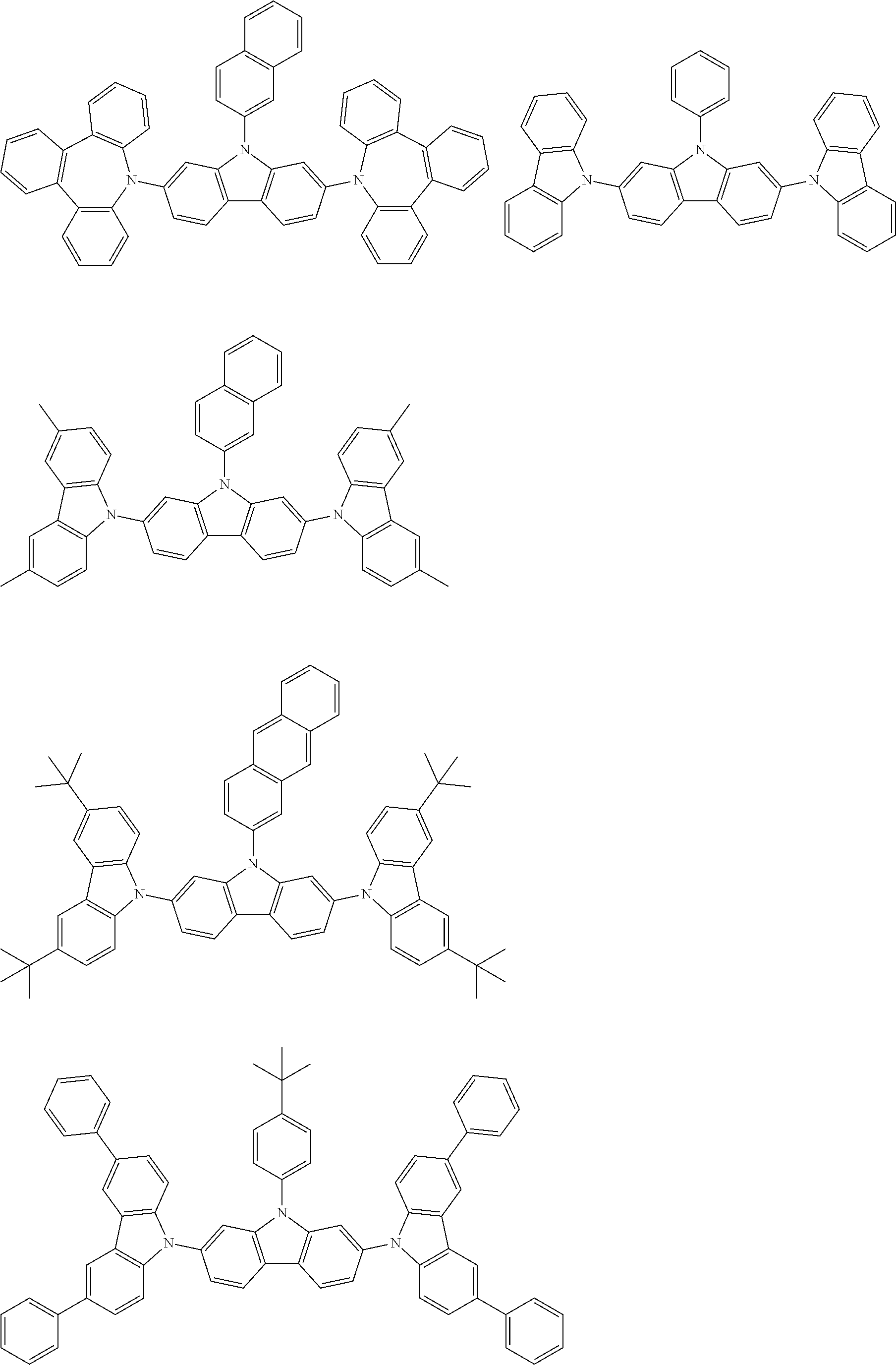

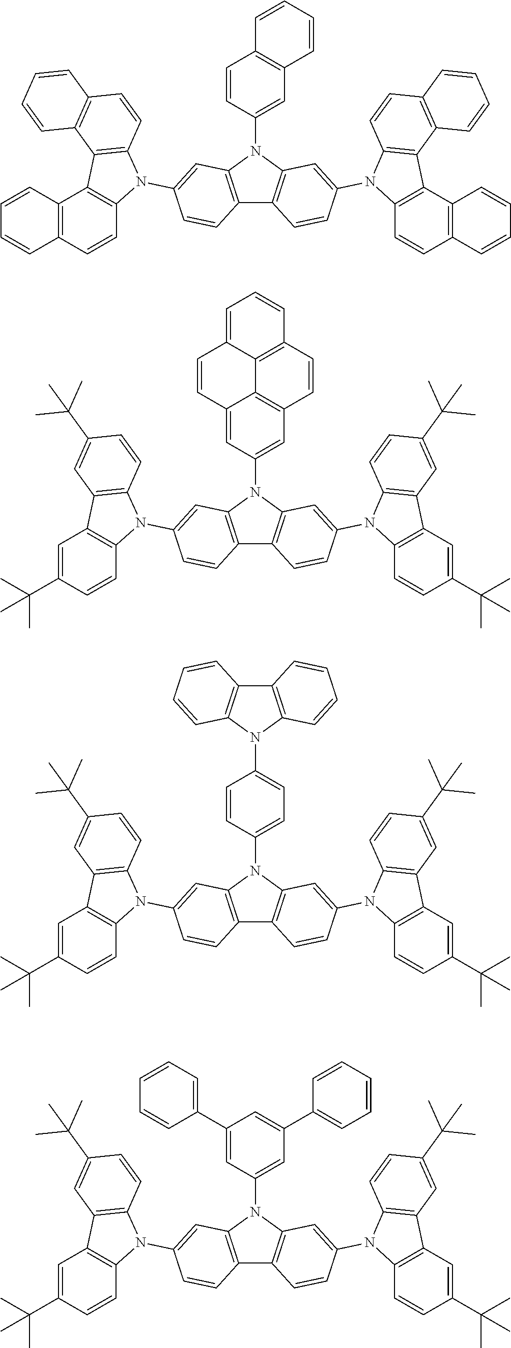

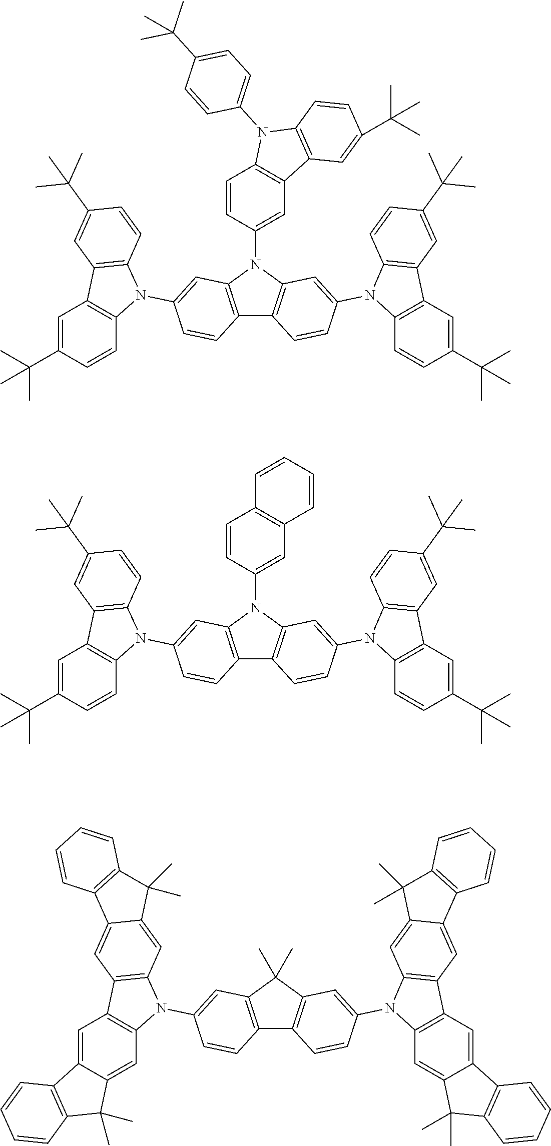

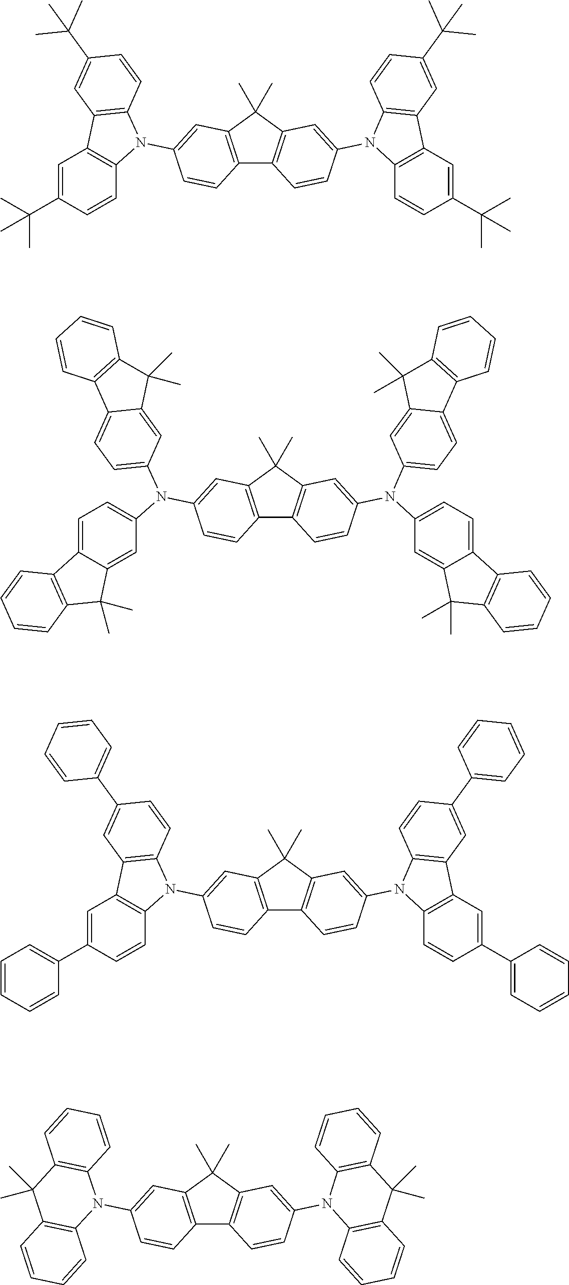

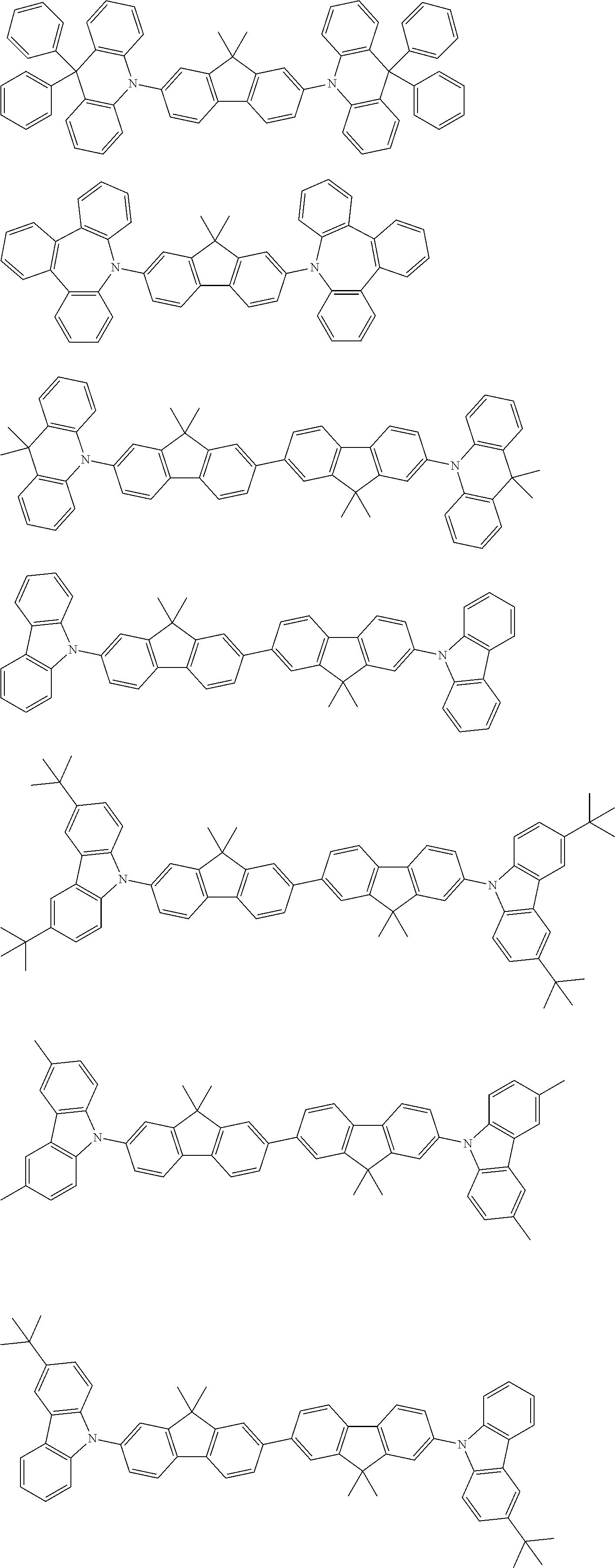

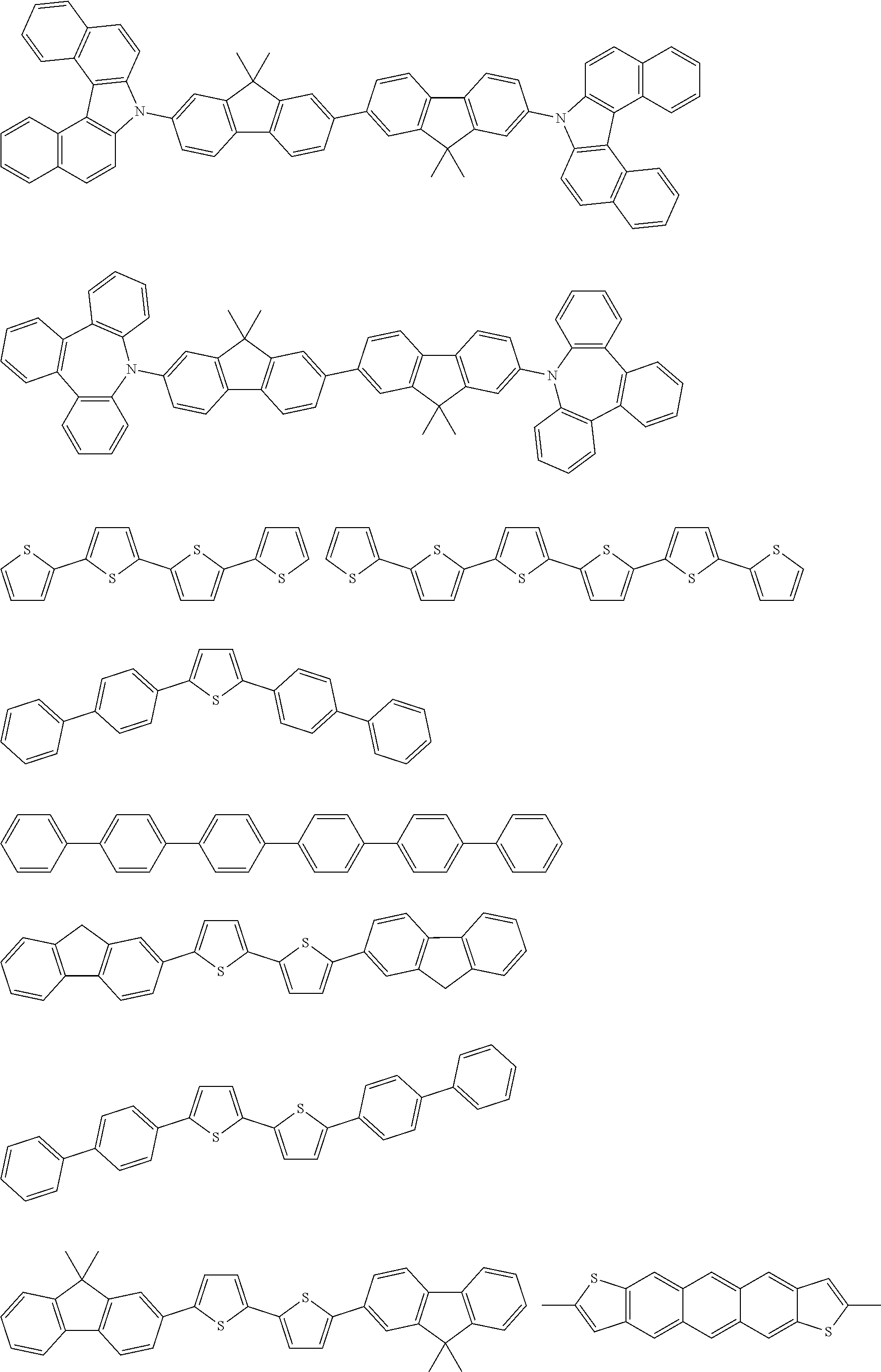

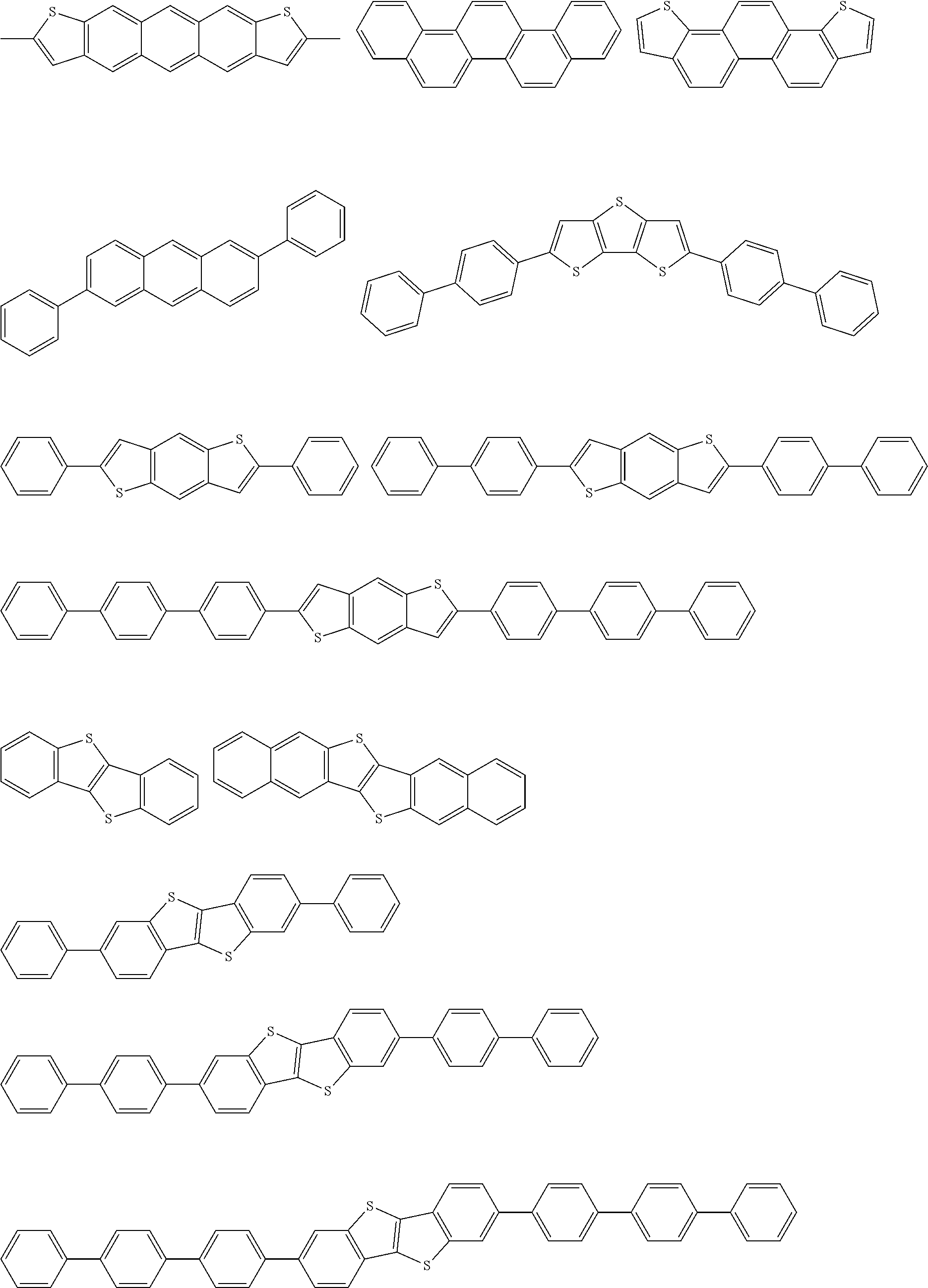

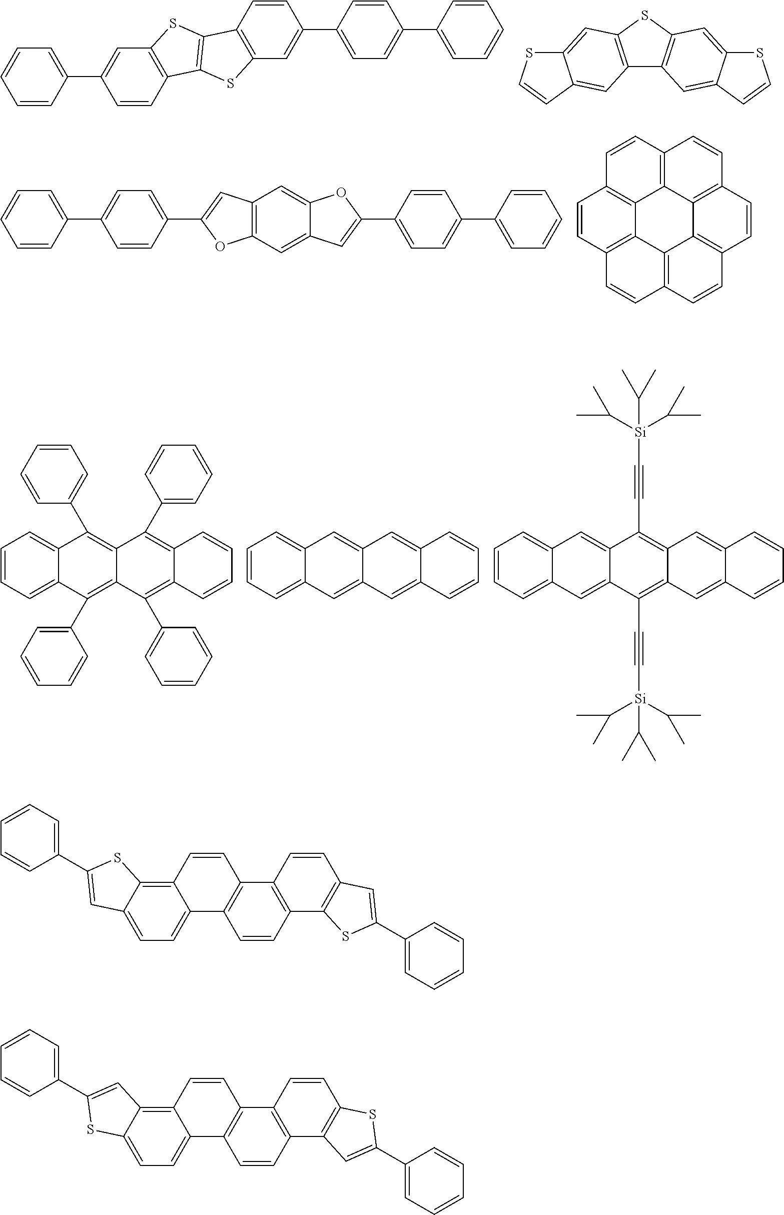

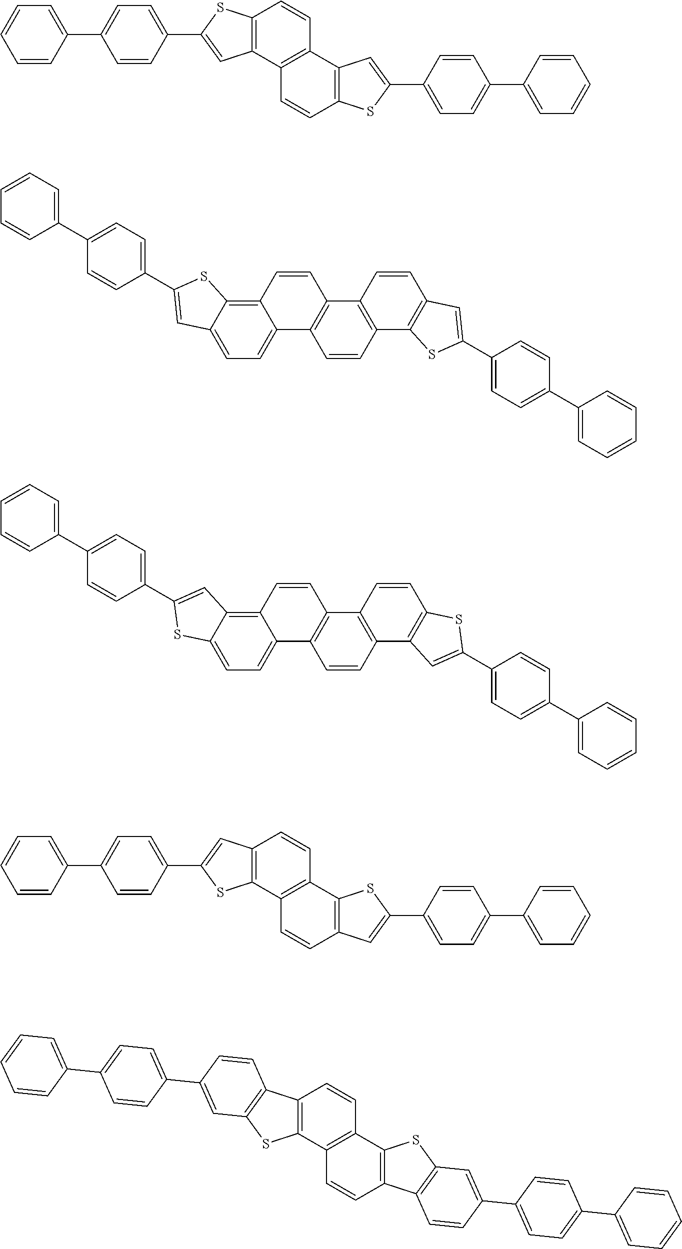 [SMILES: c1ccc(-c2ccc(-c3cc4c(ccc5c6cc(-c7ccc(-c8ccccc8)cc7)sc6ccc45)s3)cc2)cc1 ; c1ccc(-c2ccc(-c3cc4ccc5c6ccc7c(ccc8cc(-c9ccc(-c%10ccccc%10)cc9)sc87)c6ccc5c4s3)cc2)cc1 ; c1ccc(-c2ccc(-c3cc4c(ccc5c4ccc4c6ccc7sc(-c8ccc(-c9ccccc9)cc8)cc7c6ccc54)s3)cc2)cc1 ; c1ccc(-c2ccc(-c3cc4ccc5c(ccc6cc(-c7ccc(-c8ccccc8)cc7)sc65)c4s3)cc2)cc1 ; c1ccc(-c2ccc(-c3ccc4c(c3)sc3c4ccc4c3ccc3c5ccc(-c6ccc(-c7ccccc7)cc6)cc5sc34)cc2)cc1]

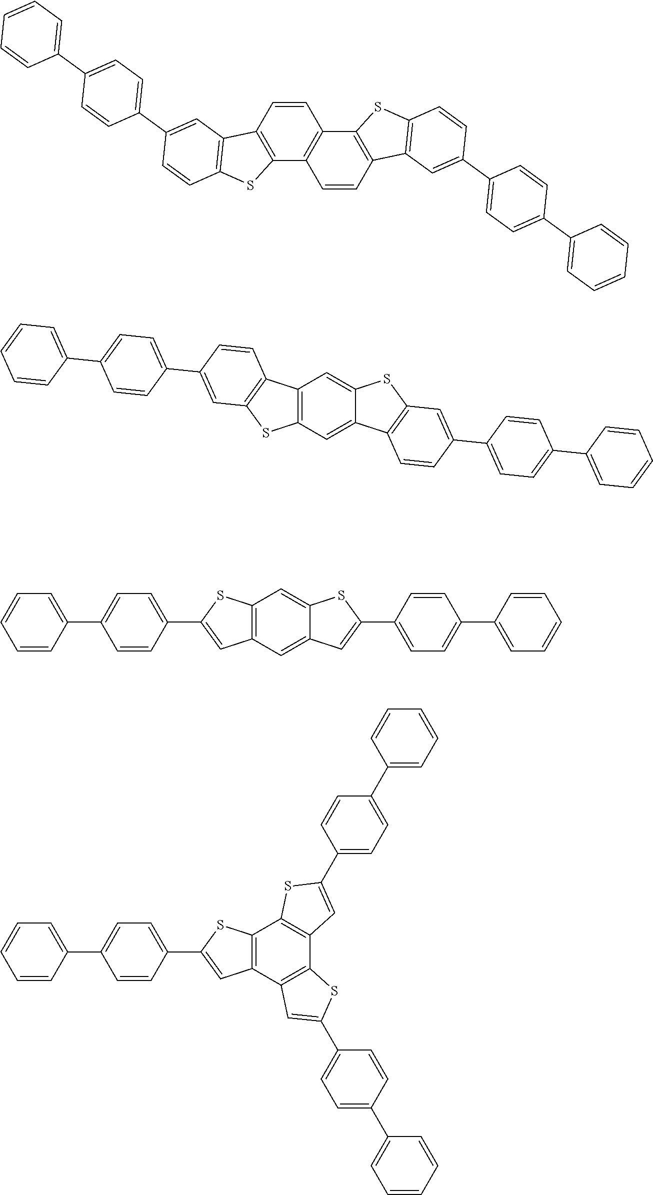 [SMILES: c1ccc(-c2ccc(-c3ccc4sc5c(ccc6c5ccc5c7cc(-c8ccc(-c9ccccc9)cc8)ccc7sc56)c4c3)cc2)cc1 ; c1ccc(-c2ccc(-c3ccc4c(c3)sc3cc5c(cc34)sc3cc(-c4ccc(-c6ccccc6)cc4)ccc35)cc2)cc1 ; c1ccc(-c2ccc(-c3cc4cc5cc(-c6ccc(-c7ccccc7)cc6)sc5cc4s3)cc2)cc1 ; c1ccc(-c2ccc(-c3cc4c5cc(-c6ccc(-c7ccccc7)cc6)sc5c5sc(-c6ccc(-c7ccccc7)cc6)cc5c4s3)cc2)cc1]

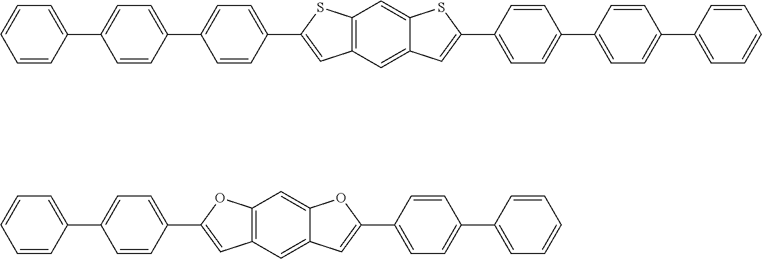 [SMILES: c1ccc(-c2ccc(-c3ccc(-c4cc5cc6cc(-c7ccc(-c8ccc(-c9ccccc9)cc8)cc7)sc6cc5s4)cc3)cc2)cc1 ; c1ccc(-c2ccc(-c3cc4cc5cc(-c6ccc(-c7ccccc7)cc6)oc5cc4o3)cc2)cc1]

The difference in the ionization potential between the specific compound and the p-type organic semiconductor is preferably 0.1 eV or more.

The p-type semiconductor material may be used alone, or two or more types thereof may be used in combination.

In a case where the photoelectric conversion film contains the p-type organic semiconductor, a content of the p-type organic semiconductor in the photoelectric conversion film (film thickness of p-type organic semiconductor in terms of single layer/film thickness of photoelectric conversion film×100) is preferably 15% to 75% by volume, more preferably 20% to 60% by volume, and still more preferably 25% to 50% by volume.

The photoelectric conversion film containing the specific compound is a non-light emitting film, and has a feature different from organic light emitting diodes (OLEDs). The non-light emitting film means a film having a light emission quantum efficiency of 1% or less, and the light emission quantum efficiency is preferably 0.5% or less, and more preferably 0.1% or less. The lower limit thereof is often 0% or more.

### <Film Formation Method>

Examples of a film formation method of the photoelectric conversion film include a dry film formation method.

Examples of the dry film formation method include a physical vapor deposition method such as a vapor deposition method (particularly, a vacuum vapor deposition method), a sputtering method, an ion plating method, and a molecular beam epitaxy (MBE) method, and a chemical vapor deposition (CVD) method such as plasma polymerization, and the vacuum vapor deposition method is preferable. In a case where the photoelectric conversion film is formed by the vacuum vapor deposition method, manufacturing conditions such as a degree of vacuum and a vapor deposition temperature can be set according to the normal method.

The film thickness of the photoelectric conversion film is preferably 10 to 1000 nm, more preferably 50 to 800 nm, and still more preferably 50 to 500 nm.

### [Electrode]

The photoelectric conversion element preferably has an electrode.

Electrodes (the upper electrode (the transparent conductive film) 15 and the lower electrode (the conductive film) 11) are formed of conductive materials. Examples of the conductive material include metals, alloys, metal oxides, electrically conductive compounds, and mixtures thereof.

Since light is incident through the upper electrode 15, the upper electrode 15 is preferably transparent to light to be detected. Examples of the materials constituting the upper electrode 15 include conductive metal oxides such as tin oxide (antimony tin oxide (ATO) and fluorine doped tin oxide (FTO)) doped with antimony, fluorine or the like, tin oxide, zinc oxide, indium oxide, indium tin oxide (ITO), and indium zinc oxide (IZO); metal thin films such as gold, silver, chromium, and nickel; mixtures or laminates of these metals and the conductive metal oxides; and organic conductive materials such as polyaniline, polythiophene, and polypyrrole; and nano carbon materials such as carbon nanotubes, graphene, and the like. From the viewpoint of high conductivity and transparency, conductive metal oxides are preferable.

Generally, in a case where the conductive film is made thinner than a certain range, the resistance value rapidly increases in many cases. In the solid-state imaging element in which the photoelectric conversion element according to the present embodiment is incorporated, the sheet resistance may be 100 to 10,000 Ω/□, and the degree of freedom of the film thickness range that can be reduced is large.

In addition, as the film thickness of the upper electrode (the transparent conductive film) 15 is thinner, the amount of light that the upper electrode absorbs is smaller, and the light transmittance usually increases. The increase in the light transmittance causes an increase in light absorbance in the photoelectric conversion film and an increase in the photoelectric conversion ability, which is preferable. Considering the suppression of leakage current, an increase in the resistance value of the thin film, and an increase in transmittance accompanied by the thinning, the thickness of the upper electrode 15 is preferably 5 to 100 nm, and more preferably 5 to 20 nm.

There is a case where the lower electrode 11 has transparency or an opposite case where the lower electrode 11 does not have transparency and reflects light, depending on the application. Examples of a material constituting the lower electrode 11 include conductive metal oxides such as tin oxide (ATO and FTO) doped with antimony, fluorine, or the like, tin oxide, zinc oxide, indium oxide, indium tin oxide (ITO), and indium zinc oxide (IZO); metals such as gold, silver, chromium, nickel, titanium, tungsten, and aluminum; conductive compounds (for example, titanium nitride (TiN)) such as oxides or nitrides of these metals; mixtures or laminates of these metals and conductive metal oxides; organic conductive materials such as polyaniline, polythiophene, and polypyrrole; and carbon materials such as carbon nanotubes and graphene.

The method of forming electrodes can be appropriately selected in accordance with the electrode material. Specific examples thereof include a wet method such as a printing method and a coating method; a physical method such as a vacuum vapor deposition method, a sputtering method, and an ion plating method; and a chemical method such as a CVD method and a plasma CVD method.

In a case where the material of the electrode is ITO, examples thereof include an electron beam method, a sputtering method, a resistance heating vapor deposition method, a chemical reaction method (such as a sol-gel method), and a coating method with a dispersion of indium tin oxide.

### [Charge Blocking Film: Electron Blocking Film and Positive Hole Blocking Film]

The photoelectric conversion element preferably includes one or more interlayers between the conductive film and the transparent conductive film, in addition to the photoelectric conversion film.

An example of the interlayer includes a charge blocking film. In a case where the photoelectric conversion element has this film, the characteristics (such as photoelectric conversion efficiency and response speed) of the photoelectric conversion element to be obtained are more excellent. Examples of the charge blocking film include an electron blocking film and a positive hole blocking film.

### <Electron Blocking Film>

The electron blocking film is a donor organic semiconductor material (a compound), and the p-type organic semiconductor described above can be used.

A polymer material can also be used as the electron blocking film.

Examples of the polymer material include a polymer such as phenylenevinylene, fluorene, carbazole, indole, pyrene, pyrrole, picoline, thiophene, acetylene, and diacetylene, and a derivative thereof.

The electron blocking film may be formed of a plurality of films.

The electron blocking film may be formed of an inorganic material. In general, since an inorganic material has a dielectric constant larger than that of an organic material, in a case where the inorganic material is used in the electron blocking film, a large voltage is applied to the photoelectric conversion film. Therefore, the photoelectric conversion efficiency increases. Examples of the inorganic material that can be used for the electron blocking film include calcium oxide, chromium oxide, copper chromium oxide, manganese oxide, cobalt oxide, nickel oxide, copper oxide, copper gallium oxide, copper strontium oxide, niobium oxide, molybdenum oxide, copper indium oxide, silver indium oxide, and iridium oxide.

### <Positive Hole Blocking Film>

A positive hole blocking film is an acceptor-property organic semiconductor material (a compound), and the n-type organic semiconductor described above can be used.

In addition, the positive hole blocking film may be formed with a plurality of films.

Examples of a method of producing a charge blocking film include a dry film formation method and a wet film formation method. Examples of the dry film formation method include a vapor deposition method and a sputtering method. The vapor deposition method may be any of a physical vapor deposition (PVD) method and a chemical vapor deposition (CVD) method, and the physical vapor deposition method such as a vacuum vapor deposition method is preferable. Examples of the wet film formation method include an ink jet method, a spray method, a nozzle printing method, a spin coating method, a dip coating method, a casting method, a die coating method, a roll coating method, a bar coating method, and a gravure coating method, and an ink jet method is preferable from the viewpoint of high accuracy patterning. Each film thickness of the charge blocking films (the electron blocking film and the positive hole blocking film) is preferably 3 to 200 nm, more preferably 5 to 100 nm, and still more preferably 5 to 30 nm.

### <Substrate>

The photoelectric conversion element may further include a substrate.

Examples of the substrate include a semiconductor substrate, a glass substrate, and a plastic substrate.

As a position of the substrate, in general, the conductive film, the photoelectric conversion film, and the transparent conductive film are laminated on the substrate in this order.

### <Sealing Layer>

The photoelectric conversion element may further include a sealing layer.

The performance of the photoelectric conversion material may deteriorate noticeably due to the presence of deterioration factors such as water molecules. The deterioration can be prevented by coating and sealing the entirety of the photoelectric conversion film with the sealing layer such as diamond-like carbon (DLC) or ceramics such as metal oxide, metal nitride, or metal nitride oxide which are dense and into which water molecules do not permeate.

Examples of the sealing layer include compounds described in paragraphs to of JP2011-082508A, the contents of which are incorporated herein by reference.

### [Imaging Element]

An example of the application of the photoelectric conversion element includes an imaging element.

The imaging element is an element that converts optical information of an image into an electric signal. In general, a plurality of the photoelectric conversion elements are arranged in a matrix on the same plane, and an optical signal is converted into an electric signal in each photoelectric conversion element (pixel) to sequentially output the electric signal to the outside of the imaging element for each pixel. Therefore, each pixel is formed of one or more photoelectric conversion elements and one or more transistors.

### [Optical Sensor]

Examples of another application of the photoelectric conversion element include the photoelectric cell and the optical sensor, but the photoelectric conversion element of the embodiment of the present invention is preferably used as the optical sensor. The photoelectric conversion element may be used alone as the optical sensor. Alternately, the photoelectric conversion element may be used as a line sensor in which the photoelectric conversion elements are linearly arranged or as a two-dimensional sensor in which the photoelectric conversion elements are arranged on a plane.

### [Compound]

The present invention further includes the invention of compounds. The compound according to the embodiment of the present invention is the specific compound.

### EXAMPLES

Hereinafter, the present invention will be described in more detail based on Examples. The material, the amount used, the proportion, the process contents, the process procedure, and the like shown in the following examples can be appropriately changed, within a range not departing from the gist of the present invention. Therefore, the scope of the present invention should not be construed as being limited to Examples shown below.

[Compound used for Photoelectric Conversion Film]

### Synthesis of Compound (D-3)

A compound (D-3) was synthesized according to the following scheme.

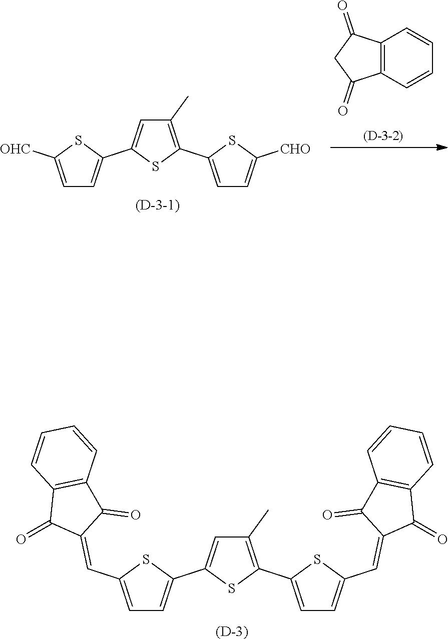

The compound (D-3-1) (2.0 mmol), the compound (D-3-2) (5.0 mmol), toluene (60 mL), and piperidine (0.02 mmol) were charged into a glass reaction container, and reacted at 100° C. for 2 hours under a nitrogen atmosphere. The precipitated solid was filtered, and the obtained solid was sequentially washed with tetrahydrofuran (THF), dimethylacetamide (DMAc), and tetrahydrofuran (THF), and then subjected to sublimation purification to obtain 1.0 mmol (yield: 50%) of a compound (D-3). The structure of the compound (D-3) was confirmed by LDI-MS.

LDI-MS (compound (D-3)): 574 (M⁺)

The compound used in the photoelectric conversion film other than the compound (D-3) was synthesized with reference to the synthesis method of the compound (D-3).

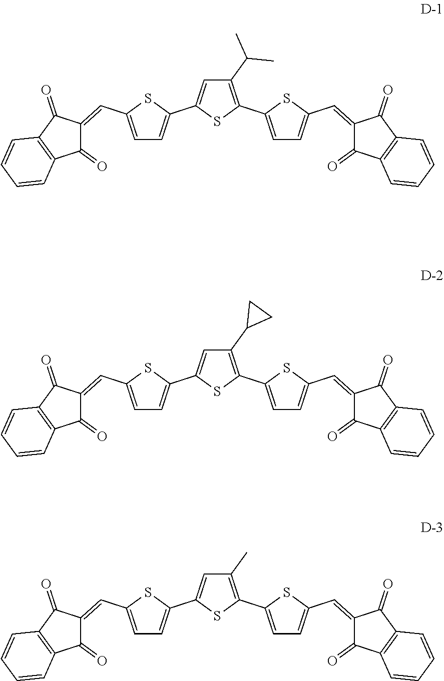 [SMILES: CC(C)c1cc(-c2ccc(C=C3C(=O)c4ccccc4C3=O)s2)sc1-c1ccc(C=C2C(=O)c3ccccc3C2=O)s1 ; O=C1C(=Cc2ccc(-c3cc(C4CC4)c(-c4ccc(C=C5C(=O)c6ccccc6C5=O)s4)s3)s2)C(=O)c2ccccc21 ; Cc1cc(-c2ccc(C=C3C(=O)c4ccccc4C3=O)s2)sc1-c1ccc(C=C2C(=O)c3ccccc3C2=O)s1]

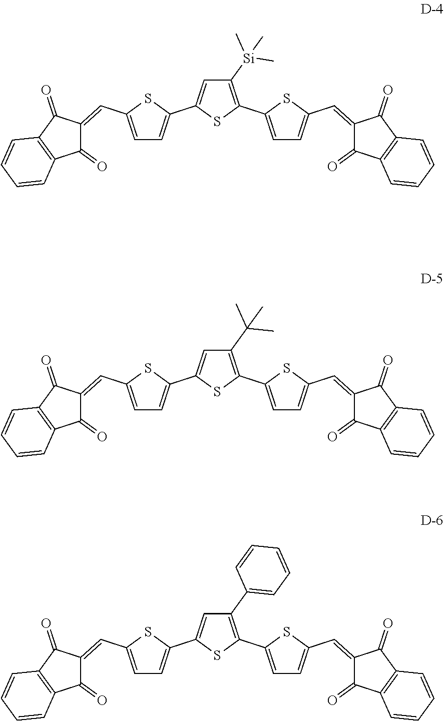 [SMILES: C[Si](C)(C)c1cc(-c2ccc(C=C3C(=O)c4ccccc4C3=O)s2)sc1-c1ccc(C=C2C(=O)c3ccccc3C2=O)s1 ; CC(C)(C)c1cc(-c2ccc(C=C3C(=O)c4ccccc4C3=O)s2)sc1-c1ccc(C=C2C(=O)c3ccccc3C2=O)s1 ; O=C1C(=Cc2ccc(-c3cc(-c4ccccc4)c(-c4ccc(C=C5C(=O)c6ccccc6C5=O)s4)s3)s2)C(=O)c2ccccc21]

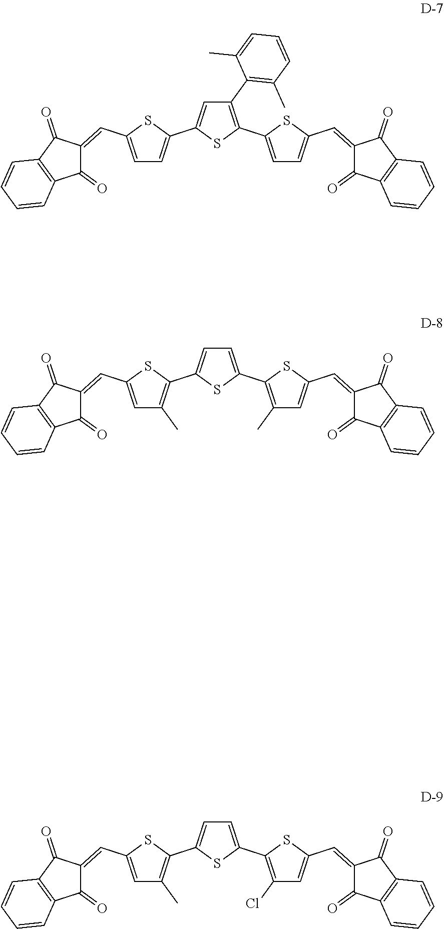 [SMILES: Cc1cccc(C)c1-c1cc(-c2ccc(C=C3C(=O)c4ccccc4C3=O)s2)sc1-c1ccc(C=C2C(=O)c3ccccc3C2=O)s1 ; Cc1cc(C=C2C(=O)c3ccccc3C2=O)sc1-c1ccc(-c2sc(C=C3C(=O)c4ccccc4C3=O)cc2C)s1 ; Cc1cc(C=C2C(=O)c3ccccc3C2=O)sc1-c1ccc(-c2sc(C=C3C(=O)c4ccccc4C3=O)cc2Cl)s1]

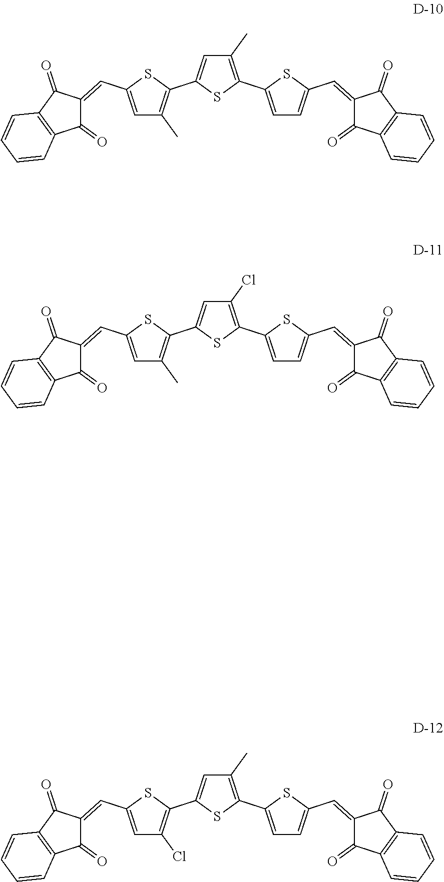 [SMILES: Cc1cc(C=C2C(=O)c3ccccc3C2=O)sc1-c1cc(C)c(-c2ccc(C=C3C(=O)c4ccccc4C3=O)s2)s1 ; Cc1cc(C=C2C(=O)c3ccccc3C2=O)sc1-c1cc(Cl)c(-c2ccc(C=C3C(=O)c4ccccc4C3=O)s2)s1 ; Cc1cc(-c2sc(C=C3C(=O)c4ccccc4C3=O)cc2Cl)sc1-c1ccc(C=C2C(=O)c3ccccc3C2=O)s1]

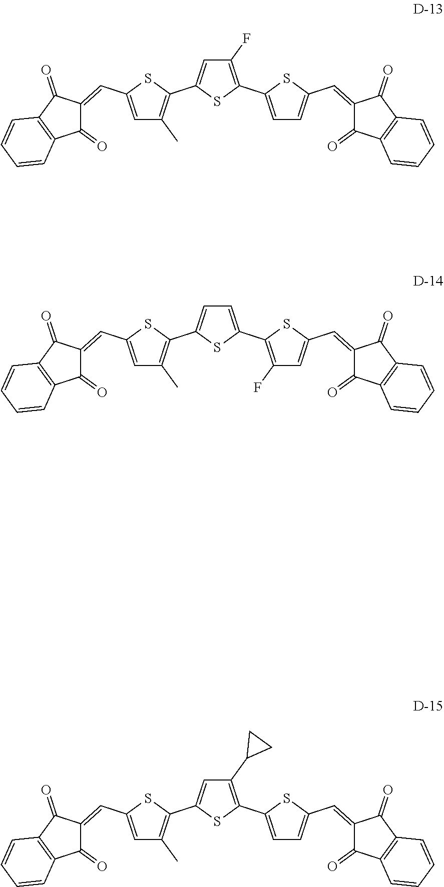 [SMILES: Cc1cc(C=C2C(=O)c3ccccc3C2=O)sc1-c1cc(F)c(-c2ccc(C=C3C(=O)c4ccccc4C3=O)s2)s1 ; Cc1cc(C=C2C(=O)c3ccccc3C2=O)sc1-c1ccc(-c2sc(C=C3C(=O)c4ccccc4C3=O)cc2F)s1 ; Cc1cc(C=C2C(=O)c3ccccc3C2=O)sc1-c1cc(C2CC2)c(-c2ccc(C=C3C(=O)c4ccccc4C3=O)s2)s1]

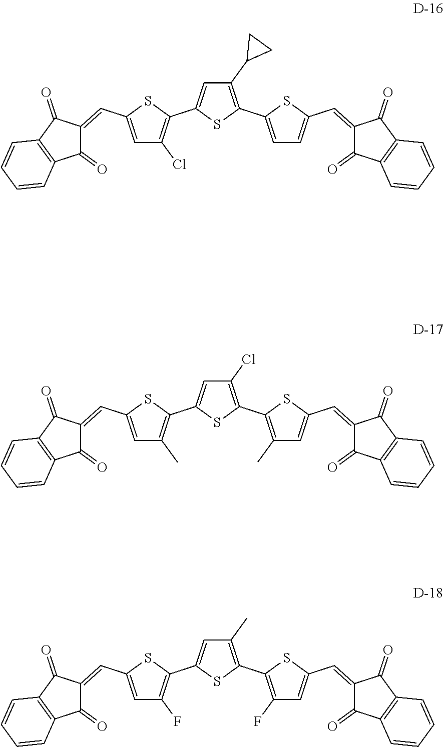 [SMILES: O=C1C(=Cc2ccc(-c3sc(-c4sc(C=C5C(=O)c6ccccc6C5=O)cc4Cl)cc3C3CC3)s2)C(=O)c2ccccc21 ; Cc1cc(C=C2C(=O)c3ccccc3C2=O)sc1-c1cc(Cl)c(-c2sc(C=C3C(=O)c4ccccc4C3=O)cc2C)s1 ; Cc1cc(-c2sc(C=C3C(=O)c4ccccc4C3=O)cc2F)sc1-c1sc(C=C2C(=O)c3ccccc3C2=O)cc1F]

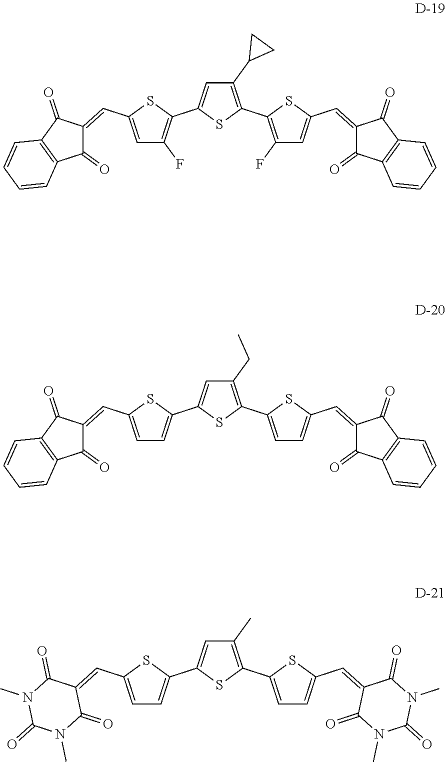

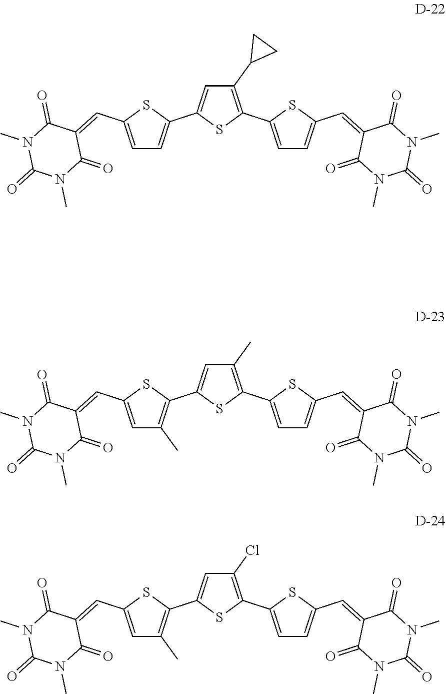

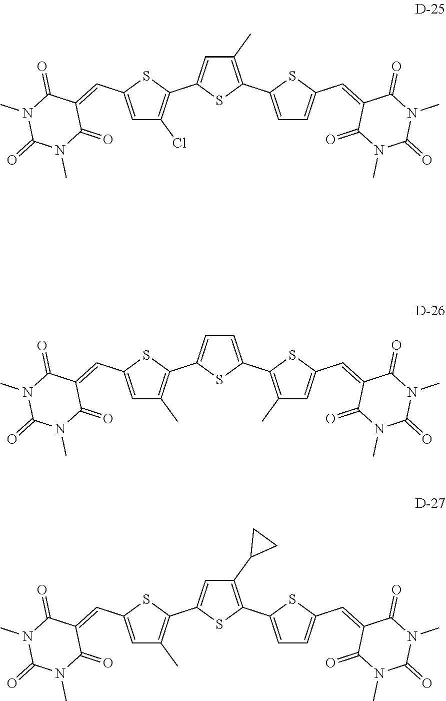

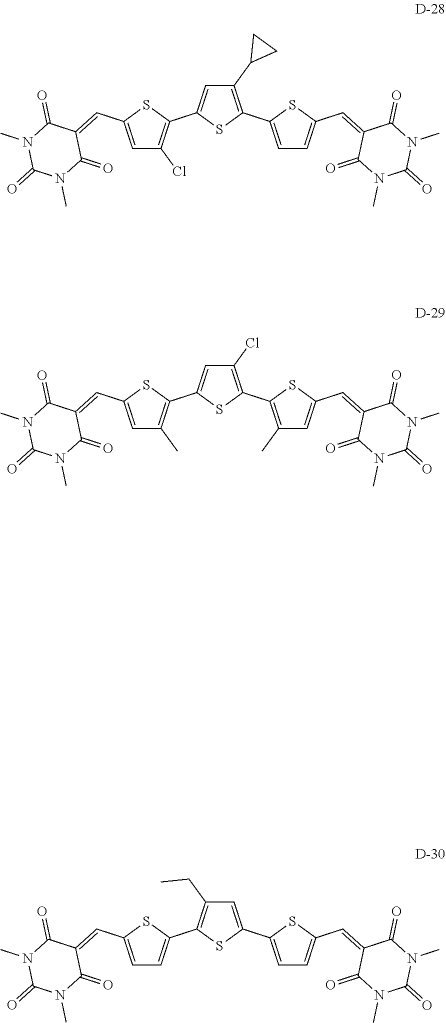

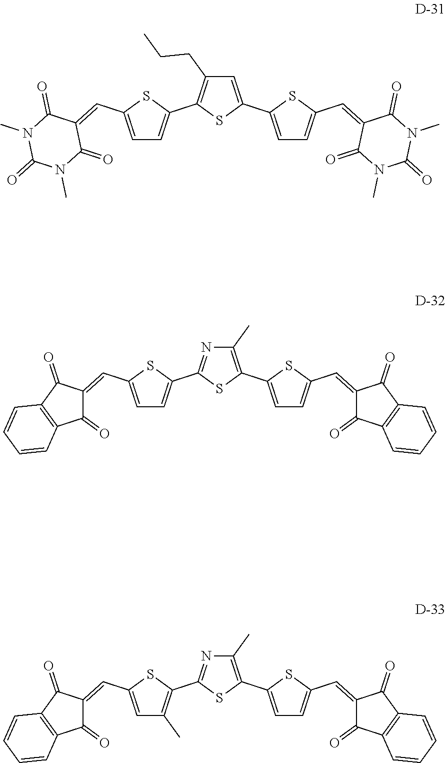

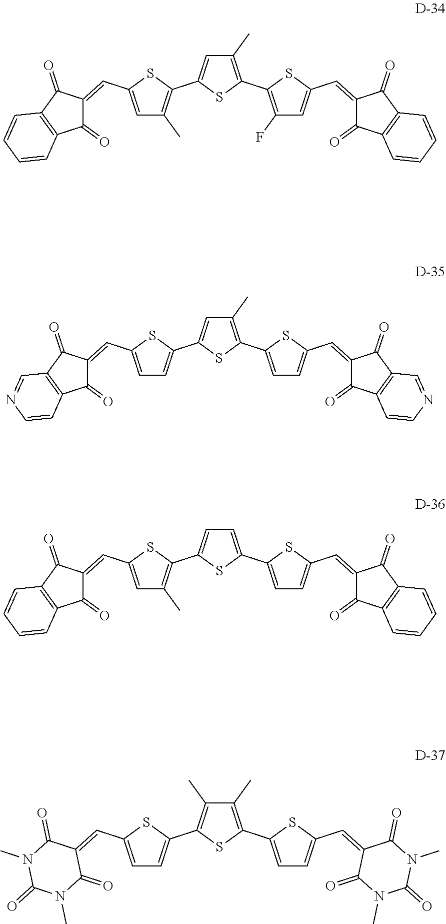

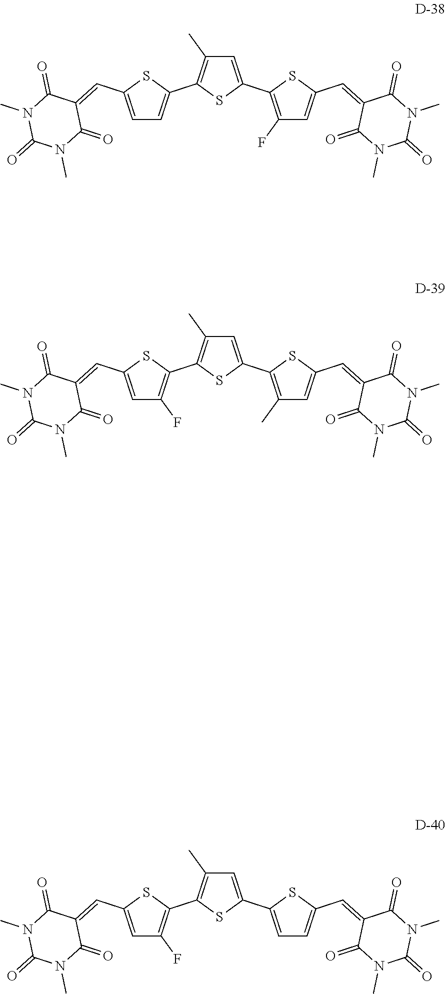

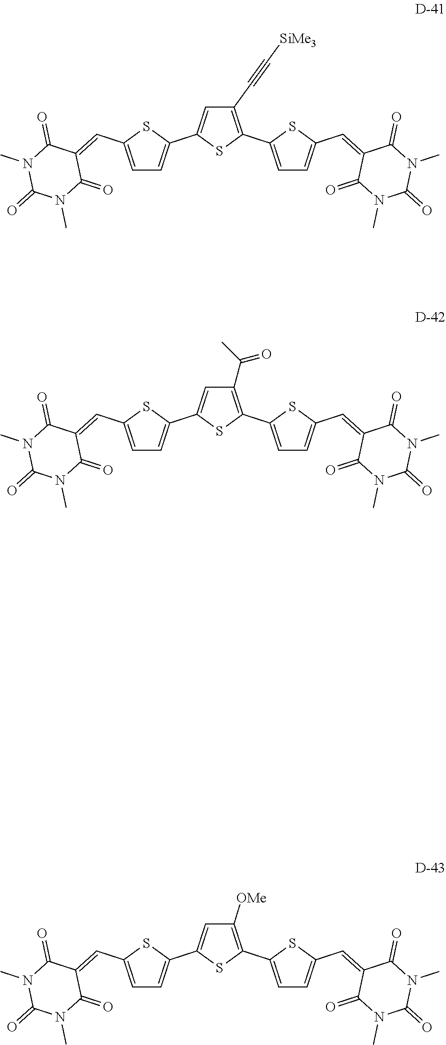

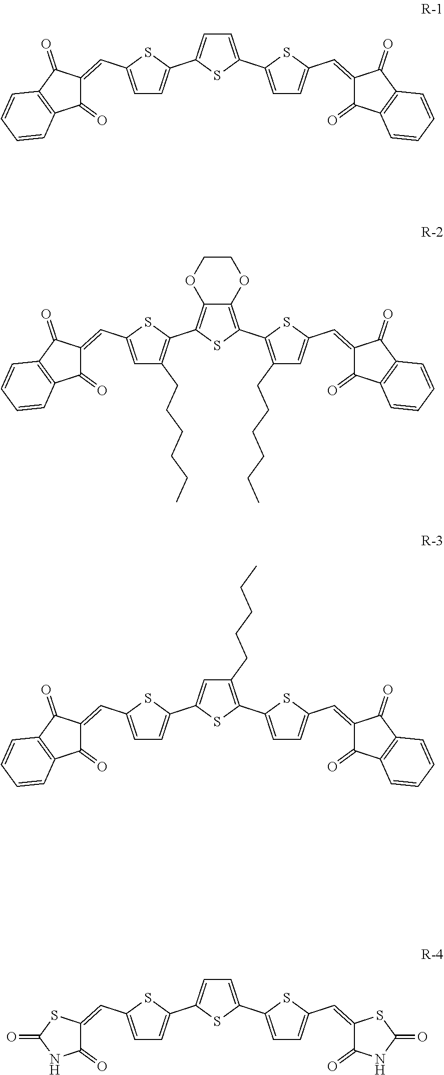 [SMILES: O=C1C(=Cc2ccc(-c3ccc(-c4ccc(C=C5C(=O)c6ccccc6C5=O)s4)s3)s2)C(=O)c2ccccc21 ; CCCCCCc1cc(C=C2C(=O)c3ccccc3C2=O)sc1-c1sc(-c2sc(C=C3C(=O)c4ccccc4C3=O)cc2CCCCCC)c2c1OCCO2 ; CCCCCc1cc(-c2ccc(C=C3C(=O)c4ccccc4C3=O)s2)sc1-c1ccc(C=C2C(=O)c3ccccc3C2=O)s1 ; O=C1NC(=O)/C(=C\c2ccc(-c3ccc(-c4ccc(/C=C5/SC(=O)NC5=O)s4)s3)s2)S1]

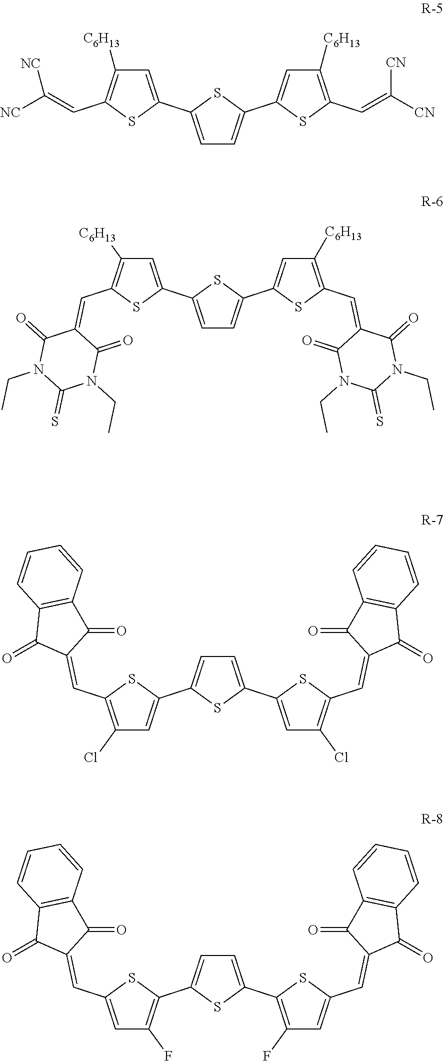

[n-Type Organic Semiconductor]
C60: fullerene (C₆₀)
   [p-Type Organic Semiconductor]

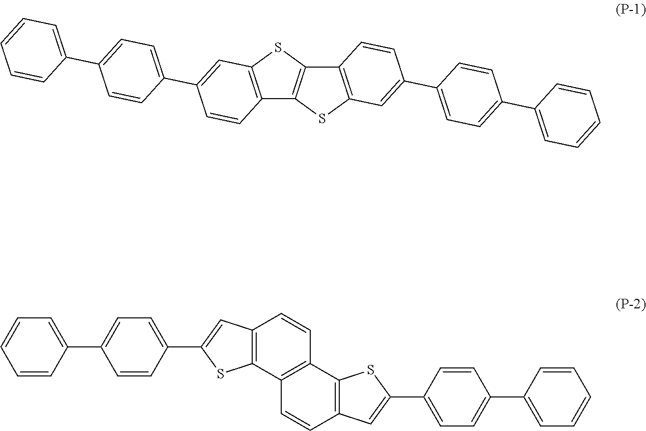 [SMILES: c1ccc(-c2ccc(-c3ccc4c(c3)sc3c5ccc(-c6ccc(-c7ccccc7)cc6)cc5sc43)cc2)cc1 ; c1ccc(-c2ccc(-c3cc4ccc5c(ccc6cc(-c7ccc(-c8ccccc8)cc7)sc65)c4s3)cc2)cc1]

### Evaluation

### [Production of Photoelectric Conversion Element]

The photoelectric conversion element (A) of the form illustrated in FIG. 1 was produced using the obtained compounds. Here, the photoelectric conversion element consists of a lower electrode 11, an electron blocking film 16A, a photoelectric conversion film 12, and an upper electrode 15.

Specifically, an amorphous ITO was formed into a film on a glass substrate by a sputtering method to form the lower electrode 11 (thickness: 30 nm). Furthermore, a compound (EB-1) was formed into a film on the lower electrode 11 by a vacuum thermal vapor deposition method to form the electron blocking film 16A (thickness: 30 nm).

Furthermore, in a state where the temperature of the glass substrate was controlled to 25° C., each specific compound, each compound for comparison, the n-type organic semiconductor (fullerene (C₆₀)), and the p-type organic semiconductor was co-vapor deposited on the electron blocking film 16A by a vacuum vapor deposition method, each to be 80 nm in terms of a single layer, thereby forming a film. As a result, the photoelectric conversion film 12 having a bulk hetero structure with a wavelength of 240 nm was formed. In this case, a film formation rate of the photoelectric conversion film 12 was set to 1.0 Å/sec. Furthermore, a compound (EB-2) was vapor-deposited on the photoelectric conversion film 12 to form the positive hole blocking film 16B (thickness: 10 nm). Amorphous ITO was formed into a film on the positive hole blocking film 16B by a sputtering method to form the upper electrode 15 (the transparent conductive film) (film thickness: 10 nm). After the SiO film was formed as the sealing layer on the upper electrode 15 by a vacuum vapor deposition method, an aluminum oxide (Al₂O₃) layer was formed thereon by an atomic layer chemical vapor deposition (ALCVD) method to produce each photoelectric conversion element and each photoelectric conversion element (A) was produced.

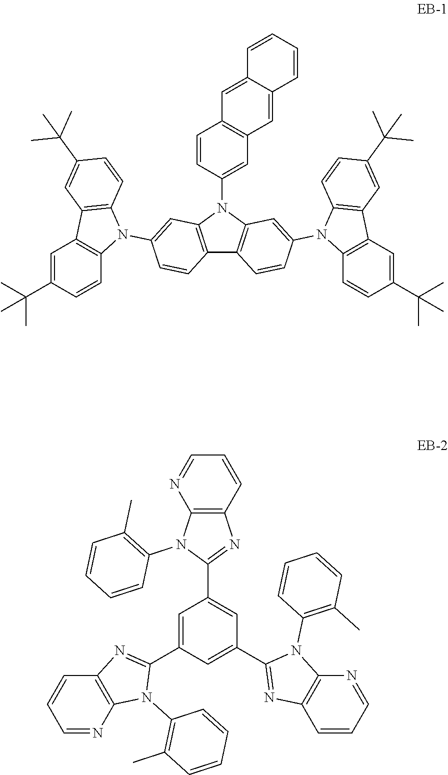 [SMILES: CC(C)(C)c1ccc2c(c1)c1cc(C(C)(C)C)ccc1n2-c1ccc2c3ccc(-n4c5ccc(C(C)(C)C)cc5c5cc(C(C)(C)C)ccc54)cc3n(-c3ccc4cc5ccccc5cc4c3)c2c1 ; Cc1ccccc1-n1c(-c2cc(-c3nc4cccnc4n3-c3ccccc3C)cc(-c3nc4cccnc4n3-c3ccccc3C)c2)nc2cccnc21]

### Evaluation of Photoelectric Conversion Efficiency (External Quantum Efficiency)

The drive of each photoelectric conversion element (A) thus obtained was confirmed. A voltage was applied to each photoelectric conversion element (A) to have an electric field strength of 2.0×10⁵ V/cm. Thereafter, light is emitted from the upper electrode (transparent conductive film) side to perform an incident photon-to-current conversion efficiency (IPCE) measurement, and an integrated value of the photoelectric conversion efficiency (external quantum efficiency) in wavelength range of 450 nm to 700 nm was calculated. The photoelectric conversion efficiency was measured using a constant energy quantum efficiency measuring device manufactured by Optel Co., Ltd. The amount of light emitted was 50 μW/cm². In a case where the integrated value of the photoelectric conversion efficiency of the photoelectric conversion element (A) of Example 1 was standardized to 1, the integrated value of the photoelectric conversion efficiency of each photoelectric conversion element (A) was obtained and evaluated according to the following evaluation standard.
A: 1.4 or more
B: 1.2 or more and less than 1.4
C: 1.0 or more and less than 1.2
D: 0.8 or more and less than 1.0
E: less than 0.8

### Evaluation of Manufacturing Suitability

A photoelectric conversion element (B) of each of Examples or each of Comparative Examples was produced according to the same procedure as that of the photoelectric conversion element (A), except that a film formation rate of the photoelectric conversion film 12 was set to 3.0 Å/sec. The photoelectric conversion efficiency (external quantum efficiency) of the obtained photoelectric conversion element (B) was evaluated by the same method as shown in the section of [Evaluation of photoelectric conversion efficiency (external quantum efficiency)].

The photoelectric conversion efficiencies of the photoelectric conversion element (A) and the photoelectric conversion element (B) in the same configuration of Examples or Comparative Examples were compared to calculate a relative ratio B/A of “the photoelectric conversion efficiency of the photoelectric conversion element (B)/the photoelectric conversion efficiency of the photoelectric conversion element (A)”. The manufacturing suitability of each photoelectric conversion element was evaluated by comparing the obtained values with the following standard. The compound having excellent evaluation results indicates that the compound is a material in which the performance is less likely to be deteriorated during high-speed film formation, and indicates that the compound has excellent manufacturing suitability. C or more is preferable, and A is more preferable.
A: relative ratio B/A of 0.90 or more
B: relative ratio B/A of 0.85 or more and less than 0.90
C: relative ratio B/A of 0.80 or more and less than 0.85
D: relative ratio B/A of 0.75 or more and less than 0.80
E: relative ratio B/A of less than 0.75

### Evaluation of Responsiveness

The responsiveness of each of the obtained photoelectric conversion elements (A) was evaluated.

A voltage was applied to each photoelectric conversion element to have a strength of 2.0×10⁵ V/cm. Thereafter, a green light emitting diode (LED) was turned on momentarily to emit light from the upper electrode (transparent conductive film) side, a photocurrent was measured with an oscilloscope, and a rise time until rising from a signal intensity of 0% to a signal intensity of 97% was calculated. Next, the rise time of the photoelectric conversion element (A) in a case of using the compound (D-1) was standardized to 1, and a relative value of the rise time of each photoelectric conversion element (A) to the rise time of the photoelectric conversion element (A) in a case of using the compound (D-1) (rise time of each photoelectric conversion element (A)/rise time of the photoelectric conversion element (A) in a case of using the compound (D-1)) was obtained, and the obtained value was evaluated according to the following standard. C or more is preferable, and A is more preferable.
A: less than 0.5
B: 0.5 or more and less than 1.0
C: 1.0 or more and less than 1.5
D: 1.5 or more and less than 2.5
E: 2.5 or more

Hereinafter, the evaluation results are shown in the table.

In the tables, each notation indicates the following.
“Formulae (2) to (8)” indicate whether or not the compound corresponds to the compound represented by any of Formulae (2) to (8).
“R^{Y1}” represents a substituent other than the group represented by R² among the substituents represented by RYl in Formula (1). “Cl” represents a chlorine atom, and “F” represents a fluorine atom.
“R^{Y2}” represents the group represented by R^{Y2} in Formula (1).

TABLE 1

| | Compound | | | | n-type organic | p-type organic | Photoelectric | | |
|---|---|---|---|---|---|---|---|---|---|
| | | Formulae | | | semiconductor | semiconductor | conversion | Manufacturing | Respon- |
| | Type | (2) to (8) | R^{Y1} | R^{Y2} | Type | Type | efficiency | suitability | siveness |
| Example 1 | (D-1) | Formula (2) | — | Isopropyl group | C₆₀ | (P-1) | C | B | C |
| Example 2 | (D-1) | Formula (2) | — | Isopropyl group | C₆₀ | (P-2) | C | B | C |
| Example 3 | (D-2) | Formula (2) | — | Cyclopropyl group | C₆₀ | (P-1) | A | A | A |
| Example 4 | (D-3) | Formula (2) | — | Methyl group | C₆₀ | (P-1) | A | A | A |
| Example 5 | (D-4) | Formula (2) | — | Trimethylsilyl | C₆₀ | (P-1) | C | B | B |
| | | | | group | | | | | |
| Example 6 | (D-5) | Formula (2) | — | tert-Butyl group | C₆₀ | (P-1) | C | B | B |
| Example 7 | (D-6) | Formula (2) | — | Phenyl group | C₆₀ | (P-1) | A | A | A |
| Example 8 | (D-7) | Formula (2) | — | Dimethyl group | C₆₀ | (P-1) | A | A | A |
| Example 9 | (D-8) | Formula (4) | — | Methyl group/ | C₆₀ | (P-1) | A | A | A |
| | | | | Methyl group | | | | | |
| Example 10 | (D-9) | Formula (3) | Cl | Methyl group | C₆₀ | (P-1) | A | A | A |
| Example 11 | (D-10) | Formula (5) | — | Methyl group/ | C₆₀ | (P-1) | A | A | A |
| | | | | Methyl group | | | | | |
| Example 12 | (D-11) | Formula (3) | Cl | Methyl group | C₆₀ | (P-1) | A | A | A |
| Example 13 | (D-12) | Formula (2) | Cl | Methyl group | C₆₀ | (P-1) | A | A | A |
| Example 14 | (D-13) | Formula (3) | F | Methyl group | C₆₀ | (P-1) | A | A | A |
| Example 15 | (D-14) | Formula (3) | F | Methyl group | C₆₀ | (P-1) | A | A | A |
| Example 16 | (D-15) | Formula (5) | — | Methyl group/ | C₆₀ | (P-1) | A | A | A |
| | | | | cyclopropyl group | | | | | |
| Example 17 | (D-16) | Formula (2) | Cl | Cyclopropyl group | C₆₀ | (P-1) | A | A | A |
| Example 18 | (D-17) | Formula (4) | Cl | Methyl group/ | C₆₀ | (P-1) | B | B | B |
| | | | | Methyl group | | | | | |
| Example 19 | (D-18) | Formula (2) | FF | Methyl group | C₆₀ | (P-1) | A | A | A |
| Example 20 | (D-19) | Formula (2) | FF | Cyclopropyl group | C₆₀ | (P-1) | A | A | A |
| Example 21 | (D-20) | Formula (2) | — | Ethyl group | C₆₀ | (P-1) | A | A | A |
| Example 22 | (D-21) | Formula (2) | — | Methyl group | C₆₀ | (P-1) | A | A | A |
| Example 23 | (D-22) | Formula (2) | — | Cyclopropyl group | C₆₀ | (P-1) | A | A | A |
| Example 24 | (D-23) | Formula (5) | — | Methyl group/ | C₆₀ | (P-1) | A | A | A |
| | | | | Methyl group | | | | | |
| Example 25 | (D-24) | Formula (3) | Cl | Methyl group | C₆₀ | (P-1) | A | A | A |
| Example 26 | (D-25) | Formula (2) | Cl | Methyl group | C₆₀ | (P-1) | A | A | A |
| Example 27 | (D-26) | Formula (4) | — | Methyl group/ | C₆₀ | (P-1) | A | A | A |
| | | | | Methyl group | | | | | |
| Example 28 | (D-27) | Formula (5) | — | Methyl group/ | C₆₀ | (P-1) | A | A | A |
| | | | | cyclopropyl group | | | | | |
| Example 29 | (D-28) | Formula (2) | CI | Cyclopropyl group | C₆₀ | (P-1) | A | A | A |
| Example 30 | (D-29) | Formula (4) | CI | Methyl group/ | C₆₀ | (P-1) | B | B | B |
| | | | | Methyl group | | | | | |
| Example 31 | (D-30) | Formula (2) | — | Ethyl group | C₆₀ | (P-1) | A | A | A |
| Example 32 | (D-31) | Formula (2) | — | n-Propyl group | C₆₀ | (P-1) | B | C | C |
| Example 33 | (D-32) | Formula (2) | — | Methyl group | C₆₀ | (P-1) | A | A | A |
| Example 34 | (D-33) | Formula (5) | — | Methyl group/ | C₆₀ | (P-1) | A | A | A |
| | | | | Methyl group | | | | | |
| Example 35 | (D-34) | Formula (5) | F | Methyl group/ | C₆₀ | (P-1) | A | A | A |
| | | | | Methyl group | | | | | |
| Example 36 | (D-35) | Formula (2) | — | Methyl group | C₆₀ | (P-1) | A | A | A |
| Example 37 | (D-36) | Formula (3) | — | Methyl group | C₆₀ | (P-1) | A | A | A |
| Example 38 | (D-37) | Formula (8) | — | Methyl group/ | C₆₀ | (P-1) | A | A | A |
| | | | | Methyl group | | | | | |
| Example 39 | (D-38) | Formula (2) | F | Methyl group | C₆₀ | (P-1) | A | A | A |
| Example 40 | (D-39) | Formula (5) | F | Methyl group | C₆₀ | (P-1) | A | A | A |
| Example 41 | (D-40) | Formula (2) | F | Methyl group | C₆₀ | (P-1) | A | A | A |
| Example 42 | (D-41) | Formula (2) | — | Trimethyl- | C₆₀ | (P-1) | B | B | B |
| | | | | silylacetylene | | | | | |
| | | | | group | | | | | |
| Example 43 | (D-42) | Formula (2) | — | Acyl group | C₆₀ | (P-1) | B | B | B |
| Example 44 | (D-43) | Formula (2) | — | Methoxy group | C₆₀ | (P-1) | A | B | B |
| Comparative | (R-1) | — | — | — | C₆₀ | (P-1) | C | D | C |
| Example 1 | | | | | | | | | |
| Comparative | (R-2) | — | — | — | C₆₀ | (P-1) | E | E | E |
| Example 2 | | | | | | | | | |
| Comparative | (R-3) | — | — | — | C₆₀ | (P-1) | D | E | D |
| Example 3 | | | | | | | | | |
| Comparative | (R-4) | — | — | — | C₆₀ | (P-1) | E | E | E |
| Example 4 | | | | | | | | | |
| Comparative | (R-5) | — | — | — | C₆₀ | (P-1) | E | E | E |
| Example 5 | | | | | | | | | |
| Comparative | (R-6) | — | — | — | C₆₀ | (P-1) | E | E | E |
| Example 6 | | | | | | | | | |
| Comparative | (R-7) | — | — | — | C₆₀ | (P-1) | C | D | C |
| Example 7 | | | | | | | | | |
| Comparative | (R-8) | — | — | — | C₆₀ | (P-1) | C | D | C |
| Example 8 | | | | | | | | | |

From the results shown in the table, it was confirmed that the desired effects were obtained in the photoelectric conversion element according to the embodiment of the present invention.

In a case where R^{Y2} represents a methyl group, an ethyl group, a tert-butyl group, a cyclopropyl group, a cyclobutyl group, a cyclopentyl group, a cyclohexyl group, a trimethylsilylacetylene group, an acyl group, a methoxy group, a phenyl group having 10 or less carbon atoms which may have a substituent Y, or a trimethylsilyl group, it was confirmed that the effect of the present invention was more excellent (Examples 1 to 8, 32, 33, and 39 to 44).

In Formula (1), in a case where the total number of the substituents represented by R^{Y1} and the groups represented by R^{Y2} among Y¹ to Y⁶ is 3 to 6, the substituent represented by R^{Y1} represents at least a halogen atom, and the halogen atom is a halogen atom other than chlorine atom, it was confirmed that the effect of the present invention was more excellent (Examples 18 to 20, 30, and 35).

Using any of the compound (D-7), the compound (D-21), and the compound (D-22), any of the coloring agents (B-1) to (B-10), a fullerene (C₆₀), and the p-type organic semiconductor (P-1), each single layer was formed such that specific compound: coloring agent: n-type organic semiconductor: p-type organic semiconductor=1:1:2:2, to form a photoelectric conversion film. Other procedures were performed in the same procedures as in the production of the photoelectric conversion element described above to produce each photoelectric conversion element, and each of the above-described evaluations was performed. A photoelectric conversion element was produced for all combinations of the specific compound and the coloring agent, and each evaluation was performed.

As a result, even in a case of using the coloring agent, the same results as the results of using the compound (D-7) shown in Table 1 were shown in a case of using the compound (D-7), the same results as the results of using the compound (D-21) shown in Table 1 were shown in a case of using the compound (D-21), and the same results as the results of using the compound (D-22) shown in Table 1 were shown in a case of using the compound (D-22).

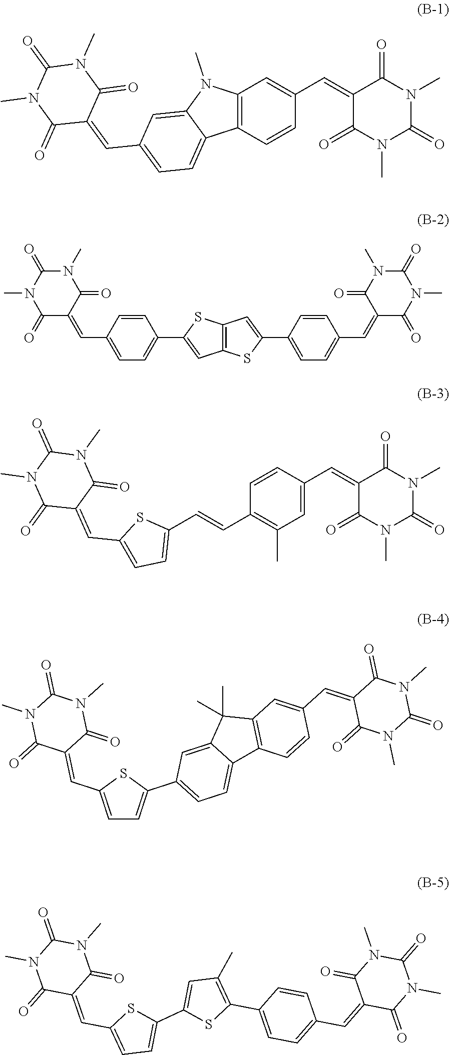

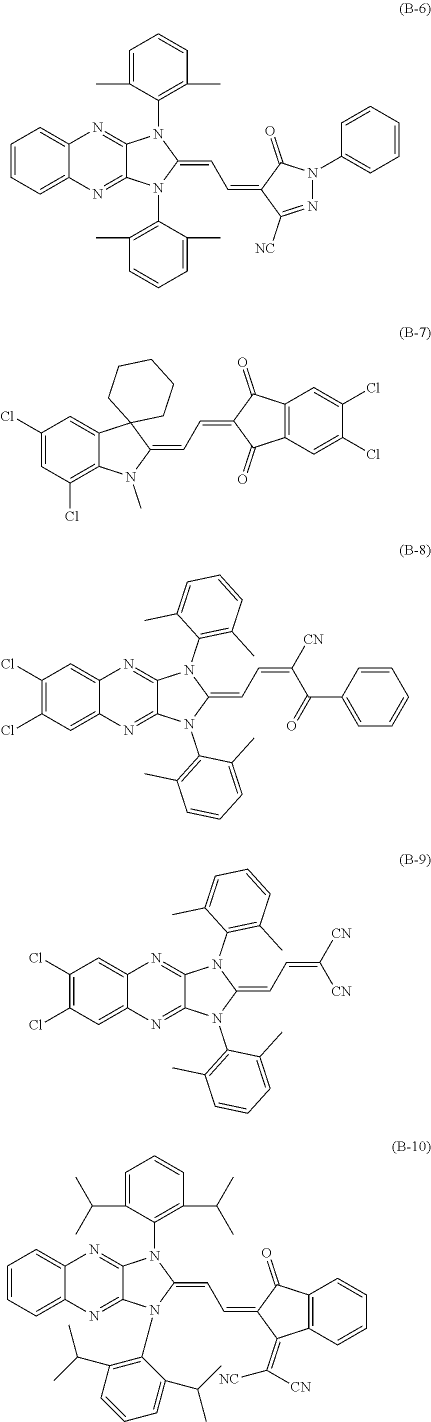 [SMILES: Cc1cccc(C)c1N1C(=C/C=C2\C(=O)N(c3ccccc3)N=C2C#N)N(c2c(C)cccc2C)c2nc3ccccc3nc21 ; CN1/C(=C/C=C2C(=O)c3cc(Cl)c(Cl)cc3C2=O)C2(CCCCC2)c2cc(Cl)cc(Cl)c21 ; Cc1cccc(C)c1N1C(=C/C=C(/C#N)C(=O)c2ccccc2)N(c2c(C)cccc2C)c2nc3cc(Cl)c(Cl)cc3nc21 ; Cc1cccc(C)c1N1C(=CC=C(C#N)C#N)N(c2c(C)cccc2C)c2nc3cc(Cl)c(Cl)cc3nc21 ; CC(C)c1cccc(C(C)C)c1N1C(=C/C=C2\C(=O)c3ccccc3C2=C(C#N)C#N)N(c2c(C(C)C)cccc2C(C)C)c2nc3ccccc3nc21]

### EXPLANATION OF REFERENCES

10a, 10b: photoelectric conversion element
11: conductive film (lower electrode)
12: photoelectric conversion film
15: transparent conductive film (upper electrode)
16A: electron blocking film
16B: positive hole blocking film

What is claimed is:

1. A photoelectric conversion element comprising, in the following order:
a conductive film;
a photoelectric conversion film; and
a transparent conductive film,
wherein the photoelectric conversion film contains a compound represented by Formula (1), 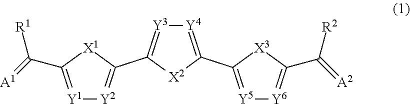
in Formula (1), Y¹to Y⁶ each independently represent —CR^{Y1}═ or —N═, R^{Y1} represents a hydrogen atom or a substituent, at least one of Y¹, Y², Y³, Y⁴, Y⁵, or Y⁶ represents —CR^{Y2}═, R^{Y2} represents a methyl group which may have a halogen atom, an ethyl group which may have a halogen atom, a linear propyl group which may have a halogen atom, an aliphatic hydrocarbon group having 3 to 6 carbon atoms, which has a branched structure and may have a halogen atom, an aliphatic hydrocarbon group which has a cyclic structure and may have a halogen atom, an acyl group which may have a halogen atom, —C≡C—Si(R)₃, an aromatic ring group which may have a substituent Y, an aliphatic heterocyclic group which may have a substituent Y, or —Si(R)₃,
the methyl group which may have a halogen atom, the ethyl group which may have a halogen atom, the linear propyl group which may have a halogen atom, the aliphatic hydrocarbon group having 3 to 6 carbon atoms, which has a branched structure and may have a halogen atom, and the aliphatic hydrocarbon group which has a cyclic structure and may have a halogen atom, which are represented by R^{Y2}, may have an ethereal oxygen atom,
the substituent Y represents a methyl group, an ethyl group, a linear propyl group, an aliphatic hydrocarbon group having a branched structure, an aliphatic hydrocarbon group having a cyclic structure, an aromatic ring group, an aliphatic heterocyclic group, a halogen atom, or —Si(R)₃, the methyl group, the ethyl group, the linear propyl group, the aliphatic hydrocarbon group having a branched structure, and the aliphatic hydrocarbon group having a cyclic structure, which are represented by the substituent Y, may have an ethereal oxygen atom,
R's each independently represent a methyl group, an ethyl group, or an aromatic ring group,
X¹ to X³ each independently represent a sulfur atom, an oxygen atom, a selenium atom, or a tellurium atom,
R¹ and R² each independently represent a hydrogen atom or a substituent, and
A¹ and A² each independently represent a group represented by Formula (A), 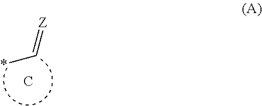
in Formula (A), * represents a bonding position, Z represents an oxygen atom, a sulfur atom, ═NR^{Z1}, or —CR^{Z2}R^{Z3}, R^{Z1} represents a hydrogen atom or a substituent, R^{Z2} and R^{Z3} each independently represent a cyano group, —SO₂R^{Z4}, —COOR^{Z5}, or —COR^{Z6}, R^{Z4} to R^{Z6} each independently represent an aliphatic hydrocarbon group which may have a substituent, an aromatic ring group which may have a substituent, or an aliphatic heterocyclic group which may have a substituent, and C represents a ring which contains 2 or more carbon atoms and may have a substituent.

2. The photoelectric conversion element according to claim 1,
wherein R^{Y1} represents a hydrogen atom, a methyl group, an ethyl group, a linear propyl group, an aliphatic hydrocarbon group having a branched structure, an aliphatic hydrocarbon group having a cyclic structure, an aromatic ring group, an aliphatic heterocyclic group, a halogen atom, or —Si(R)₃, and
the methyl group, the ethyl group, the linear propyl group, the aliphatic hydrocarbon group having a branched structure, and the aliphatic hydrocarbon group having a cyclic structure, which are represented by R^{Y1}, may have an ethereal oxygen atom,

3. The photoelectric conversion element according to claim 1,
wherein R^{Y2} represents a methyl group which may have a halogen atom, an ethyl group which may have a halogen atom, a linear propyl group which may have a halogen atom, an aliphatic hydrocarbon group having 3 to 6 carbon atoms, which has a branched structure and may have a halogen atom, an aliphatic hydrocarbon group having 3 to 10 carbon atoms which have a cyclic structure and may have a halogen atom, an acyl group which may have a halogen atom, —C≡C—Si(R)₃, an aromatic ring group having 10 or less carbon atoms which may have the substituent Y, an aliphatic heterocyclic group having 10 or less carbon atoms which may have the substituent Y, or —Si(R)₃, and
the methyl group which may have a halogen atom, the ethyl group which may have a halogen atom, the linear propyl group which may have a halogen atom, the aliphatic hydrocarbon group having 3 to 6 carbon atoms which has a branched structure and may have a halogen atom, and the aliphatic hydrocarbon group having 3 to 10 carbon atoms which has a cyclic structure and may have a halogen atom, which are represented by R^{Y2}, may have an ethereal oxygen atom.

4. The photoelectric conversion element according to claim 3,
wherein R^{Y2} represents a methyl group, an ethyl group, a propyl group, an isopropyl group, a tert-butyl group, a cyclopropyl group, a cyclobutyl group, a cyclopentyl group, a cyclohexyl group, an acetyl group, a trimethylsilylacetylene group, an aromatic hydrocarbon group having 10 or less carbon atoms which may have the substituent Y, or a trimethylsilyl group, and
the methyl group, the ethyl group, the propyl group, the isopropyl group, the tert-butyl group, which are represented by R^{Y2}, may have an ethereal oxygen atom.

5. The photoelectric conversion element according to claim 4,
wherein R^{Y2} represents a methyl group, an ethyl group, a propyl group, an isopropyl group, a tert-butyl group, a cyclopropyl group, a cyclobutyl group, a cyclopentyl group, a cyclohexyl group, an acetyl group, a trimethylsilylacetylene group, a phenyl group having 10 or less carbon atoms which may have the substituent Y, or a trimethylsilyl group, and the methyl group, the ethyl group, the propyl group, the isopropyl group, the tert-butyl group, which are represented by R^{Y2}, may have an ethereal oxygen atom.

6. The photoelectric conversion element according to claim 1,
wherein X¹ to X³ represent a sulfur atom.

7. The photoelectric conversion element according to claim 1,
wherein the compound represented by Formula (1) includes a compound represented by any of Formula (2) to Formula (8), 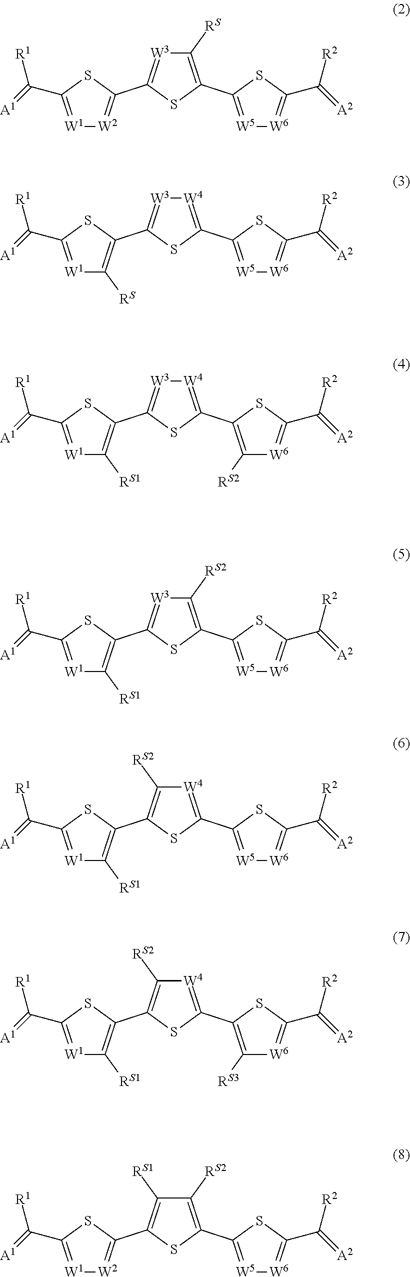
in Formula (2), W¹ to W³, W⁵, and W⁶ each independently represent —CR^{Y3}═ or a nitrogen atom, R^{Y3} represents a hydrogen atom, a fluorine atom, or a chlorine atom, R^{S} represents a methyl group which may have a halogen atom, an ethyl group which may have a halogen atom, a linear propyl group which may have a halogen atom, an aliphatic hydrocarbon group having 3 to 6 carbon atoms, which has a branched structure and may have a halogen atom, an aliphatic hydrocarbon group which has a cyclic structure and may have a halogen atom, an acyl group which may have a halogen atom, —C≡C—Si(R)₃, an aromatic ring group which may have the substituent Y, an aliphatic heterocyclic group which may have the substituent Y, or —Si(R)₃,
the methyl group which may have a halogen atom, the ethyl group which may have a halogen atom, the linear propyl group which may have a halogen atom, the aliphatic hydrocarbon group having 3 to 6 carbon atoms, which has a branched structure and may have a halogen atom, and the aliphatic hydrocarbon group which has a cyclic structure and may have a halogen atom, which are represented by R^{S}, may have an ethereal oxygen atom,
R¹ and R² each independently represent a hydrogen atom or a substituent, and
A¹ and A² each independently represent the group represented by Formula (A),
in Formula (3), W¹ and W³ to W⁶ each independently represent —CR^{Y3}═ or a nitrogen atom, R^{Y3} represents a hydrogen atom, a fluorine atom, or a chlorine atom, R^{S} represents a methyl group which may have a halogen atom, an ethyl group which may have a halogen atom, a linear propyl group which may have a halogen atom, an aliphatic hydrocarbon group having 3 to 6 carbon atoms, which has a branched structure and may have a halogen atom, an aliphatic hydrocarbon group which has a cyclic structure and may have a halogen atom, an acyl group which may have a halogen atom, —C≡C—Si(R)₃, an aromatic ring group which may have the substituent Y, an aliphatic heterocyclic group which may have the substituent Y, or —Si(R)₃,
the methyl group which may have a halogen atom, the ethyl group which may have a halogen atom, the linear propyl group which may have a halogen atom, the aliphatic hydrocarbon group having 3 to 6 carbon atoms, which has a branched structure and may have a halogen atom, and the aliphatic hydrocarbon group which has a cyclic structure and may have a halogen atom, which are represented by R^{S}, may have an ethereal oxygen atom,
R¹ and R² each independently represent a hydrogen atom or a substituent, and
A¹ and A² each independently represent the group represented by Formula (A),
in Formula (4), W¹, W³, W⁴, and W⁶ each independently represent —CR^{Y3}═ or a nitrogen atom, R^{Y3} represents a hydrogen atom, a fluorine atom, or a chlorine atom, R^{S1} and R^{S2} each independently represent a methyl group which may have a halogen atom, an ethyl group which may have a halogen atom, a linear propyl group which may have a halogen atom, an aliphatic hydrocarbon group having 3 to 6 carbon atoms, which has a branched structure and may have a halogen atom, an aliphatic hydrocarbon group which has a cyclic structure and may have a halogen atom, an acyl group which may have a halogen atom, —C≡C—Si(R)₃, an aromatic ring group which may have the substituent Y, an aliphatic heterocyclic group which may have the substituent Y, or —Si(R)₃,
the methyl group which may have a halogen atom, the ethyl group which may have a halogen atom, the linear propyl group which may have a halogen atom, the aliphatic hydrocarbon group having 3 to 6 carbon atoms, which has a branched structure and may have a halogen atom, and the aliphatic hydrocarbon group which has a cyclic structure and may have a halogen atom, which are represented by any of R^{S1} or R^{S2}, may have an ethereal oxygen atom,
R¹ and R² each independently represent a hydrogen atom or a substituent, and
A¹ and A² each independently represent the group represented by Formula (A),
in Formula (5), W¹, W³, W⁵, and W⁶ each independently represent —CR^{Y3}═ or a nitrogen atom, R^{Y3} represents a hydrogen atom, a fluorine atom, or a chlorine atom, R^{S1} and R^{S2} each independently represent a methyl group which may have a halogen atom, an ethyl group which may have a halogen atom, a linear propyl group which may have a halogen atom, an aliphatic hydrocarbon group having 3 to 6 carbon atoms, which has a branched structure and may have a halogen atom, an aliphatic hydrocarbon group which has a cyclic structure and may have a halogen atom, an acyl group which may have a halogen atom, —C≡C—Si(R)₃, an aromatic ring group which may have the substituent Y, an aliphatic heterocyclic group which may have the substituent Y, or —Si(R)₃,
the methyl group which may have a halogen atom, the ethyl group which may have a halogen atom, the linear propyl group which may have a halogen atom, the aliphatic hydrocarbon group having 3 to 6 carbon atoms, which has a branched structure and may have a halogen atom, and the aliphatic hydrocarbon group which has a cyclic structure and may have a halogen atom, which are represented by any of R^{S1} or R^{S2}, may have an ethereal oxygen atom,
R¹ and R² each independently represent a hydrogen atom or a substituent, and
A¹ and A² each independently represent the group represented by Formula (A),
in Formula (6), W¹ and W⁴ to W⁶ each independently represent —CR^{Y3}═ or a nitrogen atom, R^{Y3} represents a hydrogen atom, a fluorine atom, or a chlorine atom, R^{S1} and R^{S2} each independently represent a methyl group which may have a halogen atom, an ethyl group which may have a halogen atom, a linear propyl group which may have a halogen atom, an aliphatic hydrocarbon group having 3 to 6 carbon atoms, which has a branched structure and may have a halogen atom, an aliphatic hydrocarbon group which has a cyclic structure and may have a halogen atom, an acyl group which may have a halogen atom, —C≡C—Si(R)₃, an aromatic ring group which may have the substituent Y, an aliphatic heterocyclic group which may have the substituent Y, or —Si(R)₃,
the methyl group which may have a halogen atom, the ethyl group which may have a halogen atom, the linear propyl group which may have a halogen atom, the aliphatic hydrocarbon group having 3 to 6 carbon atoms, which has a branched structure and may have a halogen atom, and the aliphatic hydrocarbon group which has a cyclic structure and may have a halogen atom, which are represented by any of R^{S1} or R^{S2}, may have an ethereal oxygen atom,
R¹ and R² each independently represent a hydrogen atom or a substituent, and
A¹ and A² each independently represent the group represented by Formula (A),
in Formula (7), W¹, W⁴, and W⁶ each independently represent —CR^{Y3}═ or a nitrogen atom, R^{Y3} represents a hydrogen atom, a fluorine atom, or a chlorine atom, R^{S1} to R^{S3} each independently represent a methyl group which may have a halogen atom, an ethyl group which may have a halogen atom, a linear propyl group which may have a halogen atom, an aliphatic hydrocarbon group having 3 to 6 carbon atoms, which has a branched structure and may have a halogen atom, an aliphatic hydrocarbon group which has a cyclic structure and may have a halogen atom, an acyl group which may have a halogen atom, —C≡C—Si(R)₃, an aromatic ring group which may have the substituent Y, an aliphatic heterocyclic group which may have the substituent Y, or —Si(R)₃,
the methyl group which may have a halogen atom, the ethyl group which may have a halogen atom, the linear propyl group which may have a halogen atom, the aliphatic hydrocarbon group having 3 to 6 carbon atoms, which has a branched structure and may have a halogen atom, and the aliphatic hydrocarbon group which has a cyclic structure and may have a halogen atom, which are represented by any of R^{S1} to R^{S3}, may have an ethereal oxygen atom,
R¹ and R² each independently represent a hydrogen atom or a substituent, and
A¹ and A² each independently represent the group represented by Formula (A),
in Formula (8), W¹, W², W⁵, and W⁶ each independently represent —CR^{Y3}═ or a nitrogen atom, R^{Y3} represents a hydrogen atom, a fluorine atom, or a chlorine atom, R^{S1} and R^{S2} each independently represent a methyl group which may have a halogen atom, an ethyl group which may have a halogen atom, a linear propyl group which may have a halogen atom, an aliphatic hydrocarbon group having 3 to 6 carbon atoms, which has a branched structure and may have a halogen atom, an aliphatic hydrocarbon group which has a cyclic structure and may have a halogen atom, an acyl group which may have a halogen atom, —C≡C—Si(R)₃, an aromatic ring group which may have the substituent Y, an aliphatic heterocyclic group which may have the substituent Y, or —Si(R)₃,
the methyl group which may have a halogen atom, the ethyl group which may have a halogen atom, the linear propyl group which may have a halogen atom, the aliphatic hydrocarbon group having 3 to 6 carbon atoms, which has a branched structure and may have a halogen atom, and the aliphatic hydrocarbon group which has a cyclic structure and may have a halogen atom, which are represented by any of R^{S1} or R^{S2}, may have an ethereal oxygen atom,
R¹ and R² each independently represent a hydrogen atom or a substituent, and
A¹ and A² each independently represent the group represented by Formula (A).

8. The photoelectric conversion element according to claim 1,
wherein A¹ and A² each independently represent a group represented by Formula (A1) or a group represented bv Formula (A2), 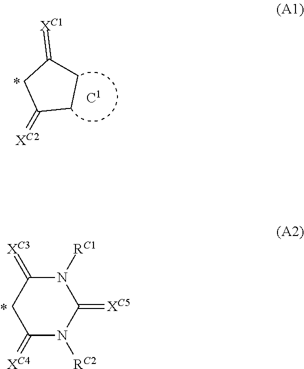
in Formula (A1), * represents a bonding position, X^{C1} and X^{C2} each independently represent an oxygen atom, a sulfur atom, —NR^{Z1}, or ═CR^{Z2}R^{Z3}, R^{Z1} represents a hydrogen atom or a substituent, R^{Z2} and R^{Z3} each independently represent a cyano group, —SO₂R^{Z4}, —COOR^{Z5}, or —COR^{Z6}, R^{Z4} to R^{Z6} each independently represent an aliphatic hydrocarbon group which may have a substituent, an aromatic ring group which may have a substituent, or an aliphatic heterocyclic group which may have a substituent, and C¹ represents an aromatic ring group which contains 2 or more carbon atoms and may have a substituent, or an aliphatic heterocyclic group which contains 2 or more carbon atoms and may have a substituent,
in Formula (A2), * represents a bonding position, X^{C3} to X^{C5} each independently represent an oxygen atom, a sulfur atom, ═NR^{Z1}, or —CR^{Z2}R^{Z3}, R^{Z1} represents a hydrogen atom or a substituent, R^{Z2} and R^{Z3} each independently represent a cyano group, —SO₂R²⁴, —COOR^{Z5}, or —COR^{Z6}, R^{Z4} to R^{Z6} each independently represent an aliphatic hydrocarbon group which may have a substituent, an aromatic ring group which may have a substituent, or an aliphatic heterocyclic group which may have a substituent, and R^{C1} and R^{C2} each independently represent a hydrogen atom or a substituent.

9. The photoelectric conversion element according to claim 1,
wherein the photoelectric conversion film further contains an n-type organic semiconductor, and
the photoelectric conversion film has a bulk hetero structure formed in a state where the compound represented by Formula (1) and the n-type organic semiconductor are mixed.

10. The photoelectric conversion element according to claim 9,
wherein the n-type organic semiconductor includes fullerenes selected from the group consisting of a fullerene and a derivative of the fullerene.

11. The photoelectric conversion element according to claim 1,
wherein the photoelectric conversion film further contains a p-type organic semiconductor.

12. The photoelectric conversion element according to claim 1,
wherein the photoelectric conversion film further contains a coloring agent.

13. The photoelectric conversion element according to claim 1, further comprising:
one or more interlayers between the conductive film and the transparent conductive film, in addition to the photoelectric conversion film.

14. An imaging element comprising:
the photoelectric conversion element according to claim 1.

15. An optical sensor comprising:
the photoelectric conversion element according to claim 1.

16. A compound represented by Formula (1), 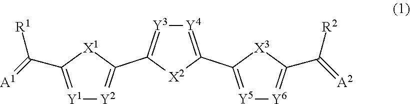
in Formula (1), Y¹to Y⁶ each independently represent —CR^{Y1}═ or —N═, R^{Y1} represents a hydrogen atom or a substituent, at least one of Y¹, Y², Y³, Y⁴, Y⁵, or Y⁶ represents —CR^{Y2}═, R^{Y2} represents a methyl group which may have a halogen atom, an ethyl group which may have a halogen atom, a linear propyl group which may have a halogen atom, an aliphatic hydrocarbon group having 3 to 6 carbon atoms, which has a branched structure and may have a halogen atom, an aliphatic hydrocarbon group which has a cyclic structure and may have a halogen atom, an acyl group which may have a halogen atom, —C≡C—Si(R)₃, an aromatic ring group which may have a substituent Y, an aliphatic heterocyclic group which may have a substituent Y, or —Si(R)₃,
the methyl group which may have a halogen atom, the ethyl group which may have a halogen atom, the linear propyl group which may have a halogen atom, the aliphatic hydrocarbon group having 3 to 6 carbon atoms, which has a branched structure and may have a halogen atom, and the aliphatic hydrocarbon group which has a cyclic structure and may have a halogen atom, which are represented by R^{Y2}, may have an ethereal oxygen atom,
the substituent Y represents a methyl group, an ethyl group, a linear propyl group, an aliphatic hydrocarbon group having a branched structure, an aliphatic hydrocarbon group having a cyclic structure, an aromatic ring group, an aliphatic heterocyclic group, a halogen atom, or —Si(R)₃, the methyl group, the ethyl group, the linear propyl group, the aliphatic hydrocarbon group having a branched structure, and the aliphatic hydrocarbon group having a cyclic structure, which are represented by the substituent Y, may have an ethereal oxygen atom,
R's each independently represent a methyl group, an ethyl group, or an aromatic ring group,
X¹ to X³ each independently represent a sulfur atom, an oxygen atom, a selenium atom, or a tellurium atom,
R¹ and R² each independently represent a hydrogen atom or a substituent, and
A¹ and A² each independently represent a group represented by Formula (A), 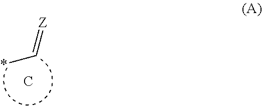
in Formula (A), * represents a bonding position, Z represents an oxygen atom, a sulfur atom, ═NR^{Z1}, or ═CR^{Z2}R^{Z3}, R^{Z1} represents a hydrogen atom or a substituent, R^{Z2} and R^{Z3} each independently represent a cyano group, —SO₂R^{Z4}, —COOR^{Z5}, or —COR^{Z6}, R^{Z4} to R^{Z6} each independently represent an aliphatic hydrocarbon group which may have a substituent, an aromatic ring group which may have a substituent, or an aliphatic heterocyclic group which may have a substituent, and C represents a ring which contains 2 or more carbon atoms and may have a substituent.

17. The compound according to claim 16,
wherein R^{Y1} represents a hydrogen atom, a methyl group, an ethyl group, a linear propyl group, an aliphatic hydrocarbon group having a branched structure, an aliphatic hydrocarbon group having a cyclic structure, an aromatic ring group, an aliphatic heterocyclic group, a halogen atom, or —Si(R)₃, and
the methyl group, the ethyl group, the linear propyl group, the aliphatic hydrocarbon group having a branched structure, and the aliphatic hydrocarbon group having a cyclic structure, which are represented by R^{Y1}, may have an ethereal oxygen atom.

18. The compound according to claim 16,
wherein R^{Y2} represents a methyl group which may have a halogen atom, an ethyl group which may have a halogen atom, a linear propyl group which may have a halogen atom, an aliphatic hydrocarbon group having 3 to 6 carbon atoms which has a branched structure and may have a halogen atom, an aliphatic hydrocarbon group having 3 to 10 carbon atoms which have a cyclic structure and may have a halogen atom, an acyl group which may have a halogen atom, —C≡C—Si(R)₃, an aromatic ring group having 10 or less carbon atoms which may have the substituent Y, an aliphatic heterocyclic group having 10 or less carbon atoms which may have the substituent Y, or —Si(R)₃, and
the methyl group which may have a halogen atom, the ethyl group which may have a halogen atom, the linear propyl group which may have a halogen atom, the aliphatic hydrocarbon group having 3 to 6 carbon atoms which has a branched structure and may have a halogen atom, and the aliphatic hydrocarbon group having 3 to 10 carbon atoms which has a cyclic structure and may have a halogen atom, which are represented by R^{Y2}, may have an ethereal oxygen atom.

19. The compound according to claim 18,
wherein R^{Y2} represents a methyl group, an ethyl group, a propyl group, an isopropyl group, a tert-butyl group, a cyclopropyl group, a cyclobutyl group, a cyclopentyl group, a cyclohexyl group, an acetyl group, a trimethylsilylacetylene group, an aromatic hydrocarbon group having 10 or less carbon atoms which may have the substituent Y, or a trimethylsilyl group, and
the methyl group, the ethyl group, the propyl group, the isopropyl group, the tert-butyl group, which are represented by R^{Y2}, may have an ethereal oxygen atom.

20. The compound according to claim 19,
wherein R^{Y2} represents a methyl group, an ethyl group, a propyl group, an isopropyl group, a tert-butyl group, a cyclopropyl group, a cyclobutyl group, a cyclopentyl group, a cyclohexyl group, an acetyl group, a trimethylsilylacetylene group, a phenyl group having 10 or less carbon atoms which may have the substituent Y, or a trimethylsilyl group, and
the methyl group, the ethyl group, the propyl group, the isopropyl group, the tert-butyl group, which are represented by R^{Y2}, may have an ethereal oxygen atom.

21. The compound according to claim 16,
wherein X¹ to X³ represent a sulfur atom.

22. The compound according to claim 16,
wherein the compound represented by Formula (1) includes a compound represented by any of Formula (2) to Formula (8), 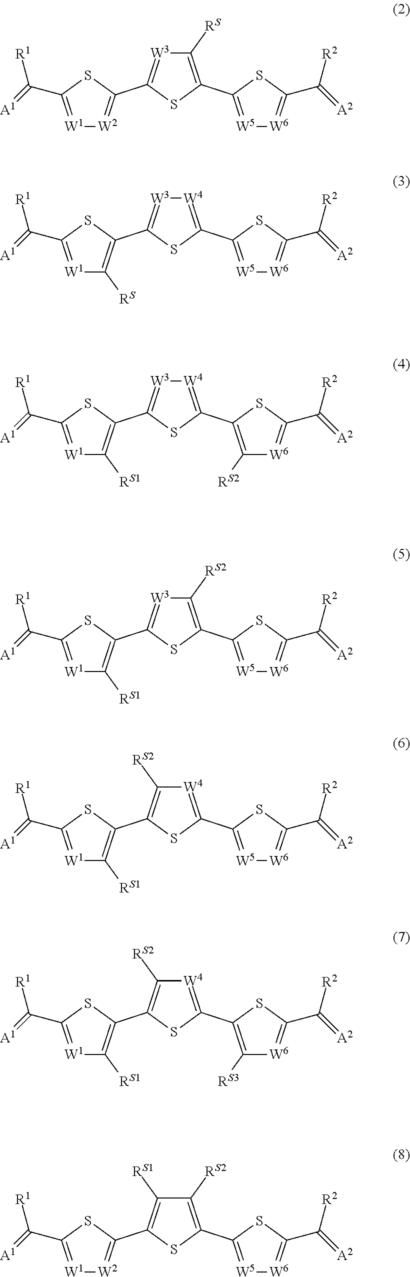
in Formula (2), W¹ to W³, W⁵, and W⁶ each independently represent —CR^{Y3}═ or a nitrogen atom, R^{Y3} represents a hydrogen atom, a fluorine atom, or a chlorine atom, R^{S} represents a methyl group which may have a halogen atom, an ethyl group which may have a halogen atom, a linear propyl group which may have a halogen atom, an aliphatic hydrocarbon group having 3 to 6 carbon atoms, which has a branched structure and may have a halogen atom, an aliphatic hydrocarbon group which has a cyclic structure and may have a halogen atom, an acyl group which may have a halogen atom, —C≡C—Si(R)₃, an aromatic ring group which may have the substituent Y, an aliphatic heterocyclic group which may have the substituent Y, or —Si(R)₃,
the methyl group which may have a halogen atom, the ethyl group which may have a halogen atom, the linear propyl group which may have a halogen atom, the aliphatic hydrocarbon group having 3 to 6 carbon atoms, which has a branched structure and may have a halogen atom, and the aliphatic hydrocarbon group which has a cyclic structure and may have a halogen atom, which are represented by R^{S}, may have an ethereal oxygen atom,
R¹ and R² each independently represent a hydrogen atom or a substituent, and
A¹ and A² each independently represent the group represented by Formula (A),
in Formula (3), W¹ and W³ to W⁶ each independently represent —CR^{Y3}═ or a nitrogen atom, R^{Y3} represents a hydrogen atom, a fluorine atom, or a chlorine atom, R^{S} represents a methyl group which may have a halogen atom, an ethyl group which may have a halogen atom, a linear propyl group which may have a halogen atom, an aliphatic hydrocarbon group having 3 to 6 carbon atoms, which has a branched structure and may have a halogen atom, an aliphatic hydrocarbon group which has a cyclic structure and may have a halogen atom, an acyl group which may have a halogen atom, —C≡C—Si(R)₃, an aromatic ring group which may have the substituent Y, an aliphatic heterocyclic group which may have the substituent Y, or —Si(R)₃,
the methyl group which may have a halogen atom, the ethyl group which may have a halogen atom, the linear propyl group which may have a halogen atom, the aliphatic hydrocarbon group having 3 to 6 carbon atoms, which has a branched structure and may have a halogen atom, and the aliphatic hydrocarbon group which has a cyclic structure and may have a halogen atom, which are represented by R^{S}, may have an ethereal oxygen atom,
R¹ and R² each independently represent a hydrogen atom or a substituent, and
A¹ and A² each independently represent the group represented by Formula (A),
in Formula (4), W¹, W³, W⁵, and W⁶ each independently represent —CR^{Y3}═ or a nitrogen atom, R^{Y3} represents a hydrogen atom, a fluorine atom, or a chlorine atom, R^{S1} and R^{S2} each independently represent a methyl group which may have a halogen atom, an ethyl group which may have a halogen atom, a linear propyl group which may have a halogen atom, an aliphatic hydrocarbon group having 3 to 6 carbon atoms, which has a branched structure and may have a halogen atom, an aliphatic hydrocarbon group which has a cyclic structure and may have a halogen atom, an acyl group which may have a halogen atom, —C≡C—Si(R)₃, an aromatic ring group which may have the substituent Y, an aliphatic heterocyclic group which may have the substituent Y, or —Si(R)₃,
the methyl group which may have a halogen atom, the ethyl group which may have a halogen atom, the linear propyl group which may have a halogen atom, the aliphatic hydrocarbon group having 3 to 6 carbon atoms, which has a branched structure and may have a halogen atom, and the aliphatic hydrocarbon group which has a cyclic structure and may have a halogen atom, which are represented by any of R^{S1} or R^{S2}, may have an ethereal oxygen atom,
R¹ and R² each independently represent a hydrogen atom or a substituent, and
A¹ and A² each independently represent the group represented by Formula (A),
in Formula (5), W¹, W³, W⁵, and W⁶ each independently represent —CR^{Y3}═ or a nitrogen atom, R^{Y3} represents a hydrogen atom, a fluorine atom, or a chlorine atom, R^{S1} and R^{S2} each independently represent a methyl group which may have a halogen atom, an ethyl group which may have a halogen atom, a linear propyl group which may have a halogen atom, an aliphatic hydrocarbon group having 3 to 6 carbon atoms, which has a branched structure and may have a halogen atom, an aliphatic hydrocarbon group which has a cyclic structure and may have a halogen atom, an acyl group which may have a halogen atom, —C≡C—Si(R)₃, an aromatic ring group which may have the substituent Y, an aliphatic heterocyclic group which may have the substituent Y, or —Si(R)₃,
the methyl group which may have a halogen atom, the ethyl group which may have a halogen atom, the linear propyl group which may have a halogen atom, the aliphatic hydrocarbon group having 3 to 6 carbon atoms, which has a branched structure and may have a halogen atom, and the aliphatic hydrocarbon group which has a cyclic structure and may have a halogen atom, which are represented by any of R^{S1} or R^{S2}, may have an ethereal oxygen atom,
R¹ and R² each independently represent a hydrogen atom or a substituent, and
A¹ and A² each independently represent the group represented by Formula (A),
in Formula (6), W¹ and W⁵ to W⁶ each independently represent —CR^{Y3}═ or a nitrogen atom, R^{Y3} represents a hydrogen atom, a fluorine atom, or a chlorine atom, R^{S1} and R^{S2} each independently represent a methyl group which may have a halogen atom, an ethyl group which may have a halogen atom, a linear propyl group which may have a halogen atom, an aliphatic hydrocarbon group having 3 to 6 carbon atoms, which has a branched structure and may have a halogen atom, an aliphatic hydrocarbon group which has a cyclic structure and may have a halogen atom, an acyl group which may have a halogen atom, —C≡C—Si(R)₃, an aromatic ring group which may have the substituent Y, an aliphatic heterocyclic group which may have the substituent Y, or —Si(R)₃,
the methyl group which may have a halogen atom, the ethyl group which may have a halogen atom, the linear propyl group which may have a halogen atom, the aliphatic hydrocarbon group having 3 to 6 carbon atoms, which has a branched structure and may have a halogen atom, and the aliphatic hydrocarbon group which has a cyclic structure and may have a halogen atom, which are represented by any of R^{S1} or R^{S2}, may have an ethereal oxygen atom,
R¹ and R² each independently represent a hydrogen atom or a substituent, and
A¹ and A² each independently represent the group represented by Formula (A),
in Formula (7), W¹, W⁵, and W⁶ each independently represent —CR^{Y3}═ or a nitrogen atom, R^{Y3} represents a hydrogen atom, a fluorine atom, or a chlorine atom, R^{S1} to R^{S3} each independently represent a methyl group which may have a halogen atom, an ethyl group which may have a halogen atom, a linear propyl group which may have a halogen atom, an aliphatic hydrocarbon group having 3 to 6 carbon atoms, which has a branched structure and may have a halogen atom, an aliphatic hydrocarbon group which has a cyclic structure and may have a halogen atom, an acyl group which may have a halogen atom, —C≡C—Si(R)₃, an aromatic ring group which may have the substituent Y, an aliphatic heterocyclic group which may have the substituent Y, or —Si(R)₃,
the methyl group which may have a halogen atom, the ethyl group which may have a halogen atom, the linear propyl group which may have a halogen atom, the aliphatic hydrocarbon group having 3 to 6 carbon atoms, which has a branched structure and may have a halogen atom, and the aliphatic hydrocarbon group which has a cyclic structure and may have a halogen atom, which are represented by any of R^{S1} to R^{S3}, may have an ethereal oxygen atom,
R¹ and R² each independently represent a hydrogen atom or a substituent, and
A¹ and A² each independently represent the group represented by Formula (A),
in Formula (8), W¹, W², W³, and W⁶ each independently represent —CR^{Y3}═ or a nitrogen atom, R^{Y3} represents a hydrogen atom, a fluorine atom, or a chlorine atom, R^{S1} and R^{S2} each independently represent a methyl group which may have a halogen atom, an ethyl group which may have a halogen atom, a linear propyl group which may have a halogen atom, an aliphatic hydrocarbon group having 3 to 6 carbon atoms, which has a branched structure and may have a halogen atom, an aliphatic hydrocarbon group which has a cyclic structure and may have a halogen atom, an acyl group which may have a halogen atom, —C≡C—Si(R)₃, an aromatic ring group which may have the substituent Y, an aliphatic heterocyclic group which may have the substituent Y, or —Si(R)₃,
the methyl group which may have a halogen atom, the ethyl group which may have a halogen atom, the linear propyl group which may have a halogen atom, the aliphatic hydrocarbon group having 3 to 6 carbon atoms, which has a branched structure and may have a halogen atom, and the aliphatic hydrocarbon group which has a cyclic structure and may have a halogen atom, which are represented by any of R^{S1} or R^{S2}, may have an ethereal oxygen atom,
R¹ and R² each independently represent a hydrogen atom or a substituent, and
A¹ and A² each independently represent the group represented by Formula (A).

23. The compound according to claim 16,
wherein A¹ and A² each independently represent a group represented by Formula (A1) or a group represented by Formula (A2), 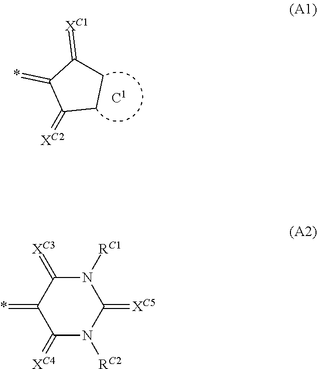
in Formula (A1), * represents a bonding position, X^{C1} and X^{C2} each independently represent an oxygen atom, a sulfur atom, ═NR^{Z1}, or ═CR^{Z2}R^{Z3}, R^{Z1} represents a hydrogen atom or a substituent, R^{Z2} and R^{Z3} each independently represent a cyano group, —SO₂R_{Z4}, —COOR^{Z5}, or —COR^{Z6}, R^{Z4} to R^{Z6} each independently represent an aliphatic hydrocarbon group which may have a substituent, an aromatic ring group which may have a substituent, or an aliphatic heterocyclic group which may have a substituent, and C¹ represents an aromatic ring group which contains 2 or more carbon atoms and may have a substituent, or an aliphatic heterocyclic group which contains 2 or more carbon atoms and may have a substituent,
in Formula (A2), * represents a bonding position, X^{C3} to X^{C5} each independently represent an oxygen atom, a sulfur atom, ═NR^{Z1}, or —CR^{Z2}R^{Z3}, R^{Z1} represents a hydrogen atom or a substituent, R^{Z2} and R^{Z3} each independently represent a cyano group, —SO₂R^{Z4}, —COOR^{Z5}, or —COR^{Z6}, R^{Z4} to R^{Z6} each independently represent an aliphatic hydrocarbon group which may have a substituent, an aromatic ring group which may have a substituent, or an aliphatic heterocyclic group which may have a substituent, and R^{C1} and R^{C2} each independently represent a hydrogen atom or a substituent.
